(19) United States
(12) Patent Application Publication
(10) Pub. No.: US 2026/0151455 A1
(43) Pub. Date: Jun. 4, 2026

(54) ALTERING INFLAMMATORY STATES OF IMMUNE CELLS IN VIVO BY MODULATING CELLULAR ACTIVATION STATES

(71) Applicant: Fred Hutchinson Cancer Center, Seattle, WA (US)

(72) Inventor: Matthias Stephan, Seattle, WA (US)

(73) Assignee: Fred Hutchinson Cancer Center, Seattle, WA (US)

(21) Appl. No.: 19/535,440

(22) Filed: Feb. 10, 2026

Related U.S. Application Data
(62) Division of application No. 16/963,119, now Pat. No. 12,576,131.
(60) Provisional application No. 62/618,908, filed on Jan. 18, 2018.

(51) Int. Cl. A61K 38/17; A61K 9/14; A61K 9/51; A61K 31/7088; A61K 38/45; A61K 45/06; C12N 15/11

(52) U.S. Cl. CPC A61K 38/1709; A61K 9/146; A61K 9/5146; A61K 31/7088; A61K 38/45; A61K 45/06; C12N 15/11; C12Y 207/1101

(57) ABSTRACT

Systems and methods to modulate the activation state of immune cells in vivo are described. The systems and methods can be used to transform immunosuppressive macrophages that support cancer growth and metastasis into highly activated tumoricidal macrophages.

### CROSS-REFERENCE TO RELATED APPLICATIONS

This application is a Divisional Application of U.S. patent application Ser. No. 16/963,119, filed Jul. 17, 2020, which is a U.S. National Phase Application based on International Patent Application No. PCT/US2019/014209, filed Jan. 18, 2019, which claims the benefit of priority to U.S. Provisional Patent Application No. 62/618,908, filed Jan. 18, 2018, each of which is incorporated by reference herein in its entirety.

### STATEMENT REGARDING SEQUENCE LISTING

The Sequence Listing associated with this application is provided in xml format in lieu of a paper copy and is hereby incorporated by reference into the specification. The name of the xml file containing the Sequence Listing is F053-0076USD1Sequence_Listing.xml. The xml file is 159,744 bytes, was created on or about Feb. 10, 2026 and is being submitted electronically via Patent Center.

### FIELD OF THE DISCLOSURE

The current disclosure provides systems and methods to modulate the activation state of immune cells in vivo. The systems and methods can be used to transform immunosuppressive macrophages that support cancer growth and metastasis into highly activated tumoricidal macrophages.

### BACKGROUND OF THE DISCLOSURE

A number of adverse physiological conditions are associated with immune system activation (e.g., autoimmune disorders) or immune system suppression (e.g., cancer). For example, macrophages are key immune effector cells that infiltrate cancerous tissue in high numbers. Within the tumor microenvironment, however, macrophages undergo a switch from an activated tumoricidal state to an immunosuppressive phenotype that actually facilitates tumor growth and metastasis. Pollard, Nat Rev Cancer 4, 71-78 (2004); Mantovani, et al., Nat Rev Clin Oncol (2017).

Understanding that immunosuppressed macrophages within the tumor microenvironment facilitate cancer growth and metastasis, much effort has been devoted to developing therapies that target immunosuppressive tumor-associated macrophages (TAMs). Many efforts to address TAMs have focused on killing the TAMs to alleviate immunosuppression in the tumor microenvironment. With this approach, however, the TAMs are simply replaced with newly-arriving macrophages at the tumor environment. Moreover, even when successful at killing some TAMs, most therapeutics developed to date have not been able to sufficiently penetrate into the tumor microenvironment. While some small molecule drugs and antibodies have shown some success, these approaches have suppressed all macrophages in the body, inducing dangerous side effects. Bowman & Joyce, Immunotherapy 6, 663-666 (2014). Thus, as is understood by everyone affected by cancer, more effective treatment strategies with fewer side effects are greatly needed.

### SUMMARY OF THE DISCLOSURE

The current disclosure provides systems and methods to modulate the function of immune cells in vivo. In particular embodiments, the systems and methods are used to reverse the immunosuppressive, tumor supporting state of tumor-associated macrophages (TAMs) and turn these TAMs into highly activated, tumor cell-killing macrophages. Thus, the systems and methods disclosed herein do not simply aim to kill TAMs, but instead redirects their activity from tumor-promoting to tumor-destroying. In particular embodiments, the systems and methods are used as a therapeutic to induce the killing of cancer cells and/or to reduce or prevent the growth or development of new cancer cells. Data disclosed herein shows that these systems and methods are able to completely eradicate and suppress ovarian cancer, a notoriously difficult cancer type to control.

The systems and methods disclosed herein can be used to alter the immunosuppressive state in a tumor, providing a mechanism to restructure the tumor microenvironment. In these embodiments, a restructured tumor microenvironment can render a tumor more susceptible to a companion treatment, such as a vaccine, a chimeric antigen receptor (CAR) therapy, and/or chemotherapy.

Importantly, the systems and methods disclosed herein can be used locally at the tumor microenvironment obviating the need to resort to systemic treatments that globally disrupt immune system homeostasis. Moreover, particular embodiments have been optimized to successfully infiltrate the tumor microenvironment.

Particular embodiments alter the activation states of immune cells in vivo by utilizing a particle to deliver nucleotides encoding activation regulators, such as transcription factors. A particularly useful particle has a positive core and a neutral or negatively-charged surface and delivers nucleotides encoding the transcription factor interferon-regulatory factor 5 (IRF5) in combination with the kinase IKKβ. A particle size of <130 nm ensures tumor infiltration. Moreover, the particles can include a TAM targeting ligand to direct more selective uptake of the particles by TAMs. As one example, TAMs express CD206, a cellular surface receptor that can be targeted by including mannose on the surface of the particles.

### BRIEF DESCRIPTION OF THE FIGURES

Many of the figures submitted herein are better understood in color. Applicants consider the color versions of the drawings as part of the original submission and reserve the right to present color images of the drawings in later proceedings.

FIGS. 1A, 1B. Scheme to genetically transform tumor-associated macrophages (TAMs) into tumoricidal cells using targeted mRNA nanoparticles. (FIG. 1A) An injectable nanocarrier was developed to deliver in vitro transcribed mRNA encoding M1-polarizing transcription factors as a new method to rationally reprogram TAMs for therapeutic purposes without causing systemic toxicity. Illustrated is the first planned clinical application, designed to treat ovarian cancer patients with repeated intraperitoneal infusions of mRNA nanoparticles. (FIG. 1B) Scheme to genetically reprogram intracranial TAMs into tumoricidal macrophages using targeted mRNA nanoparticles.

FIGS. 2A-2K. Nanoparticles carrying mRNA encoding IRF5 and IKKβ can imprint a pro-inflammatory M1-like phenotype. (FIG. 2A) Design of macrophage-targeted polymeric NPs formulated with mRNAs encoding key regulators of macrophage polarization. The particles consist of a PbAE-mRNA polyplex core coated with a layer of PGA-Di-mannose, which targets the particles to mannose receptors (CD206) expressed by M2-like macrophages. Also depicted is the synthetic mRNA encapsulated in the NP, which is engineered to encode the reprogramming transcription factors. (FIG. 2B) Transmission electron microscopy of a population of NPs (scale bar 200 nm) and a single NP (inset, scale bar 50 nm). (FIG. 2C) Size distributions of NPs, measured using a NanoSight NS300 instrument. (FIG. 2D) NPs demonstrated high transfection (46%) of bone marrow-derived macrophages (BMDMs) after 1 h exposure. (FIG. 2E) Gene-transfer efficiencies into bone marrow derived macrophages (BMDM) measured by flow cytometry 24 hours after nanoparticle transfection. (FIG. 2F) Relative viability of NP transfected and untransfected macrophages (assessed by staining with Annexin V and PI). N.s.; non-significant. (FIG. 2G) Expression kinetics of codon-optimized IRF5 mRNA (blue, left Y axis) and endogenous IRF5 mRNA (black, right Y axis) measured by qRT-PCR, n=3 for each time point. (FIG. 2H) Timelines depicting NP transfection protocols and culture conditions for the BMDMs used in FIGS. 2I-2K. (FIG. 2I) Gene expression profiles of IRF5/IKKβ NP-transfected macrophages compared to signature M1 cells stimulated with the Toll-like Receptor 6 agonist MPLA. Results are depicted as a Volcano plot that shows the distribution of the fold changes in gene expression. M1 signature genes are indicated. P value of overlap between IRF5/IKKβ NP-transfected macrophages and the M1 signature gene set was determined by GSEA. (FIG. 2J) Heat map of M1 signature gene expression in macrophages cultured in IL-4 versus cells cultured in IL-4 and transfected with IRF5/IKKβ NPs. (FIG. 2K) Box plots showing mean counts for indicated genes and S.E.M.

FIG. 3. In vitro screening of the effect of different members of the interferon-regulatory factor (IRF) family (delivered in combination with or without their activating kinase) on the phenotype of mouse macrophages. BMDMs from C57BL/6 mice were incubated in M-CSF conditioning media and transfected with mRNA-PBAE NPs carrying synthetic mRNA encoding (1) control GFP, (2) murine IRF5, (3) murine IRF5 and the IKKβ kinase, which phosphorylates IRF5, (4) murine IRF8 and the IKKβ kinase, (5) murine IRF8 K310R, which is a mutant of IRF8, with a Lys-310 to Arg (K310R) conversion (White et al., J Biol Chem. 2016 Jun. 24), or (6) murine IRF7/3 (5D). This fusion protein includes the DNA binding domain (DBD) and constitutively active domain (CAD) of IRF-7 and the nuclear export signal (NES) and IRF association domain of IRF3 (Lin et al., Molecular and Cellular Biology. 18.5, 1998). Two days after NP transfection, cells were harvested for flow cytometric analysis for the TAM-associated macrophage marker Egr2 and the activated macrophage marker CD38. Based on this in vitro screen, NPs co-delivering mRNA encoding mIRF5 and IKKβ kinase were chosen for the remainder of in vitro and therapeutic in vivo experiments described herein.

FIGS. 4A-4J. Repeated intraperitoneal injections of mRNA nanocarriers delivering IRF5 and IKKβ genes into macrophages more than doubles mean survival of mice with disseminated ovarian cancer. (FIG. 4A) Time lines and dosing regimens. Arrows indicate time of I.P. injection. (FIG. 4B) Sequential bioluminescence imaging of tumor growth in control and treated mice. (FIG. 4C) Kaplan-Meier survival curves for treated versus control mice. Statistical analysis was performed using the log-rank test. (FIG. 4D) Flow cytometric quantitation of in vivo transfection rates in different immune cell subpopulations 48 hours after a single i.p. dose of D-mannose-coated NPs carrying GFP mRNA as a control: macrophages (CD45+, CD11b+, MHCII+, CD11c−, Ly6C−/low, Ly6G−), monocytes (CD45+, CD11b+, MHCII+, CD11c−, Ly6C+, Ly6G−), neutrophils (CD45+, CD11b+, MHCII+, CD11c−, Ly6G+), CD4+ T cells (CD45+, TCR-β chain+, CD4+, CD8−), CD8+ T cells (CD45+, TCR-β chain+, CD4−, CD8+), and natural killer cells (CD45+, TCR-β chain−, CD49b+) were measured. (FIG. 4E) Flow cytometric analysis of macrophage phenotypes in the peritoneum of mice with disseminated ID8 ovarian cancer. Animals were either treated with 4 doses of IRF5/IKKβ NPs or PBS. (FIG. 4F) Box plots summarizing relative percent (left panel) and absolute numbers (right panel) of Ly6C−, F4/80+, and CD206+ (M2-like) macrophages. (FIG. 4G) Corresponding numbers for Ly6C−, F4/80+, and CD206− (M1-like) macrophages. (FIG. 4H) Representative hematoxylin and eosin-stained sections of ovarian tumor-infiltrated mesenteries isolated from PBS controls (top panel) or IRF5/IKKβ NP-treated animals (bottom panel; scale bar 100 μm). 10-fold magnifications of representative malignant lesions are shown on the right (scale bar 50 μm). (FIG. 4I) Luminex assay measuring cytokines produced by isolated peritoneal macrophages from each treatment group. CD11b+, F4/80+ peritoneal macrophages were isolated by fluorescence activated cell sorting, and cultured ex vivo. After 24 hours, cell culture supernatants were collected. In parallel experiments, FACS-sorted CD11b+, F4/80+ peritoneal macrophages were directly analyzed by pRT-PCR to determine expression levels of four master regulators of the macrophage phenotypes (SerpinB2, Retnla, Ccl11, and Ccl5). Results are summarized as box plots in FIG. 4J.

FIGS. 5A-5F. Macrophage-programming mRNA nanocarriers are highly biocompatible and safe for repeated dosing. (FIG. 5A) In vivo biodistribution of macrophage-targeted IRF5/IKKβ NPs following i.p. administration. NP-delivered (codon optimized) mRNA was detected by qPCR 24 hours after a single injection of particles containing 50 μg mRNA. (FIG. 5B) Schematic representation of the experimental timeline. * Twenty-four hours after the last dose, mice were euthanized by CO₂ inhalation. Blood was collected through retro-orbital bleeding into heparin coated tubes for serum chemistry and complete blood count. Necrospy was performed for histological analysis of liver, spleen, pancreas, mesentery and omentum, stomach, and urinary bladder. (FIG. 5C) Representative hematoxylin and eosin-stained sections of various organs isolated from controls or NP-treated animals. Scale bar, 100 μm. Lesions found in the NP-treated animals are shown and described here based on analysis by a Comparative Pathologist. The relevant findings for each numbered image is: [1] Discrete foci of cellular infiltrates largely composed of mononuclear cells admixed with a few granulocytes; Mild extramedullary hematopoiesis. [2] In a few locally extensive areas, hepatocytes are mild to moderately swollen. [3] Moderate myeloid (predominant), erythroid and megakaryocyte hyperplasia within the red pulp. [4] Mild hypocellularity of the white pulp. [5] Within the mesentery, there are moderate, multifocal infiltrates of macrophages, lymphocytes, plasma cells and granulocytes. [6] Mild to moderate infiltrates of macrophages admixed with lymphocytes, plasma cells and granulocytes; Mild dissociation of the acini and acinar loss; Mild diffuse loss of zymogen granules from the acinar cells. [7] Dense aggregates of lymphocytes admixed with macrophages around fat tissue. [8] Mild multifocal vacuolar degeneration of the chief and parietal cells within the gastric mucosa. (FIG. 5D) Serum chemistry and blood counts. (FIGS. 5E, 5F) Luminex assay measurements of serum IL-6 (FIG. 5E) and TNF-α (FIG. 5F) cytokines 4 or 8 days after a single i.p. injection of IRF5/IKKβ NPs.

FIGS. 6A-61. Intravenously infused IRF5/IKKβ nanoparticles can control tumor metastases in the lung. (FIG. 6A) In vivo biodistribution of macrophage-targeted IRF5/IKKβ NPs following i.v. administration. Codon-optimized mRNA was measured by qPCR 24 hours after a single i.v. injection of particles containing 50 μg mRNA. (FIGS. 6B-6H) C57BL/6 albino mice were injected via tail vein with 1×10⁶ B16F10 firefly luciferase-expressing melanoma cells to establish lung metastases. After 7 days, animals were randomly assigned to either the IRF5/IKKβ NP treatment group, the control GFP NP group, or the PBS control. (FIG. 6B) Time lines and dosing regimens. (FIG. 6C) Confocal microscopy of healthy lungs (left panel) and B16F10 tumor-infiltrated lungs (right panel). Infiltrating macrophage populations fluoresce in green. (FIG. 6D) Sequential bioluminescence tumor imaging. (FIG. 6E) Kaplan-Meier survival curves for each treatment group. ms indicates median survival. Statistical analysis was performed using the log-rank test, and P<0.05 was considered significant. (FIG. 6F) Representative photographs (top row) and micrographs of lungs containing B16F10 melanoma metastases representing each group following 2 weeks of treatment. (FIG. 6G) Counts of lung tumor foci. (FIG. 6H) Phenotypic characterization of monocyte/macrophage populations in bronchoalveolar lavage from each treatment group. (FIG. 6I) Summary of the relative percentages of suppressive and activated macrophages.

FIGS. 7A-7F. Macrophage reprogramming improves the outcome of radiotherapy in glioma. (FIG. 7A) T2 MRI scan, and histological staining following initiation of a PDGFB-driven glioma in RCAS-PDGF-B/Nestin-Tv-a; Ink4a/Arf−/−; Pten−/−transgenic mice on post-induction day 21. (FIG. 7B) Confocal microscopy of CD68+ TAMs infiltrating the glioma margin. Scale bar 300 μm. (FIG. 7C) Flow cytometry analysis of macrophage (F4/80+, CD11b+) populations in healthy brain tissue versus glioma. (FIGS. 7D-7E) Kaplan-Meier survival curves of mice with established gliomas receiving IRF5/IKKβ treatments as a monotherapy (FIG. 7D) or combined with brain tumor radiotherapy (FIG. 7E). Time lines and dosing regimens are shown on top. Ms, median survival. Statistical analysis was performed using the log-rank test, and P<0.05 was considered statistically significant. (FIG. 7F) Sequential bioluminescence imaging of tumor progression.

FIGS. 8A-8E. IVT mRNA-carrying nanoparticles encoding human IRF5/IKKβ efficiently reprogram human macrophages. (FIG. 8A) Time line and culture conditions to differentiate the human THP-1 monocytic cell line into suppressive M2-like macrophages. (FIG. 8B) Bioluminescent imaging of M2-differentiated THP1-Lucia cells cultured in 24 wells and transfected with indicated concentrations of NPs carrying human IRF5/IKKβ mRNA versus control GFP mRNA. Levels of IRF-induced Lucia luciferase were determined 24 hours after transfection using Quanti-Luc. (FIG. 8C) Summary of bioluminescent counts. (FIGS. 8D-8E) Differences in IL-1β cytokine secretion (FIG. 8D) and surface expression (FIG. 8E) of the M1-macrophage marker CD80.

FIG. 9. List of antibodies used in myeloid and lymphoid immunophenotyping panels described in Example 1.

FIG. 10 provides exemplary sequences supporting the disclosure (SEQ ID NOs: 1-44, 110, and 111). The identities of SEQ ID NOs: 1-44, 110, and 111 are described in FIG. 11. Regarding SEQ ID NO: 15 particularly, the fusion protein includes the DBD (DNA binding domain) and CAD (constitutively active domain) of murine IRF7 and the NES (Nuclear Export Signal) and IRF association domains of murine IRF3. The IRF association domain includes Asp mutations at four serine and one threonine residues in the N terminal region, conferring constitutive activation and translocation of the fusion protein (Lin R et al. (1998) supra). Regarding SEQ ID NO: 17 particularly, a SUMO (small ubiquitin-like modifier) binding site in murine IRF8 is at Lysine (K) 310. Binding of SUMO 2/3 prevents IRF-8 from engaging and activating IRF8 responsive genes. Mutation of the K310 residue prevents SUMO binding to IRF8, leading to an increase in IRF8 specific gene transcription 2-5 fold (Chang T-H et al. The Journal of Immunology (2012) 189(7): 3548-3556).

FIG. 11 provides a SEQ ID NO: key of exemplary protein sequences and encoding nucleotide sequences.

### DETAILED DESCRIPTION

A number of adverse physiological conditions are associated with immune system activation (e.g., autoimmune disorders) or immune system suppression (e.g., cancer). For example, macrophages are key immune effector cells that infiltrate cancerous tissue in high numbers. However, within an immunosuppressive tumor milieu, they undergo a switch from an activated tumoricidal state to an immunosuppressive phenotype, which facilitates tumor growth and metastasis. These tumor-associated immunosuppressed macrophages (TAMs) are associated with poor prognosis (Komohara Y et al. (2014) Cancer science 105 (1): 1-8). They induce angiogenesis, lymphogenesis, and stroma remodeling. They also play a key role in promoting tumor invasion and metastasis through secretion of the enzymes plasmin, uPA, matrix metalloproteinases (MMPs) and cathepsin B (Komohara, Y et al. (2016) Advanced drug delivery reviews 99:180-185; Gocheva V et al. (2010) Genes Dev 24:241-255; Wang R et al. (2011) Lung Cancer 74:188-196). Apart from mediating tumor growth and progression, TAMs can also interact with other immune cells and suppress innate and adaptive antitumor immune responses.

Several small molecule drugs focus on blocking the localization of TAM-precursor cells to tumors by targeting the pathways involved in cell recruitment or expansion (i.e. inhibitors of the CSF-1/CSF-1R pathway (Pyon; teck et al. Nat Med 19, 1264-1272 (2013); Tap et al. N Engl J Med 373, 428-437 (2015)) or the CCL2 pathway (Nywening, et al. Lancet Oncol 17, 651-662 (2016)). These approaches require repeated systemic exposure to large doses of the small molecule drugs. Furthermore, clinical trials of these drugs showed low responses unless they were combined with cytoreductive therapies. Nywening, et al. Lancet Oncol 17, 651-662 (2016); Butowski et al. Neuro Oncol 18, 557-564 (2016). Furthermore, these small molecule approaches do not actively promote macrophage anti-tumor activity.

Conventional nanocarriers such as liposomes have been formulated with bisphosphonates or other antiproliferative agents to systemically destroy macrophages within a tumor (i.e. liposomal-clodronate) (Fritz et al., Front Immunol 5, 587 (2014)). Oncolytic viruses have also been used to deliver siRNA to silence immune-evasion pathways within tumors and indirectly promote phagocytosis of TAMs. (Chao et al., Curr Opin Immunol 24, 225-232 (2012)). The macrophages that are destroyed using these approaches, however, are naturally replaced by newly-arriving macrophages that similarly become immunosuppressive.

Antibodies have been developed to induce functional activation of TAMs. These approaches utilize antibodies to target defined antigen types within the tumor. Mantovani, et al., Nat Rev Clin Oncol (2017) Success of these antibodies, however, is limited by their low tumor penetration and heterogeneous distribution. Thurber et al., Adv Drug Deliv Rev 60, 1421-1434 (2008). They also do not address tumor escape variants that lack the antigen targeted by the antibody.

None of the described approaches directly and effectively reprogram TAMs to become activated tumoricidal macrophages, as disclosed herein. The systems and methods disclosed herein are significantly innovative because they allow the reprogramming of TAMs to become tumor-clearing macrophages while simultaneously reducing the tumor-promoting TAM burden. Currently, no other method exists that allow physicians to rationally reprogram TAMs for these therapeutic purposes. Mantovani, et al., Nat Rev Clin Oncol (2017); Gabrilovich & Nagaraj, Nat Rev Immunol 9, 162-174 (2009). This in and of itself can provide therapeutic benefit in the treatment of tumors. By modulating or restructuring the tumor microenvironment, the current disclosure also renders tumors more susceptible to other treatment types, such as vaccines, immunotherapies (e.g., CAR), and/or chemotherapies.

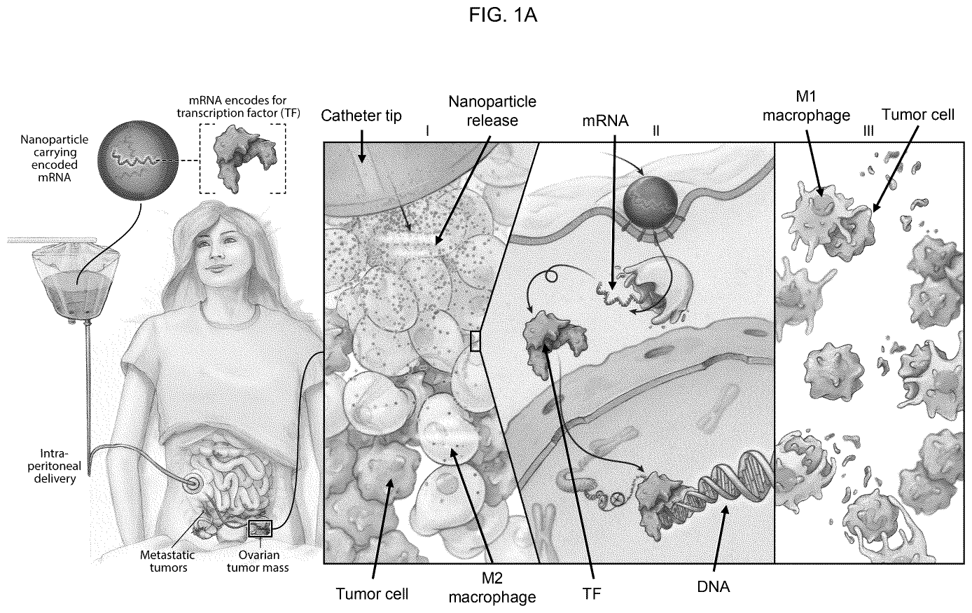

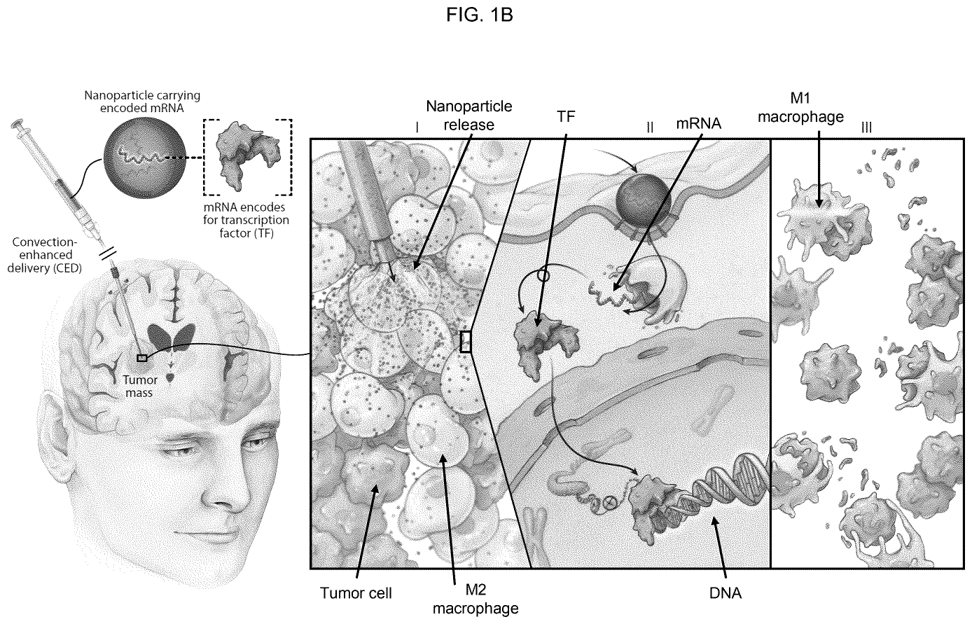

Particular embodiments utilize particles to provide cells with nucleotides encoding genes encoding activation regulators such as transcription factors (e.g., Interferon Regulatory Factors (IRFs)) and/or kinases (e.g., IKKβ). These activation regulators regulate macrophage polarization (FIGS. 1A and 1B). Macrophage polarization is a highly dynamic process through which the physiological activity of macrophages changes. As indicated, in most tumors, TAMs exhibit an immunosuppressed phenotype which can be an “M2” phenotype. By contrast, activated macrophages can exhibit an “M1” phenotype which results in tumor cell killing. Particular embodiments disclosed herein reverse the polarization of tumor-promoting TAMs into tumor-killing macrophages. This effect ameliorates the immunosuppressive milieu within the tumors by inducing inflammatory cytokines, activating other immune cells, and phagocytosing tumor cells.

“Macrophage activation” refers to the process of altering the phenotype or function of a macrophage from (i) an inactivated state to an activated state; (ii) a non-activated state to an activated state; (iii) an activated state to a more activated state; or (iv) an inactivated state to a non-activated state. An inactivated state means an immunosuppressed phenotype that facilitates tumor growth and metastasis. A non-activated state means that the macrophage neither facilitates tumor growth or metastasis nor promotes the killing of tumor cells. Activated means that the macrophage exhibits tumoricidal activity. In particular embodiments, the activated state results in an M1 phenotype as described more fully below. In particular embodiments, the inactivated state results in an M2 phenotype, also as described more fully below.

“Macrophage inactivation” refers to the process of altering the phenotype or function of a macrophage from (i) an activated state to a less activated state; (ii) an activated state to a non-activated state; (iii) an activated state to an inactivated state; or (iv) a non-activated state to an inactivated state. In particular embodiments, the inactivated state is M2. In particular embodiments, the activated state is M1.

In particular embodiments, one benefit of the disclosed systems and methods is that patients can be spared from systemic toxicities because inflammation induced by treatment remains localized at the treatment site. To achieve this benefit, locally infused particles target TAMs in the tumor milieu, (2) deliver nucleotides that selectively reprogram signaling pathways that control macrophage polarization, and (3) are completely degradable locally by physiological pathways (Sahin et al., Nat Rev Drug Discov 13, 759-780 (2014)).

Achieving high expression of exogenous nucleotides in solid tumors is challenging in vivo. Before the current disclosure, nucleotide delivery systems based on viruses or conventional nanocarriers such as liposomes were limited by their restricted diffusion within tumor tissue. Jain & Stylianopoulos, Nat Rev Clin Oncol 7, 653-664 (2010). To circumvent this barrier, particular embodiments utilize nanoparticles (NPs) with enhanced diffusivity so that the NPs deliver nucleotides to a large population of TAMs within a tumor. Particular embodiments utilize NPs <130 nm in size that carry a neutral surface charge. Particular embodiments can further include a targeting ligand attached to the surface of the NP. For example, di-mannose can be attached to the NP surface to enable more selective targeting to the mannose receptor (CD206) expressed on the TAM cell surface. Other TAM cell surface receptors that can be targeted include early growth response protein 2 (Egr2), CD163, CD23, interleukin (IL) 27RA, CLEC4A, CD1a, CD1b, CD93, CD226, IL 13-Ra1, IL-4r, IL-1R type II, decoy IL-1R type II, IL-10r, macrophage scavenging receptors A and B, Ym-1, Ym-2, Low density receptor-related protein 1 (LRP1), IL-6r, CXCR 1/2, and PD-L1.

In particular embodiments, systems and methods disclosed herein include administering particles to a subject in need thereof. The particles are directed to macrophages present in tumors in the subject and are designed to be internalized by the macrophages. Once internalized, the particles further deliver one or more nucleotides having sequences that encode IRF5 and IKKβ. The one or more nucleotides modify the macrophages to express IRF5 and IKKβ. Without being bound by theory, the IKKβ kinase activates the IRF5 transcription factor by phosphorylation. Activated IRF5 then causes expression of type I interferon (IFN) genes, inflammatory cytokines, including tumor necrosis factor (TNF), IL-6, IL-12 and IL-23, and tumor suppressors. In M2 macrophages that have internalized one or more nucleotides encoding IRF5 and IKKβ, the expression of the aforementioned genes through IRF5 action leads to a phenotypic or functional switch of the macrophages from an M2 phenotype to an M1 phenotype, which enables the macrophages to kill or otherwise trigger the destruction of tumor cells, thereby treating cancer. In particular embodiments, the particles are internalized in the macrophages by phagocytosis. In particular embodiments, the particles are internalized in the macrophages by ligand-mediated endocytosis (e.g., CD-206-mediated endocytosis). In particular embodiments, delivery of the particles including the IRF5 and IKKβ genes into macrophages can include, e.g., (1) binding to the macrophages, (2) internalization of the particles by the macrophages, (3) escape from endocytic vesicles into the cytoplasm after internalization, (4) release of the one or more nucleotides, which (5) can be transported into the nucleus of the macrophages and (6) transcribed to deliver genes for expressing IRF5 and IKKβ.

Aspects of the disclosure are now described in more detail as follows: (1) macrophages and macrophage phenotypes; (2) cellular pathways to affect macrophage polarization; (3) nucleotides encoding activation regulators; (4) particles to deliver nucleotides; (5) targeting ligands to more selectively deliver nucleotides; (6) pharmaceutical compositions including particles; (7) methods of use; and (8) experimental examples.

(1) Macrophages and Macrophage Phenotypes. “Macrophage” refers to a white blood cell of the immune system differentiated from bone marrow derived monocytes. Macrophages are characterized by their phagocytic activity and their antigen presentation capacity. Macrophages are key players in both the innate and adaptive immune responses. Phenotypically macrophages express the surface marker F4/80 (Ly71) and may also express other surface markers such as CDIIb (MacI), CDIIc, CD14, CD40 or CD68.

Macrophages play an important role in both innate and adaptive immunity by activating T lymphocytes. In cancer, macrophages are one of the major populations of infiltrating leukocytes associated with solid tumors (Gordon S & Taylor P R (2005) Nature Reviews Immunology 5(12): 953-964). They can be recruited to the tumor site from surrounding tissues or by the tumor itself through the secretion of chemotactic molecules. Macrophages participate in immune responses to tumors in a polarized manner depending on their phenotype. “Phenotype” is used herein to refer to the physical attributes or biochemical characteristics of a cell as a result of the interaction of its genotype and the environment and can include functions of a cell.

Macrophages that activate Th1 T lymphocytes provide an inflammatory response and are often denoted as having an M1-polarized or “classically activated” phenotype. Macrophages in an activated state (i.e. M1 macrophages or macrophages having an M1 phenotype), also referred to as “killer macrophages,” inhibit cell proliferation, cause tissue damage, mediate resistance to pathogens, and possess strong tumoricidal activity. These macrophages can increase expression of mediators that are responsible for antigen presentation and costimulation; promoting infiltration of neutrophils to a tumor area leading to neutrophil-targeted tumor regression. An M1 phenotype can also be evidenced by increased antigen presentation as compared to a relevant control condition. In particular embodiments, an M1 phenotype can be evidenced by M1 macrophage production of reactive oxygen species (ROS) and nitric oxide (NO). NO has anti-proliferative effects integral for protection against pathogens and aberrant cells like tumor cells. In particular embodiments, an M1 phenotype can be evidenced by a pro-inflammatory state that induces Th1 immunity through the production of cytokines such as IL-12. In particular embodiments, macrophages in an activated state are classically activated macrophages that can phagocytose pathogens.

Beyond function, an M1 phenotype can also be evidenced by surface markers expressed by the macrophages; factors, proteins, or compounds produced by the macrophages upon polarization; or genes induced by the macrophages upon polarization. M1 polarization can lead to a phenotype evidenced by expression of CD80, CD86, iNOS, suppressor of cytokine signaling 3 (SOCS3), TNFα, IL-1, IL-6, IL-12, IL-23, Type I IFN, CXCL1, CXCL2, CXCL3, CXCL5, CXCL8, CXCL9, and CXCL10. In particular embodiments, an M1 phenotype includes an increase in expression of CD80. In particular embodiments, an M1 phenotype includes CD206−, MHCII+, CD11c−, and CD11b+.

On the other hand, macrophages that activate Th2 T lymphocytes provide an anti-inflammatory response and are often denoted as having an “M2” phenotype. Macrophages that are in an inactivated state (i.e. M2 macrophages or macrophages having an M2 phenotype), also referred to as “repair macrophages,” are involved in metazoan parasites containment, cell proliferation, tissue repair, tumor progression, anti-inflammation pathways, and immunosuppression. An M2 phenotype can reduce antigen presentation and decrease phagocytosis as compared to a relevant control condition. An M2 phenotype can also be evidenced by, for example, expression of one or more of arginase 1 (Arg1 (arginase activity is associated with pro-proliferative effects and tissue repair responses)), IL-10, TGF-β, PPAry, KLF4, CD206 (MRC1), Dectin-1 (a signaling non-TLR pattern-recognition receptor), DC-SIGN (C-type lectin), scavenger receptor A, scavenger receptor B-1, CD163 (high affinity scavenger receptor for the hemoglobin-haptoglobin complex), chemokine receptors CCR2, CXCR1, and CXCR2, YM1 (chitinase 3—like 3), and Fizz1; and secretion of the chemokines CCL17, CCL22 and CCL24. In particular embodiments, macrophages in an inactivated state promote metastasis and/or resistance to chemotherapy. In particular embodiments, an M2 phenotype includes CD206+, MHCII−, CD11c+, and CD11b^{low}.

Table 1 provides particular combinations of criteria that can be used to distinguish an M1 phenotype from M2 phenotypes (including sub-phenotypes designated as M2a, M2b, M2c and M2d).

TABLE 1
Exemplary Criteria to Categorize Macrophage Phenotypes.

| | M1 | M2a | M2b | M2c | M2d |
|---|---|---|---|---|---|
| Stimulation/ | IFN-γ | IL-4 | ICs | IL-10 | IL-6 |
| Activation | LPS | IL-13 | IL-1R | TGF-β | LIF |
| | GM-CSF | Fungal and | | GCs | Adenosine |
| | | Helminth | | | |
| | | infection | | | |
| Marker | CD86 | CD163 | CD86 | CD163 | VEGF |
| Expression | CD80 | CD23 | MHC II | TLR1 | |
| | CD68 | MHC II | | TLR8 | |
| | MHC II | SR | | | |
| | IL-1R | MMR/CD206 | | | |
| | TLR2 | CD200R | | | |
| | TLR4 | TGM2 | | | |
| | iNOS | DecoyR | | | |
| | SOCS3 | IL-1R II | | | |
| | CD28 | Mouse only: | | | |
| | Gpr18 | Ym1/2 | | | |
| | Fpr2 | Fizz1 | | | |
| | CD64 | Arg-1 | | | |
| Cytokine | TNF | IL-10 | IL-1 | IL-10 | IL-10 |
| secretion | IL-1β | TGF-β | IL-6 | TGF-β | IL-12 |
| | IL-6 | IL-1ra | IL-10 | | TNFα |
| | IL-12 | | TNFα | | TGFβ |
| | IL-23 | | | | |
| Chemokine | CCL10 | CCL17 | CCL1 | CCR2 | CCL5 |
| secretion | CCL11 | CCL22 | | | CXCL10 |
| | CCL5 | CCL24 | | | CXCL16 |
| | CCL8 | | | | |
| | CCL9 | | | | |
| | CCL2 | | | | |
| | CCL3 | | | | |
| | CCL4 | | | | |

Adapted from Röszer T (2015) Mediators Inflamm 2015, 816460 and Duluc D et al. (2007) Blood 110: 4319-4330. Arg-1, arginase-1; Fizz1, resistin-like molecule-alpha (Retnl-alpha); GCs, glucocorticoids; ICs, immune complexes; IL1-ra, IL-1 receptor antagonist; LIF, leukocyte inhibitory factor; TGM2, transglutaminase 2; TGF-β, transforming growth factor-beta; TNFα, tumor necrosis factor alpha; TLR, Toll-like receptor; MMR (CD206), macrophage mannose receptor; iNOS, inducible nitric oxide synthase; SR, scavenger receptor; SOCS3, suppressor of cytokine signaling 3; VEGF, vascular endothelial growth factor; Ym1 (also known as chitinase-3-like protein-3 (Chi3l3)).

Assays to assess macrophage phenotype can take advantage of the different molecular signatures particular to the M1 or M2 phenotype. A commonly accepted marker profile for M1 macrophages is CD80+, whereas M2-macrophages can be characterized as CD163+. Thus, flow cytometry can be performed to assess for these markers. Driving macrophages towards a M1 type and away from a M2 type can also be assessed by measuring an increase of the IL-12/IL-10 ratio or the CD163−/CD163+ macrophage ratio. In particular embodiments, M1 versus M2 morphology can be assessed by light microscopy. In particular embodiments, phagocytosis assays may be used in conjunction with other assays to assess whether a macrophage is M1 type or M2 phenotype. Phagocytosis assays of different macrophage populations may be performed by incubating an entity to be phagocytosed with macrophages at a concentration that is consistent with their normalized total surface area per cell. The entity to be phagocytosed may be added to macrophage cultures. The entity to be phagocytosed may be, for example, labeled with a fluorescent label. Phagocytosis index may be determined by the median total fluorescence intensity measured per macrophage. Quantification of phagocytosis may be by, for example, flow cytometry. Tumor cell killing assays may also be utilized. In particular embodiments, an M1 phenotype includes reduced expression of signature M2 macrophage genes including SerpinB2 (inhibitor of urokinase-type plasminogen activator), CCL2 (C—C motif chemokine ligand 2), CCL11 (C—C motif chemokine ligand 11), and Retnla (resistin like alpha; Fizz1). In particular embodiments, an M1 phenotype includes increased expression of M1 differentiation genes including CCL5 (C—C motif chemokine ligand 5).

Gene expression (e.g., M1 expression of CD80, CD86 and/or other genes noted above) can be measured by assays well known to a skilled artisan. Methods to measure gene expression include NanoString nCounter® expression assays (NanoString Technologies, Inc., Seattle, WA), Northern blots, dot blots, microarrays, serial analysis of gene expression (SAGE), RNA-seq, and quantitative RT-PCR. Methods to measure gene expression products, e.g., protein level, include ELISA (enzyme linked immunosorbent assay), western blot, FACS, radioimmunological assay (RIA), sandwich assay, fluorescent in situ hybridization (FISH), immunohistological staining, immunoelectrophoresis, immunoprecipitation, and immunofluorescence using detection reagents such as an antibody or protein binding agents.

(2) Cellular Pathways to Affect Macrophage Polarization. Polarization of a macrophage towards an activated or inactivated phenotype results from macrophage interaction with a number of different molecules or environments. For example, M1 macrophage polarization is triggered by stimuli including Toll-like receptor (TLR) ligands (e.g. lipopolysaccharide (LPS), muramyl dipeptide, lipoteichoic acid, imiquimod, CpG), IFNγ, TNFα, and macrophage colony-stimulating factor (GM-CSF). M2 polarized macrophages can be divided into subsets, depending on the stimuli that initiates the polarization: the M2a subtype is elicited by IL-4, IL-13 or fungal and helminth infections; M2b is elicited by IL-1 receptor ligands, immune complexes and LPS; M2c is elicited by IL-10, TGF-β and glucocorticoids; and M2d is elicited by IL-6 and adenosine. M2 macrophage polarization may also be triggered by IL-21, GM-CSF, complement components, and apoptotic cells. Macrophage polarization is also modulated by local microenvironmental conditions such as hypoxia.

The aforementioned molecules and environments affect macrophage polarization by triggering different intracellular signaling pathways involving transcription factors. Transcription factors that are involved in both M1 and M2 polarization include IRFs, signal transducers and activators of transcription (STAT), SOCS3 proteins, and nuclear factor kappa-light-chain-enhancer of activated B cells (NFκB). Mitogen-activated protein kinases (MAPK) also play a role in directing macrophage function towards either the M1 or M2 phenotype.

The IRF/STAT pathways, activated by such stimuli as IFNs and TLR signaling as discussed above, polarize macrophages to the M1 activation state via STAT1. On the other hand, such stimuli as IL-4 and IL-13 skew macrophages toward the M2 activation state via STAT6 (Sica A & Bronte V (2007) J Clin Invest 117:1155-1166). These signaling events thus result in either the promotion of an inflammatory immune response and tumoricidal activity, as in the case of M1 macrophage polarization, or in the promotion of an immunosuppressive protumor response, as in the case of M2 macrophage polarization.

Some intracellular molecules implicated in the induction of an M1 phenotype include the G-protein coupled receptor, P2Y (2)R, which plays a role in inducing NO via NOS2 (Eun S Y et al. (2014) Int Immunopharmacol 18:270-276); SOCS3, which activates NFκB/PI-3 kinase pathways to produce NO (Arnold C E et al. (2014) Immunology 141:96-110); and growth and differentiation factor Activin A, which promotes M1 markers and down-regulates IL-10 (Sierra-Filardi E et al. (2011) Blood 117:5092-5101).

Other intracellular molecules involved in induction of the M1 phenotype include IRFs. IRFs are a group of transcription factors with diverse roles, including virus-mediated activation of IFN, and modulation of cell growth, differentiation, apoptosis, and immune system activity. Members of the IRF family are characterized by a conserved N-terminal DNA-binding domain containing tryptophan (W) repeats.

IRF5 is a transcription factor that possesses a helix-turn-helix DNA-binding motif and mediates virus- and IFN-induced signaling pathways. It acts as a molecular switch that controls whether macrophages will promote or inhibit inflammation. IRF5 activates type I IFN genes, inflammatory cytokines, including TNF, IL-6, IL-12 and IL-23, and tumor suppressors as well as Th1 and Th17 responses. It is encoded by the human IRF5 gene located at chromosome 7q32 (OMIM ID 607218). It is appreciated that several isoforms/transcriptional variants of IRF5 exist. In particular embodiments, isoforms of human IRF5 include isoform 1 (UniProt Accession Q13568-1, SEQ ID NO: 1), isoform 2 (UniProt Accession Q13568-2, SEQ ID NO: 2), isoform 3 (UniProt Accession Q13568-3, SEQ ID NO: 3), isoform 4 (UniProt Accession Q13568-4, SEQ ID NO: 4), isoform 5 (UniProt Accession Q13568-5, SEQ ID NO: 5) and isoform 6 (UniProt Accession Q13568-6, SEQ ID NO: 6). In particular embodiments, isoforms of human IRF5 include isoform 1 encoded by a nucleotide sequence shown in SEQ ID NO: 23, isoform 2 encoded by a nucleotide sequence shown in SEQ ID NO: 24, isoform 3 encoded by a nucleotide sequence shown in SEQ ID NO: 25, isoform 4 encoded by a nucleotide sequence shown in SEQ ID NO: 26, isoform 5 encoded by a nucleotide sequence shown in SEQ ID NO: 27 and isoform 6 encoded by a nucleotide sequence shown in SEQ ID NO: 28. In particular embodiments, murine IRF5 includes an amino acid sequence shown in SEQ ID NO: 7. In particular embodiments, murine IRF5 is encoded by a nucleotide sequence shown in SEQ ID NO: 29. M1 macrophages have been shown to upregulate IRF5.

IRF1 and IRF8 also play critical roles in the development and function of myeloid cells, including activation of macrophages by proinflammatory signals such as IFN-γ. Dror N et al. (2007) Mol Immunol. 44 (4): 338-346. In particular embodiments, human IRF1 includes an amino acid sequence shown in SEQ ID NO: 8. In particular embodiments, human IRF1 is encoded by a nucleotide sequence shown in SEQ ID NO: 30. In particular embodiments, murine IRF1 includes an amino acid sequence shown in SEQ ID NO: 12. In particular embodiments, murine IRF1 is encoded by a nucleotide sequence shown in SEQ ID NO: 34. In particular embodiments, human IRF8 includes an amino acid sequence shown in SEQ ID NO: 11. In particular embodiments, human IRF8 is encoded by a nucleotide sequence shown in SEQ ID NO: 33. In particular embodiments, murine IRF8 includes an amino acid sequence shown in SEQ ID NO: 16. In particular embodiments, murine IRF8 is encoded by a nucleotide sequence shown in SEQ ID NO: 38.

IRF3 is a homolog of IRF1 and IRF2. It contains several functional domains including a NES, a DBD, a C-terminal IRF association domain and several regulatory phosphorylation sites. IRF3 is found in an inactive cytoplasmic form that upon serine/threonine phosphorylation forms a complex with CREB Binding Protein, a transcriptional coactivator. This complex translocates to the nucleus and activates the transcription of IFN-α and -β, as well as other interferon-induced genes. In particular embodiments, isoforms of human IRF3 include isoform 1 (UniProt Accession Q14653-1), isoform 2 (UniProt Accession Q14653-2), isoform 3 (UniProt Accession Q14653-3), isoform 4 (UniProt Accession Q14653-4), and isoform 5 (UniProt Accession Q14653-5). In particular embodiments, human IRF3 isoform 1 includes an amino acid sequence shown in SEQ ID NO: 9. In particular embodiments, human IRF3 isoform 1 is encoded by a nucleotide sequence shown in SEQ ID NO: 31. In particular embodiments, murine IRF3 includes an amino acid sequence shown in SEQ ID NO: 13. In particular embodiments, murine IRF3 is encoded by a nucleotide sequence shown in SEQ ID NO: 35.

IRF7 has been shown to play a role in the transcriptional activation of type I IFN genes. In particular embodiments, isoforms of human IRF7 include isoform A (UniProt Accession Q92985-1), isoform B (UniProt Accession Q92985-2), isoform C (UniProt Accession Q92985-3), and isoform D (UniProt Accession Q92985-4). In particular embodiments, human IRF7 isoform A includes an amino acid sequence shown in SEQ ID NO: 10. In particular embodiments, human IRF7 isoform A is encoded by a nucleotide sequence shown in SEQ ID NO: 32. In particular embodiments, murine IRF7 includes an amino acid sequence shown in SEQ ID NO: 14. In particular embodiments, murine IRF7 is encoded by a nucleotide sequence shown in SEQ ID NO: 36.

One or more IRF mutants that contribute to IRF activation may also be used. For example: phosphomimetic mutants of human variant 3/variant 4 of IRF5 (isoform 4, SEQ ID NO: 4) that substitute amino acid residues S425, S427, S430, S436 with residues mimicking phosphorylation, such as aspartic acid residues (Chen W et al. (2008) Nat Struct Mol Biol. 15(11): 1213-1220); phosphomimetic mutants of human variant 5 of IRF5 (isoform 2, SEQ ID NO: 2) that substitute amino acid residues T10, S158, S309, S317, S451, and/or S462 with residues mimicking phosphorylation, such as aspartic acid residues (Chang Foreman H-C et al. infra); mutation of human IRF5 isoform a (variant 1, isoform 3, SEQ ID NO: 3) and isoform b (variant 2, isoform 1, SEQ ID NO: 1) residues S156, S158 and T160 to residues mimicking phosphorylation, such as aspartic acid residues, for constitutive nuclear accumulation of IRF5 (Lin R et al. (2005) J Biol Chem 280 (4): 3088-3095); and IRF3 phosphomimetic mutants that substitute amino acid residue S396 of IRF3 with residues mimicking phosphorylation, such as aspartic acid (Chen Wet al. infra). In particular embodiments, a fusion protein of murine IRF7/IRF3 includes Asp (D) mutations at four serine and one threonine residues in the IRF3 association domains (SEQ ID NO: 15), conferring constitutive activation and translocation of the fusion protein (Lin R et al. (1998) supra; Lin et al. (2000) Molecular and Cellular Biology 20:6342-6353). In particular embodiments, a fusion protein of murine IRF7/IRF3 including D mutations at four serine and one threonine residues in the IRF3 association domains is encoded by a nucleotide sequence shown in SEQ ID NO: 37. In particular embodiments, a murine IRF8 mutant includes substitution of Lysine (K) at amino acid residue 310 with Arginine (R) (SEQ ID NO: 17). In particular embodiments, a murine IRF8 mutant including a substitution of K at amino acid residue 310 with R is encoded by a nucleotide sequence shown in SEQ ID NO: 39. Small ubiquitin-like modifiers (SUMO) bound to IRF8 primarily at K310 inhibit activation of IRF8 responsive genes. Sentrin-specific protease 1 (SENP1) targets SUMO 2/3. The activity of SENP1 “deSUMOylates” IRF8 (and other proteins) and causes IRF8 to go from a repressor of M1 macrophage differentiation to an activator (directly and through transactivation activities). Preventing SUMO binding to IRF8 by mutation of the K310 residue increases IRF8 specific gene transcription 2-5 fold (see Chang T-H et al. (2012) supra).

Particular embodiments of the present disclosure include engineered IRF transcription factors. In particular embodiments, engineered IRF transcription factors include IRFs that lack a functioning autoinhibitory domain and are therefore insensitive to feedback inactivation (Thompson et al. (2018) Front Immunol 9:2622). For example, a human IRF5 with 2-3-fold increase in activity can be obtained by deleting aa 489-539 of the human IRF5 protein (Barnes et al. (2002) Mol Cell Biol 22:5721-5740). In particular embodiments, an autoinhibitory domain of IRF4, a transcription factor that promotes an M2 phenotype, can be deleted or mutated to generate a more active IRF4 in the context of treating an autoimmune disease. In particular embodiments, an autoinhibitory domain of an IRF is found at the carboxy terminus of the IRF protein. In particular embodiments, engineered IRF transcription factors include IRFs that lack one or more functioning nuclear export signals (NES) to entrap IRFs in the nucleus and therefore enhance transcription. For example, nuclear accumulation of human IRF5 can be achieved by mutating the NES of human IRF5 by replacing two leucine residues with alanine (L157A/L159A) (Lin et al. (2000) Molecular and Cellular Biology 20:6342-6353). In particular embodiments, engineered IRF transcription factors include fusions of one or more IRFs, fusions of fragments of one or more IRFs, and fusions of mutated IRFs.

NFκB is also a key transcription factor related to macrophage M1 activation. NFκB regulates the expression of a large number of inflammatory genes including TNFα, IL1B, cyclooxygenase 2 (COX-2), IL-6, and IL12p40. NFκB activity is modulated via the activation of the inhibitor of kappa B kinase (IKK) trimeric complex (two kinases, IKKα, IKKβ, and a regulatory protein, IKKγ). When upstream signals converge at the IKK complex, they first activate IKKβ kinase via phosphorylation, and activated IKKβ further phosphorylates the inhibitory molecule, inhibitor of kappa B (I-κB). This results in the proteosomal degradation of I-κB and the release of NFκB p65/p50 heterodimer from the NFκB/I-κB complex. The NFκB p65/p50 heterodimer is then translocated to the nucleus and binds to the promoters of inflammatory genes.

IKKβ is an activating kinase for NFκB as well as other transcription factors such as IRF5. IKKβ similarly phosphorylates several other signaling pathway components including FOXO3, NCOA3, BCL10, IRS1, NEMO/IKBKG, NFκB subunits RELA and NFκB1, as well as the IKK-related kinases TBK1 and IKBKE. In particular embodiments, isoforms of human IKKβ include isoform 1 (UniProt Accession 014920-1, SEQ ID NO: 18), isoform 2 (UniProt Accession 014920-2 SEQ ID NO: 19), isoform 3 (UniProt Accession 014920-3 SEQ ID NO: 20), and isoform 4 (UniProt Accession 014920-4 SEQ ID NO: 21). In particular embodiments, isoforms of human IKKβ include isoform 1 encoded by a nucleotide sequence shown in SEQ ID NO: 40, isoform 2 encoded by a nucleotide sequence shown in SEQ ID NO: 41, isoform 3 encoded by a nucleotide sequence shown in SEQ ID NO: 42, and isoform 4 encoded by a nucleotide sequence shown in SEQ ID NO: 43. In particular embodiments, murine IKKβ includes an amino acid sequence shown in SEQ ID NO: 22. In particular embodiments, murine IKKβ is encoded by a nucleotide sequence shown in SEQ ID NO: 44.

The present disclosure provides for the co-expression of IRF transcription factors with one or more molecules that can activate the IRFs to effect TAM reprogramming to an activated state for tumor killing. In particular embodiments, co-expression strategies include: co-expression of IRF5 and IKKβ; co-expression of IRF5 and TANK-binding kinase-1 (TBK-1), TNF receptor-associated factor 6 (TRAF6) adaptor, receptor interacting protein 2 (RIP2) kinase, and/or NFκB kinase-ε (IKKε) (Chang Foreman H-C et al. (2012) PLOS One 7 (3): e33098); co-expression of IRF5 and protein kinase DNA-PK (Ryzhakov G et al. (2015) J of Interferon & Cytokine Res 35(2): 71-78); co-expression of IRF5 and protein kinase tyrosine kinase BCR-ABL (Massimo M et al. (2014) Carcinogenesis 35(5): 1132-1143); and co-expression of IRF5 or IRF8 with one or more components of the COP9 signalosome (Korczeniewska J et al. (2013) Mol Cell Biol 33 (6): 1124-1138; Cohen H et al. (2000) J Biol Chem 275(50): 39081-39089).

In particular embodiments, the teachings of the current disclosure can be applied in the management of conditions triggered by hyper-immune activation (e.g., autoimmune diseases). Macrophages play key roles in autoimmune diseases such as systemic lupus erythematosus, multiple sclerosis, rheumatoid arthritis, and Sjögren's syndrome (Ushio et al. World J Immunol 2017; 7(1): 1-8). Thus, cellular pathways that support an immunosuppressive M2 phenotype are also described.

An activation regulator implicated in the induction of the M2 phenotype is Krüppel-like factor 4 (KLF-4). KLF-4 coordinates with STAT6 to induce M2 genes such as Arg-1, CD206 (Mrc1, mannose receptor), Fizz1 (resistin-like a) and peroxisome proliferator-activated receptor gamma (PPAry), and to inhibit M1 genes such as TNFα, Cox-2, CCL5 and iNOS. The nuclear receptor, PPARγ, has been shown to regulate genes involved in oxidative metabolism and activation of the M2 phenotype (Odegaard J I et al. (2007) Nature 447:1116-1120).

The cytokine IL-21 mediates M2 polarization by decreasing NOS2 expression and increasing STAT3 phosphorylation (Li S N et al. (2013) Mediators Inflamm 2013, 548073).

IRF4 negatively regulates TLR signaling in a MyD88 independent manner to drive the M2 phenotype (Satoh T et al. (2010) Nat Immunol 11, 936-944). In particular embodiments, human IRF4 is UniProt Accession Q15306. BMP-7 also induces M2 polarization in vitro via activation of the SMAD-PI3K-Akt-mTOR pathway (Rocher C et al. (2013) Plos One 8: e84009).

Transcription factor glucorticoid-induced leucine zipper (GILZ). GILZ is a dexamethasone-inducible gene that mediates glucocorticoid (GC) actions in a variety of cell types and it can induce the suppressive M2 macrophage phenotype. GILZ expression is rapidly and ubiquitously induced by GCs, and the protein product interacts with known transcription factors, such as NF-κB, Raf-1, TORC2, AP-1, Ras, and C/EBPs, inhibiting the expression of pro-inflammatory genes. Thus, GILZ could mimic the therapeutic anti-inflammatory effects of GCs while avoiding the detrimental ones (Ronchetti, S. et al. Front Endocrinol (Lausanne) 2015; 6:170). In particular embodiments, GILZ is human GILZ of amino acid sequence shown in SEQ ID NO: 110. In particular embodiments, GILZ is human GILZ encoded by a nucleotide sequence shown in SEQ ID NO: 111.

As indicated, hypoxia also influences macrophage polarization through hypoxia inducible factors HIF-1α and HIF-2α. HIF-1α regulates NOS2 expression and supports emergence of an M1 phenotype while HIF-2a regulates Arg1 expression and supports emergence of an M2 phenotype (Takeda N et al. (2010) Genes Dev 24:491-501).

TABLE 2
Signaling molecules and genes involved
in macrophage polarization.

| | M1 | M2 |
|---|---|---|
| Signaling | STAT1alpha/beta | STAT6 |
| Molecules | IRF5 | KLF-4 |
| | Btk | NFκB p50 |
| | P2Y(2)R | homodimers |
| | SOCS3 | PPARγ |
| | Activin A | HIF-2α |
| | HIF1-α | IL-21 |
| | | BMP-7 |
| | | FABP4 |
| | | LXRα |
| Genes | TNFα, Cox-2, | Arg-1, Mrc-1, |
| | CCL5, NOS2 | Fizz1, PPARγ |

Adapted from Sica A and Mantovani A 2012 (supra) and Chávez-Galán L et al. (2015) Front Immunol 6, 253. Arg-1, arginase-1; Fizz1, resistin-like molecule-alpha (Retnl-alpha); STAT, signal transducers and activators of transcription; IRF, interferon regulatory factor; SOCS3, suppressor of cytokine signaling 3; Btk, Bruton's tyrosine kinase; HIF-1α, hypoxia inducible factor 1; KLF-4, Krüppel-like factor 4; TNFα, tumor necrosis factor-alpha; BMP-7, bone morphogenetic protein 7; P2Y(2)R, P2Y purinoceptor 2; PPARγ, peroxisome proliferator-activated receptor γ; NFκB, nuclear factor-kappa B; FABP4, fatty acid binding protein 4; LXRα; liver X receptor alpha.

(3) Nucleotides. Within the current disclosure, nucleotides encoding genes that regulate activation states are delivered to immune cells. “Gene” refers to a nucleotide sequence that encodes an activation regulator. This definition includes various sequence polymorphisms, mutations, and/or sequence variants wherein such alterations do not affect the function of the activation regulator. The term “gene” may include not only coding sequences but also regulatory regions such as promoters, enhancers, and termination regions. The term further can include all introns and other DNA sequences spliced from the mRNA transcript, along with variants resulting from alternative splice sites. Nucleotide sequences encoding the activation regulator can be RNA that directs the expression of the activation regulator. These nucleotide sequences include RNA sequences that are translated, in particular embodiments, into protein. In particular embodiments, one of ordinary skill in the art can appreciate that DNA sequences including thymine (T) bases can be equivalent to mRNA sequences having the same sequence except that T bases are replaced by uracil (U) bases. The nucleotide sequences include both the full-length nucleotide sequences as well as non-full-length sequences derived from the full-length protein. The sequences can also include degenerate codons of the native sequence or sequences that may be introduced to provide codon preference in a specific immune cell. Gene sequences to encode activation regulators described herein are available in publicly available databases and publications. “Encoding” refers to a property of sequences of nucleotides, such as a plasmid, a gene, cDNA, mRNA, to serve as templates for synthesis of an activation regulator.

In particular embodiments, the nucleotides include synthetic mRNA. In particular embodiments, synthetic mRNA is engineered for increased intracellular stability using 5′-capping. Multiple distinct 5′-cap structures can be used to generate the 5′-cap of a synthetic mRNA molecule. For example, the Anti-Reverse Cap Analog (ARCA) cap contains a 5′-5′-triphosphate guanine-guanine linkage where one guanine contains an N7 methyl group as well as a 3′-O-methyl group. Synthetic mRNA molecules may also be capped post-transcriptionally using enzymes responsible for generating 5′-cap structures. For example, recombinant Vaccinia Virus Capping Enzyme and recombinant 2′-O-methyltransferase enzyme can create a canonical 5′-5′-triphosphate linkage between the 5′-most nucleotide of an mRNA and a guanine nucleotide where the guanine contains an N7 methylation and the ultimate 5′-nucleotide contains a 2′-O-methyl generating the Cap1 structure. This results in a cap with higher translational-competency and cellular stability and reduced activation of cellular pro-inflammatory cytokines.

In particular embodiments, other modifications of synthetic mRNA to reduce immunogenicity, promote mRNA stability, and/or promote translation of mRNA can include 5′- and 3′-terminal untranslated regions (UTRs), a Kozak translation initiation sequence in the 5′ UTR, modified ribonucleosides, and/or a polyA tail. In particular embodiments, modified ribonucleosides can include pseudouridine (Ψ), 5-methylcytidine (5mC), N6-methyladenosine (m6A), 2-thiouridine (2sU), 5-methoxyuridine (5moU), and N-1-methylpseudouridine (m14′). In particular embodiments, UTRs can include alpha- and/or beta-globin UTRs. Particular embodiments of producing synthetic mRNA include generating a DNA template containing the coding DNA sequence of the desired protein with a 5′ T₁₀₀₋₂₅₀ overhang by PCR amplification from a corresponding DNA plasmid. The DNA template can then be used to produce the mRNA by an in vitro transcription reaction. During in vitro transcription, a 5′ cap structure (e.g., ARCA), modified ribonucleosides, and/or a 3′ poly(A) tail can be incorporated. A number of in vitro transcription systems are commercially available including from, e.g., MEGAscript T7 transcription kit (ThermoFisher Scientific, Waltham, MA), Riboprobe™ System T7 (Promega, Madison, WI), AmpliScribe™ T7 high yield transcription kit (Epicentre, Madison, WI), and HiScribe™ T7 in vitro transcription kit (New England Biolabs, Ipswich, MA). In particular embodiments, synthetic mRNA can be synthesized by companies that synthesize nucleic acids (e.g., TriLink Biotechnologies, San Diego, CA).

Synthetic mRNA or other nucleotides may be made cyclic. Such nucleotides may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, or 3) ribozyme catalyzed. The newly formed 5′-/3′-linkage may be intramolecular or intermolecular.

In the first route, the 5′-end and the 3′-end of the nucleotide may contain chemically reactive groups that, when close together, form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a nucleotide molecule will undergo a nucleophilic attack on the 5′-NHS-ester moiety forming a new 5′-/3′-amide bond.

In the second route, T4 RNA ligase may be used to enzymatically link a 5′-phosphorylated nucleotide molecule to the 3′-hydroxyl group of a nucleic acid forming a new phosphorodiester linkage. In an example reaction, 1 μg of a nucleic acid molecule can be incubated at 37° C. for 1 hour with 1-10 units of T4 RNA ligase (New England Biolabs, Ipswich, MA) according to the manufacturer's protocol. The ligation reaction may occur in the presence of a split oligonucleotide capable of base-pairing with both the 5′- and 3′-region in juxtaposition to assist the enzymatic ligation reaction.

In the third route, either the 5′- or 3′-end of a cDNA template encodes a ligase ribozyme sequence such that during in vitro transcription, the resultant nucleotide molecule can contain an active ribozyme sequence capable of ligating the 5′-end of a nucleic acid molecule to the 3′-end of a nucleic acid molecule. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In particular embodiments, nucleotides include a plasmid, a cDNA, or an mRNA that can include, e.g., a sequence (e.g., a gene) for expressing an activation regulator. Suitable plasmids include standard plasmid vectors and minicircle plasmids that can be used to transfer a gene to a lymphocyte. The nucleotides (e.g., minicircle plasmids) can further include any additional sequence information to facilitate transient expression in a modified cell. For example, the nucleotides can include promoters, such as general promoters, tissue-specific promoters, cell-specific promoters, and/or promoters specific for the cytoplasm. As indicated, promoters and plasmids (e.g., minicircle plasmids) are generally well known in the art and can be prepared using conventional techniques.

In particular embodiments, a nucleotide encoding an IRF is used in combination with one or more additional nucleotides encoding other IRFs. In particular embodiments, a nucleotide encoding an IRF is used in combination with one or more additional nucleotides encoding other IRFs and with a nucleotide encoding a IKKβ. In particular embodiments, a nucleotide encoding an IRF is used in combination with a nucleotide encoding a IKKβ at a ratio of 0.5:1, 1:1, 2:1, 3:1, 4:1, or 5:1. In particular embodiments, a nucleotide encoding an IRF is used in combination with a nucleotide encoding a IKKβ at a ratio of 3:1.

Particular embodiments can deliver nucleotides within a gene editing system. Gene editing systems modify or affect particular sequences of a cell's endogenous genome. Gene editing systems are useful for targeted genome editing, for example gene disruption, gene editing by homologous recombination, and gene therapy to insert therapeutic genes at the appropriate chromosomal target sites with a human genome.

Particular embodiments utilize transcription activator-like effector nucleases (TALENs) as gene editing systems. TALENs refer to fusion proteins including a transcription activator-like effector (TALE) DNA binding protein and a DNA cleavage domain. TALENs are used to edit genes and genomes by inducing double strand breaks (DSBs) in the DNA, which induce repair mechanisms in cells. Generally, two TALENs must bind and flank each side of the target DNA site for the DNA cleavage domain to dimerize and induce a DSB. The DSB is repaired in the cell by non-homologous end-joining (NHEJ) or by homologous recombination (HR) with an exogenous double-stranded donor DNA fragment.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12th and 13th positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant Fokl endonucleases. The Fokl domain functions as a dimer requiring two constructs with unique DNA binding domains for sites on the target sequence. The Fokl cleavage domain cleaves within a five or six base pair spacer sequence separating the two inverted half-sites.

Particular embodiments utilize MegaTALs as gene editing systems. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments utilize zinc finger nucleases (ZFNs) as gene editing systems. ZFNs are a class of site-specific nucleases engineered to bind and cleave DNA at specific positions. ZFNs are used to introduce DSBs at a specific site in a DNA sequence which enables the ZFNs to target unique sequences within a genome in a variety of different cells. Moreover, subsequent to double-stranded breakage, homologous recombination or non-homologous end joining takes place to repair the DSB, thus enabling genome editing.

ZFNs are synthesized by fusing a zinc finger DNA-binding domain to a DNA cleavage domain. The DNA-binding domain includes three to six zinc finger proteins which are transcription factors. The DNA cleavage domain includes the catalytic domain of, for example, Fokl endonuclease.

Guide RNA can be used, for example, with gene-editing systems such as CRISPR-Cas systems. CRISPR-Cas systems include CRISPR repeats and a set of CRISPR-associated genes (Cas).

In general, any system capable of resulting in functional expression of delivered nucleotides can be used within the current disclosure. However, in particular embodiments, delivery utilizing viral vectors is excluded.

(4) Particles. Particles used within the systems and methods disclosed herein can function to condense and protect nucleotides from enzymatic degradation. Particularly useful materials to use within particles for this purpose include positively charged lipids and/or polymers, including poly(β-amino ester) (PbAE).

Examples of positively charged lipids include esters of phosphatidic acid with an aminoalcohol, such as an ester of dipalmitoyl phosphatidic acid or distearoyl phosphatidic acid with hydroxyethylenediamine. More particular examples of positively charged lipids include 3β-[N--(N′, N′-dimethylaminoethyl) carbamoyl) cholesterol (DC-chol); N,N′-dimethyl-N, N′-dioctacyl ammonium bromide (DDAB); N,N′-dimethyl-N,N′-dioctacyl ammonium chloride (DDAC); 1,2-dioleoyloxypropyl-3-dimethyl-hydroxyethyl ammonium chloride (DORI); 1,2-dioleoyloxy-3-[trimethylammonio]-propane (DOTAP); N-(1-(2,3-dioleyloxy) propyl)-N,N, N-trimethylammonium chloride (DOTMA); dipalmitoylphosphatidylcholine (DPPC); 1,2-dioctadecyloxy-3-[trimethylammonio]-propane (DSTAP); and the cationic lipids described in e.g. Martin et al., Current Pharmaceutical Design 2005, 11, 375-394.

Examples of positively charged polymers that can be used within particles of the current disclosure include polyamines; polyorganic amines (e.g., polyethyleneimine (PEI), polyethyleneimine celluloses); poly(amidoamines) (PAMAM); polyamino acids (e.g., polylysine (PLL), polyarginine); polysaccharides (e.g, cellulose, dextran, DEAE dextran, starch); spermine, spermidine, poly(vinylbenzyl trialkyl ammonium), poly(4-vinyl-N-alkyl-pyridiumiun), poly(acryloyl-trialkyl ammonium), and Tat proteins.

Blends of lipids and polymers in any concentration and in any ratio can also be used. Blending different polymer types in different ratios using various grades can result in characteristics that borrow from each of the contributing polymers. Various terminal group chemistries can also be adopted.

Particular embodiments disclosed herein can also utilize porous particles constructed from any material capable of forming a porous network. Exemplary materials include metals, transition metals and metalloids. Exemplary metals, transition metals and metalloids include lithium, magnesium, zinc, aluminum and silica. In particular embodiments, the porous particles include silica. The exceptionally high surface area of mesoporous silica (exceeding 1,000 m2/g) enables nucleotide loading at levels exceeding conventional DNA carriers such as liposomes.

Particles can be formed in a variety of different shapes, including spheroidal, cuboidal, pyramidal, oblong, cylindrical, toroidal, and the like. The nucleotides can be included in the pores of the particles in a variety of ways. For example, the nucleotides can be encapsulated in the porous particles. In other aspects, the nucleotides can be associated (e.g., covalently and/or non-covalently) with the surface or close underlying vicinity of the surface of the porous particles. In particular embodiments, the nucleotides can be incorporated in the porous particles e.g., integrated in the material of the porous particles. For example, the nucleotides can be incorporated into a polymer matrix of polymer particles.

In particular embodiments, the particles disclosed herein include a coating. A coating can serve to shield the encapsulated nucleotides and/or reduce or prevent off-target binding. Off-target binding is reduced or prevented by reducing the surface charge of the particles to neutral or negative. As disclosed in more detail elsewhere herein, coatings can include neutral or negatively charged polymer- and/or liposome-based coatings. In particular embodiments, the coating is a dense surface coating of hydrophilic and/or neutrally charged hydrophilic polymer sufficient to prevent the encapsulated nucleotides from being exposed to the environment before release into an immune cell. In particular embodiments, the coating covers at least 80% or at least 90% of the surface of the particle. In particular embodiments, the coating includes polyglutamic acid (PGA). In particular embodiments, PGA can serve as a linker to attach a targeting ligand to a particle. In particular embodiments, PGA can serve as a linker to attach di-mannose to a particle. In particular embodiments, the coating includes hyaluronic acid.

Examples of neutrally charged polymers that can be used as coating within embodiments of the disclosure include polyethylene glycol (PEG); poly(propylene glycol); and polyalkylene oxide copolymers, (PLURONIC®, BASF Corp., Mount Olive, NJ).

Neutrally charged polymers also include zwitterionic polymers. Zwitterionic refers to the property of overall charge neutrality while having both a positive and a negative electrical charge. Zwitterionic polymers can behave like regions of cell membranes that resist cell and protein adhesion.

Zwitterionic polymers include zwitterionic constitutional units including pendant groups (i.e., groups pendant from the polymer backbone) with zwitterionic groups. Exemplary zwitterionic pendant groups include carboxybetaine groups (e.g., —Ra—N+(Rb)(Rc)-Rd-CO₂—, where Ra is a linker group that covalently couples the polymer backbone to the cationic nitrogen center of the carboxybetaine groups, Rb and Rc are nitrogen substituents, and Rd is a linker group that covalently couples the cationic nitrogen center to the carboxy group of the carboxybetaine group).

Examples of negatively charged polymers include alginic acids; carboxylic acid polysaccharides; carboxymethyl cellulose; carboxymethyl cellulose-cysteine; carrageenan (e.g., GELCARIN® 209, GELCARIN® 379, FMC Corporation, Philadelphia, PA); chondroitin sulfate; glycosaminoglycans; mucopolysaccharides; negatively charged polysaccharides (e.g., dextran sulfate); poly(acrylic acid); poly(D-aspartic acid); poly(L-aspartic acid); poly(L-aspartic acid) sodium salt; poly(D-glutamic acid); poly(L-glutamic acid); poly(L-glutamic acid) sodium salt; poly(methacrylic acid); sodium alginate (e.g., PROTANAL® LF 120M, PROTANAL® LF 200M, PROTANAL® LF 200D, FMC Biopolymer Corp., Drammen, Norway); sodium carboxymethyl cellulose (CMC); sulfated polysaccharides (heparins, agaropectins); pectin, gelatin and hyaluronic acid.

In particular embodiments, polymers disclosed herein can include “star shaped polymers,” which refer to branched polymers in which two or more polymer branches extend from a core. The core is a group of atoms having two or more functional groups from which the branches can be extended by polymerization. In particular embodiments, nanoparticles of the present disclosure include star shaped polymers. In particular embodiments, nanoparticles of the present disclosure include star shaped polymers and a coating. In particular embodiments, nanoparticles of the present disclosure include star shaped polymers and a coating including PGA. In particular embodiments, nanoparticles of the present disclosure include star shaped polymers and a coating including hyaluronic acid.

In particular embodiments, the branches are zwitterionic or negatively-charged polymeric branches. For star polymers, the branch precursors can be converted to zwitterionic or negatively-charged polymers via hydrolysis, ultraviolet irradiation, or heat. The polymers also may be obtained by any polymerization method effective for polymerization of unsaturated monomers, including atom transfer radical polymerization (ATRP), reversible addition-fragmentation chain transfer polymerization (RAFT), photo-polymerization, ring-opening polymerization (ROP), condensation, Michael addition, branch generation/propagation reaction, or other reactions.

Liposomes are microscopic vesicles including at least one concentric lipid bilayer. Vesicle-forming lipids are selected to achieve a specified degree of fluidity or rigidity of the final complex. In particular embodiments, liposomes provide a lipid composition that is an outer layer surrounding a porous particle. In particular embodiments, nanoparticles of the present disclosure include liposomal nanoparticles.

Liposomes can be neutral (cholesterol) or bipolar and include phospholipids, such as phosphatidylcholine (PC), phosphatidylethanolamine (PE), phosphatidylinositol (PI), and sphingomyelin (SM) and other type of bipolar lipids including dioleoylphosphatidylethanolamine (DOPE), with a hydrocarbon chain length in the range of 14-22, and saturated or with one or more double C═C bonds. Examples of lipids capable of producing a stable liposome, alone, or in combination with other lipid components are phospholipids, such as hydrogenated soy phosphatidylcholine (HSPC), lecithin, phosphatidylethanolamine, lysolecithin, lysophosphatidylethanolamine, phosphatidylserine, phosphatidylinositol, sphingomyelin, cephalin, cardiolipin, phosphatidic acid, cerebro sides, distearoylphosphatidylethanolamine (DSPE), dioleoylphosphatidylcholine (DOPC), dipalmitoylphosphatidylcholine (DPPC), palmitoyloleoylphosphatidylcholine (POPC), palmitoyloleoylphosphatidylethanolamine (POPE) and dioleoylphosphatidylethanolamine 4-(N-maleimido-methyl)cyclohexane-1-carboxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stereate, amphoteric acrylic polymers, polyethyloxylated fatty acid amides, DDAB, dioctadecyl dimethyl ammonium chloride (DODAC), 1,2-dimyristoyl-3-trimethylammonium propane (DMTAP), DOTAP, DOTMA, DC—Chol, phosphatidic acid (PA), dipalmitoylphosphatidylglycerol (DPPG), dioleoylphosphatidylglycerol, DOPG, and dicetylphosphate. In particular embodiments, lipids used to create liposomes disclosed herein include cholesterol, hydrogenated soy phosphatidylcholine (HSPC) and, the derivatized vesicle-forming lipid PEG-DSPE.

Methods of forming liposomes are described in, for example, U.S. Pat. Nos. 4,229,360; 4,224,179; 4,241,046; 4,737,323; 4,078,052; 4,235,871; 4,501,728; and 4,837,028, as well as in Szoka et al., Ann. Rev. Biophys. Bioeng. 9:467 (1980) and Hope et al., Chem. Phys. Lip. 40:89 (1986).

The size of particles can vary over a wide range and can be measured in different ways. In preferred embodiments, the particles are NPs <130 nm in size. However, NPs of the present disclosure can also have a minimum dimension of equal to or less than 500 nm, less than 150 nm, less than 140 nm, less than 120 nm, less than 110 nm, less than 100 nm, less than 90 nm, less than 80 nm, less than 70 nm, less than 60 nm, less than 50 nm, less than 40 nm, less than 30 nm, less than 20 nm, or less than 10 nm. In particular embodiments, the particles are NPs 90 to 130 nm in size.

In particular embodiments, the NPs can have a minimum dimension ranging between 5 nm and 500 nm, between 10 nm and 100 nm, between 20 nm and 90 nm, between 30 nm and 80 nm, between 40 nm and 70 nm, and between 40 nm and 60 nm. In particular embodiments, the dimension is the diameter of NPs or coated NPs. In particular embodiments, a population of particles of the present disclosure can have a mean minimum dimension of equal to or less than 500 nm, less than 100 nm, less than 90 nm, less than 80 nm, less than 70 nm, less than 60 nm, less than 50 nm, less than 40 nm, less than 30 nm, less than 20 nm, or less than 10 nm. In particular embodiments, a population of NPs in a composition of the present disclosure can have a mean diameter ranging between 5 nm and 500 nm, between 10 nm and 100 nm, between 20 nm and 90 nm, between 30 nm and 80 nm, between 40 nm and 70 nm, and between 40 nm and 60 nm, between 70 nm and 130 nm or between 75 nm and 125 nm. Dimensions of the particles can be determined using, e.g., conventional techniques, such as dynamic light scattering and/or electron microscopy. While not preferred, in particular embodiments, microparticles could also be used.

In particular embodiments, PbAE polymers are mixed with nucleotides (e.g., in vitro transcribed mRNA) in a ratio of 20:1, 30:1, 40:1, 50:1, 60:1, 70:1, 80:1, 90:1, 100:1, or more to generate PbAE-nucleotide polyplexes. In particular embodiments, PbAE polymers are mixed with nucleotides (e.g., in vitro transcribed mRNA) in a ratio of 60:1 to generate PbAE-nucleotide polyplexes. In particular embodiments, the PbAE-nucleotide polyplexes can be combined with PGA/Di-mannose to form the final NPs.

(5) Targeting Ligands. Targeting ligands can be used on the surface of particles and can lead to more selective binding of immune cells of interest within a heterogeneous cell population.

In particular embodiments, targeting ligands include binding domains derived from cell marker ligands, receptor ligands, antibodies, peptides, peptide aptamers, nucleic acids, nucleic acid aptamers, spiegelmers or combinations thereof. In particular embodiments, within the context of cell targeting ligands, binding domains include any substance that binds to another substance to form a complex capable of mediating endocytosis.

In particular embodiments, binding domains are derived from antibodies. Binding domains derived from antibodies can include whole antibodies or can include binding fragments of an antibody, e.g., Fv, Fab, Fab′, F(ab′)2, Fc, and single chain Fv fragments (scFvs) or any biologically effective fragments of an immunoglobulin that bind specifically to a targeted motif expressed by an immune cell. Antibodies or antigen binding fragments include all or a portion of polyclonal antibodies, monoclonal antibodies, human antibodies, humanized antibodies, synthetic antibodies, chimeric antibodies, bispecific antibodies, mini bodies, and linear antibodies.

Antibodies from human origin or humanized antibodies have lowered or no immunogenicity in humans and have a lower number of non-immunogenic epitopes compared to non-human antibodies. Antibodies and their fragments will generally be selected to have a reduced level or no antigenicity in human subjects.

Antibodies that specifically bind a motif expressed by an immune cell can be prepared using methods of obtaining monoclonal antibodies, methods of phage display, methods to generate human or humanized antibodies, or methods using a transgenic animal or plant engineered to produce antibodies as is known to those of ordinary skill in the art (see, for example, U.S. Pat. Nos. 6,291,161 and 6,291,158). Phage display libraries of partially or fully synthetic antibodies are available and can be screened for an antibody or fragment thereof that can bind to an immune cell motif. For example, binding domains may be identified by screening a Fab phage library for Fab fragments that specifically bind to a target of interest (see Hoet et al., Nat. Biotechnol. 23:344, 2005). Phage display libraries of human antibodies are also available. Additionally, traditional strategies for hybridoma development using a target of interest as an immunogen in convenient systems (e.g., mice, HuMAb mouse®, TC mouse™, KM-mouse®, llamas, chicken, rats, hamsters, rabbits, etc.) can be used to develop binding domains. In particular embodiments, antibodies specifically bind to motifs expressed by a selected immune cell and do not cross react with nonspecific components or unrelated targets. Once identified, the amino acid sequence or nucleotide sequence coding for the antibody can be isolated and/or determined.

An intact antibody can include at least two heavy (H) chains and two light (L) chains inter-connected by disulfide bonds. Each heavy chain is composed of a heavy chain variable region (abbreviated herein as VH or V_{H}) and a heavy chain constant region. The heavy chain constant region includes three domains, CH1, CH2 and CH3. Each light chain is composed of a light chain variable region (abbreviated herein as VL or V_{L}) and a light chain constant region. The light chain constant region includes one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. In particular embodiments, an antibody includes antigen-binding portions of an intact antibody that retain capacity to bind. Examples of fragments that retain capacity to bind include: (i) an Fab fragment, a monovalent fragment including the VL, VH, CL and CH1 domains; (ii) a F(ab′)₂ fragment, a bivalent fragment including two Fab fragments linked by a disulfide bridge at the hinge region; (iii) an Fd fragment including the VH and CH1 domains; (iv) an Fv fragment including the VL and VH domains of a single arm of an antibody, (v) a dAb fragment (Ward et al., Nature, 341:544-546 (1989)), including a VH domain; and (vi) an isolated complementarity determining region (CDR).

The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); Al-Lazikani et al. (1997) J Mol Biol 273:927-948 (Chothia numbering scheme); Maccallum et al. (1996) J Mol Biol 262:732-745 (Contact numbering scheme); Martin et al. (1989) Proc. Natl. Acad. Sci., 86:9268-9272 (AbM numbering scheme); Lefranc M P et al. (2003) Dev Comp Immunol 27 (1): 55-77 (IMGT numbering scheme); and Honegger and Pluckthun (2001) J Mol Biol 309 (3): 657-670 (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In particular embodiments, the antibody CDR sequences disclosed herein are according to Kabat numbering. In particular embodiments, CDR regions are found within antibody regions as numbered by Kabat as follows: for the light chain: CDRL1 are amino acids 24-34; CDRL2 are amino acids 50-56; CDRL3 are amino acids 89-97 and for the heavy chain: CDRH1 are amino acids 31-35; CDRH2 are amino acids 50-65; and CDRH3 are amino acids 95-102.

Peptide aptamers include a peptide loop (which is specific for a target protein) attached at both ends to a protein scaffold. This double structural constraint greatly increases the binding affinity of the peptide aptamer to levels comparable to an antibody. The variable loop length is typically 8 to 20 amino acids (e.g., 8 to 12 amino acids), and the scaffold may be any protein which is stable, soluble, small, and non-toxic (e.g., thioredoxin-A, stefin A triple mutant, green fluorescent protein, eglin C, and cellular transcription factor Spl). Peptide aptamer selection can be made using different systems, such as the yeast two-hybrid system (e.g., Gal4 yeast-two-hybrid system) or the LexA interaction trap system.

Nucleic acid aptamers are single-stranded nucleotide sequence (DNA or RNA) ligands that function by folding into a specific globular structure that dictates binding to target proteins or other molecules with high affinity and specificity, as described by Osborne et al., Curr. Opin. Chem. Biol. 1:5-9, 1997; and Cerchia et al., FEBS Letters 528:12-16, 2002. In particular embodiments, aptamers are small (15 KD; or between 15-80 nucleotides or between 20-50 nucleotides). Aptamers are generally isolated from libraries including 1014-1015 random oligonucleotide sequences by a procedure termed SELEX (see, for example, Tuerk et al., Science, 249:505-510, 1990; Green et al., Methods Enzymology. 75-86, 1991; and Gold et al., Annu. Rev. Biochem., 64:763-797, 1995). Further methods of generating aptamers are described in, for example, U.S. Pat. Nos. 6,344,318; 6,331,398; 6,110,900; 5,817,785; 5,756,291; 5,696,249; 5,670,637; 5,637,461; 5,595,877; 5,527,894; 5,496,938; 5,475,096; and 5,270,16. Spiegelmers are similar to nucleic acid aptamers except that at least one β-ribose unit is replaced by β-D-deoxyribose or a modified sugar unit selected from, for example, β-D-ribose, α-D-ribose, β-L-ribose.

In particular embodiments, targeted cells are TAMs. Targeted cells could also include regulatory T cells (TREG). TREG are a subpopulation of T cells, which modulate the immune system, maintain tolerance to self-antigens, and abrogate autoimmune disease. TREG express CD25, CTLA-4, GITR, GARP and LAP. Selected cell targeting ligands disclosed herein can bind CD25, CTLA-4, GITR, GARP and/or LAP to achieve selective delivery of nucleotides to naïve TREG. Other cell types that can be targeted include myeloid-derived suppressor cells (MDSC), regulatory dendritic cells (DCreg), neutrophils, T helper 17 cells (Th17), regulatory B cells (Breg), and/or mesenchymal stromal cells (MSC). One of ordinary skill in the art can identify appropriate cellular markers to target these cell types utilizing targeting ligands as disclosed herein.

M2 Binding Domains. In particular embodiments, Egr2 is targeted on M2 macrophages. Commercially available antibodies for Egr2 can be obtained from Thermo Fisher, Waltham, MA; Abcam, Cambridge, MA; Millipore Sigma, Burlington, MA; Miltenyi Biotec, Bergisch Gladbach, Germany; LifeSpan Biosciences, Inc., Seattle, WA; and Novus Biologicals, Littleton, CO. Generation of anti-Egr2 antibodies are discussed, for example, in Murakami K et al. (1993) Oncogene 8 (6): 1559-1566. Anti-Egr2 antibodies include: rabbit monoclonal anti-Egr2 antibody clone EPR4004; mouse monoclonal anti-Egr2 antibody clone 1G5; mouse monoclonal anti-Egr2 antibody clone OTI1B12; rabbit polyclonal anti-Egr2 antibody recognizing AA residues 200-300 of human Egr2; rabbit polyclonal anti-Egr2 antibody recognizing AA residues 340-420 of human Egr2; and rabbit polyclonal anti-Egr2 antibody recognizing AA residues 370-420 of human Egr2. Binding domains can be derived from these antibodies and other antibodies disclosed herein.

In particular embodiments, the targeting ligand includes a human or humanized binding domain (e.g., nanobody) including a variable heavy chain including a CDRH1 sequence including SGNIFSINAIG (SEQ ID NO: 45), a CDRH2 sequence including TITLSGSTN (SEQ ID NO: 46), and a CDRH3 sequence including NTYSDSDVYGY (SEQ ID NO: 47). These reflect CDR sequences that bind CD206.

In particular embodiments, the targeting ligand includes a human or humanized binding domain (e.g., nanobody) including a variable heavy chain including a CDRH1 sequence including PGFKLDYYAIA (SEQ ID NO: 48), a CDRH2 sequence including SINSSGGST (SEQ ID NO: 49), and a CDRH3 sequence including LRRYYGLNLDPGSYDY (SEQ ID NO: 50). These reflect CDR sequences that bind CD206.

In particular embodiments, the targeting ligand includes a human or humanized binding domain (e.g., nanobody) including a variable heavy chain including a CDRH1 sequence including GFPFNIYPMS (SEQ ID NO: 51), a CDRH2 sequence including YISHGGTTT (SEQ ID NO: 52), and a CDRH3 sequence including GYARLMTDSELV (SEQ ID NO: 53). These reflect CDR sequences that bind CD206.

A number of additional antibodies specific for CD206 are known to those of skill in the art and can be readily characterized for sequence, epitope binding, and affinity. See, for example, WO 2014/140376, WO 2013/174537, and U.S. Pat. No. 7,560,534. Commercially available antibodies for CD206 can be obtained from Thermo Fisher, Waltham, MA; Proteintech, Rosemont, IL; BioLegend, San Diego, CA; R & D Systems, Minneapolis, MN; LifeSpan Biosciences, Inc., Seattle, WA; Novus Biologicals, Littleton, CO; and Bio-Rad, Hercules, CA. In particular embodiments, an anti-CD206 antibody includes a rat monoclonal anti-mouse CD206 monoclonal antibody clone C068C2 (Cat #141732, Biolegend, San Diego, CA).

In particular embodiments, the targeting ligand includes a human or humanized binding domain (e.g., scfv) including a variable light chain including a CDRL1 sequence including ASQSVSHDV (SEQ ID NO: 54), a CDRL2 sequence including YTS, and a CDRL3 sequence including QDYSSPRT (SEQ ID NO: 56). In particular embodiments, the targeting ligand includes a human or humanized binding domain (e.g., scfv) including a variable heavy chain including a CDRH1 sequence including GYSITSDY (SEQ ID NO: 57), a CDRH2 sequence including YSG, and a CDRH3 sequence including CVSGTYYFDYWG (SEQ ID NO: 59). These reflect CDR sequences of the Mac2-48 antibody that bind CD163.

In particular embodiments, the targeting ligand includes a human or humanized binding domain (e.g., scfv) including a variable light chain including a CDRL1 sequence including ASQSVSSDV (SEQ ID NO: 60), a CDRL2 sequence including YAS, and a CDRL3 sequence including QDYTSPRT (SEQ ID NO: 62). In particular embodiments, the targeting ligand includes a human or humanized binding domain (e.g., scfv) including a variable heavy chain including a CDRH1 sequence including GYSITSDY (SEQ ID NO: 63), a CDRH2 sequence including YSG, and a CDRH3 sequence including CVSGTYYFDYWG (SEQ ID NO: 65). These reflect CDR sequences of the Mac2-158 antibody that bind CD163.

A number of additional antibodies or binding domains specific for CD163 are known to those of skill in the art and can be readily characterized for sequence, epitope binding, and affinity. See, for example, WO 2011/039510, WO 2002/032941, WO 2002/076501, and US 2005/0214871. Commercially available antibodies for CD163 can be obtained from Thermo Fisher, Waltham, MA; Enzo Life Sciences, Inc., Farmingdale, NY; BioLegend, San Diego, CA; R & D Systems, Minneapolis, MN; LifeSpan Biosciences, Inc., Seattle, WA; and RDI Research Diagnostics, Flanders, NJ. In particular embodiments, anti-CD163 antibodies can include: mouse monoclonal anti-CD163 antibody clone 3D4; mouse monoclonal anti-CD163 antibody clone Ber-Mac3; mouse monoclonal anti-CD163 antibody clone EDHu-1; and mouse monoclonal anti-CD163 antibody clone GHI/61.

In particular embodiments, the targeting ligand includes a human or humanized binding domain (e.g., scfv) including a variable light chain including a CDRL1 sequence including RSSKSLLYKDGKTYLN (SEQ ID NO: 66), a CDRL2 sequence including LMSTRAS (SEQ ID NO: 67), and a CDRL3 sequence including QQLVEYPFT (SEQ ID NO: 68). In particular embodiments, the targeting ligand includes a human or humanized binding domain (e.g., scfv) including a variable heavy chain including a CDRH1 sequence including GYWMS (SEQ ID NO: 69), a CDRH2 sequence including EIRLKSDNYATHYAESVKG (SEQ ID NO: 70), and a CDRH3 sequence including FID. These reflect CDR sequences that bind CD23.

A number of antibodies or binding domains specific for CD23 are known to those of skill in the art and can be readily characterized for sequence, epitope binding, and affinity. See, for example, U.S. Pat. Nos. 7,008,623, 6,011,138 A (antibodies including 5E8, 6G5, 2C8, B3B1 and 3G12), US 2009/0252725, Rector et al. (1985) J. Immunol. 55:481-488; Flores-Rumeo et al. (1993) Science 241:1038-1046; Sherr et al. (1989) J. Immunol. 142:481-489; and Pene et al., (1988) PNAS 85:6820-6824. Commercially available antibodies for CD23 can be obtained from Thermo Fisher, Waltham, MA; Abcam, Cambridge, MA; Bioss Antibodies, Inc., Woburn, MA; Bio-Rad, Hercules, CA; LifeSpan Biosciences, Inc., Seattle, WA; and Boster Biological Technology, Pleasanton, CA. In particular embodiments, anti-CD23 antibodies can include: mouse monoclonal anti-CD23 antibody clone Tu 1; rabbit monoclonal anti-CD23 antibody clone SP23; rabbit monoclonal anti-CD23 antibody clone EPR3617; mouse monoclonal anti-CD23 antibody clone 5B5; mouse monoclonal anti-CD23 antibody clone 1B12; mouse monoclonal anti-CD23 antibody clone M-L23.4; and mouse monoclonal anti-CD23 antibody clone 3A2.

M1 Binding Domains. In particular embodiments, the targeting ligand includes a human or humanized binding domain (e.g., scfv) including a variable light chain including a CDRL1 sequence including SSNIGDNY (SEQ ID NO: 72), a CDRL2 sequence including RDS, and a CDRL3 sequence including QSYDSSLSGS (SEQ ID NO: 74). In particular embodiments, the targeting ligand includes a human or humanized binding domain (e.g., scfv) including a variable heavy chain including a CDRH1 sequence including GFTFDDYG (SEQ ID NO: 75), a CDRH2 sequence including ISWNGGKT (SEQ ID NO: 76), and a CDRH3 sequence including ARGSLFHDSSGFYFGH (SEQ ID NO: 77). These reflect CDR sequences of the Ab79 antibody that bind CD38.

In particular embodiments, the targeting ligand includes a human or humanized binding domain (e.g., scfv) including a variable light chain including a CDRL1 sequence including NSNIGSNT (SEQ ID NO: 78), a CDRL2 sequence including SDS, and a CDRL3 sequence including QSYDSSLSGSR (SEQ ID NO: 80). In particular embodiments, the targeting ligand includes a human or humanized binding domain (e.g., scfv) including a variable heavy chain including a CDRH1 sequence including GFTFNNYG (SEQ ID NO: 81), a CDRH2 sequence including ISYDGSDK (SEQ ID NO: 82), and a CDRH3 sequence including ARVYYYGFSGPSMDV (SEQ ID NO: 83). These reflect CDR sequences of the Ab19 antibody that bind CD38.

In particular embodiments, the targeting ligand includes a human or humanized binding domain (e.g., scfv) including a variable light chain including a CDRL1 sequence including RASQSVSSYLA (SEQ ID NO: 84), a CDRL2 sequence including DASNRAT (SEQ ID NO: 85), and a CDRL3 sequence including QQRSNWPPTF (SEQ ID NO: 86). In particular embodiments, the targeting ligand includes a human or humanized binding domain (e.g., scfv) including a variable heavy chain including a CDRH1 sequence including SFAMS (SEQ ID NO: 87), a CDRH2 sequence including AISGSGGGTYYADSVKG (SEQ ID NO: 88), and a CDRH3 sequence including DKILWFGEPVFDY (SEQ ID NO: 89). These reflect CDR sequences of the daratumumab antibody that bind CD38 described in U.S. Pat. No. 7,829,693.

A number of antibodies specific for CD38 are known to those of skill in the art and can be readily characterized for sequence, epitope binding, and affinity. See, for example, WO 2005/103083, WO 2006/125640, WO 2007/042309, WO 2008/047242, WO 2012/092612, WO 2006/099875, WO 2011/154453, WO 2015/130728, U.S. Pat. No. 7,829,693, and US 2016/0200828. Commercially available antibodies for CD38 can be obtained from Thermo Fisher, Waltham, MA; Abcam, Cambridge, MA; and Millipore Sigma, Burlington, MA. In particular embodiments, anti-CD23 antibodies can include: rabbit monoclonal anti-CD38 antibody clone GAD-3; mouse monoclonal anti-CD38 antibody clone HIT2; mouse monoclonal anti-CD38 antibody clone AT1; mouse monoclonal anti-CD38 antibody clone AT13/5; rat monoclonal anti-CD38 antibody clone NIMR-5; and rat monoclonal IgG2a, K anti-CD38 antibody clone 90/CD38 (Cat #BD Biosciences, San Jose, CA).

In particular embodiments, G-protein coupled receptor 18 (Gpr18) is targeted on M1 macrophages. Commercially available antibodies for Gpr18 can be obtained from Assay Biotechnology Company Inc., Sunnyvale, CA; Thermo Fisher, Waltham, MA; Abcam, Cambridge, MA; GeneTex, Inc., Irvine, CA; and Novus Biologicals, Littleton, CO. In particular embodiments, anti-Gpr18 antibodies include: rabbit polyclonal anti-Gpr18 antibody recognizing a portion of amino acids 1-50 of human Gpr18; rabbit polyclonal anti-Gpr18 antibody recognizing a region including amino acids 160-240 of human Gpr18; rabbit polyclonal anti-Gpr18 antibody recognizing a region including amino acids 100-180 of human Gpr18; rabbit monoclonal anti-Gpr18 antibody clone EPR12359; and rabbit polyclonal anti-Gpr18 antibody recognizing a region including amino acids 140-190 of human Gpr18.

In particular embodiments, formyl peptide receptor 2 (Fpr2) is targeted on M1 macrophages. Commercially available antibodies for Fpr2 can be obtained from Atlas Antibodies, Bromma, Sweden; Biorbyt, LLC, San Francisco, CA; Cloud-Clone Corp., Katy, TX; US Biological Life Sciences, Salem, MA; and Novus Biologicals, Littleton, CO. In particular embodiments, anti-fpr2 antibodies include: mouse monoclonal anti-fpr2 antibody clone GM1D6; mouse monoclonal anti-fpr2 antibody clone 304405; recombinant anti-fpr2 antibody clone REA663; and rabbit polyclonal anti-fpr2 antibody recognizing a region including amino acids 300-350 of fpr2.

In particular embodiments, the targeting ligand includes a human or humanized binding domain (e.g., scfv) including a variable light chain including a CDRL1 sequence including RASQSVSSYLA (SEQ ID NO: 90), a CDRL2 sequence including DASSRAT (SEQ ID NO: 91), and a CDRL3 sequence including QLRSNWPPYT (SEQ ID NO: 92). In particular embodiments, the targeting ligand includes a human or humanized binding domain (e.g., scfv) including a variable heavy chain including a CDRH1 sequence including GYGMH (SEQ ID NO: 93), a CDRH2 sequence including VIWYDGSNKYYADSVKG (SEQ ID NO: 94), and a CDRH3 sequence including DTGDRFFDY (SEQ ID NO: 95). These reflect CDR sequences that bind CD64.

A number of antibodies specific for CD64 are known to those of skill in the art and can be readily characterized for sequence, epitope binding, and affinity. See, for example, U.S. Pat. No. 7,378,504, WO 2006/131953, and WO 2008/074867. Commercially available antibodies for CD64 can be obtained from Ancell, Bayport, MN; Thermo Fisher, Waltham, MA; Abcam, Cambridge, MA; LifeSpan Biosciences, Inc., Seattle, WA; and Novus Biologicals, Littleton, CO. In particular embodiments, anti-CD64 antibodies include: mouse monoclonal anti-CD64 antibody clone 32-2; mouse monoclonal anti-CD64 antibody clone UMAB74; rat monoclonal anti-CD64 antibody clone 290322; mouse monoclonal anti-CD64 antibody clone 10.1; and mouse monoclonal anti-CD64 antibody clone 1D3.

In particular embodiments, CD86 is targeted on M1 macrophages. A number of antibodies specific for CD86 are known to those of skill in the art and can be readily characterized for sequence, epitope binding, and affinity. See, for example, WO 2004/076488, U.S. Pat. No. 8,378,082 (mAb 2D4) and U.S. Pat. No. 6,346,248 (IG10H6D10). Commercially available antibodies for CD86 can be obtained from Thermo Fisher, Waltham, MA; Miltenyi Biotec, Bergisch Gladbach, Germany; LifeSpan Biosciences, Inc., Seattle, WA; Bio-Rad, Hercules, CA; and Novus Biologicals, Littleton, CO. In particular embodiments, anti-CD86 antibodies include: mouse monoclonal anti-CD86 antibody clone BU63; polyclonal goat anti-CD86 antibody recognizing a region including Ala23 to His244 of human CD86; mouse monoclonal anti-CD86 antibody clone IT2.2; rabbit monoclonal anti-CD86 antibody clone BFF-3; and mouse monoclonal anti-CD86 antibody clone C86/1146.

Other agents that can facilitate internalization by and/or transfection of lymphocytes, such as poly(ethyleneimine)/DNA (PEI/DNA) complexes can also be used.

(6) Compositions. The particles disclosed herein can be provided as part of compositions formulated for administration to subjects. Compositions include a particle disclosed herein and a pharmaceutically acceptable carrier.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol or mannitol.

Exemplary stabilizers include organic sugars, polyhydric sugar alcohols, polyethylene glycol, sulfur-containing reducing agents, amino acids, low molecular weight polypeptides, proteins, immunoglobulins, hydrophilic polymers or polysaccharides.

In particular embodiments, compositions are formulated for intraperitoneal, intravenous, or intracranial injection. The compositions disclosed herein can further be formulated for intraarterial, intranodal, intralymphatic, intratumoral, intramuscular, oral, and/or subcutaneous administration and more particularly by intraarterial, intranodal, intralymphatic, intratumoral, intramuscular, and/or subcutaneous injection. The compositions disclosed herein can be formulated for administration by infusion, perfusion, or ingestion.

For injection, compositions can be formulated as aqueous solutions, such as in buffers including Hanks' solution, Ringer's solution, or physiological saline. The aqueous solutions can contain formulatory agents such as suspending, stabilizing and/or dispersing agents. Alternatively, the formulation can be in lyophilized and/or powder form for constitution with a suitable vehicle, e.g., sterile pyrogen-free water, before use.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as sparingly soluble salts.

Additionally, compositions can be formulated as sustained-release systems utilizing semipermeable matrices of solid polymers containing particles. Various sustained-release materials have been established and are well known by those of ordinary skill in the art. Sustained-release systems may, depending on their chemical nature, release particles following administration for a few weeks up to over 100 days.

For oral administration, the compositions can be formulated as tablets, pills, dragees, capsules, liquids, gels, syrups, slurries, suspensions and the like.

When formulated to treat cancer, the disclosed compositions can also include nucleotides carrying one or more anticancer genes selected from p53, RB, BRCA1, E1A, bcl-2, MDR-1, p21, p16, bax, bcl-xs, E2F, IGF-I VEGF, angiostatin, oncostatin, endostatin, GM-CSF, IL-12, IL-2, IL-4, IL-7, IFN-γ, TNFα and/or HSV-tk.

Any composition formulation disclosed herein can advantageously include any other pharmaceutically acceptable carriers which include those that do not produce significantly adverse, allergic or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

In particular embodiments, the particles are provided as part of a composition that can include, for example, at least 0.1% w/v or w/w particles; at least 1% w/v or w/w particles; at least 10% w/v or w/w particles; at least 20% w/v or w/w particles; at least 30% w/v or w/w particles; at least 40% w/v or w/w particles; at least 50% w/v or w/w particles; at least 60% w/v or w/w particles; at least 70% w/v or w/w particles; at least 80% w/v or w/w particles; at least 90% w/v or w/w particles; at least 95% w/v or w/w particles; or at least 99% w/v or w/w particles.

Methods of use. Methods disclosed herein include altering the activation state of macrophages from an inactivated state to an activated state by introducing into macrophages nanoparticles including nucleotides encoding one or more IRFs and IKKβ. In particular embodiments, the altering results in reducing the percentage of macrophages in an inactivated state (e.g., M2 macrophages) in a population of macrophages treated with nanoparticles including nucleotides encoding one or more IRFs and IKKβ by 5-fold, 10-fold, 15-fold, 20-fold, or more compared to the percentage of macrophages in an inactivated state that have not been treated with the nanoparticles including nucleotides encoding one or more IRFs and IKKβ. In particular embodiments, the altering results in reducing the number of macrophages in an inactivated state (e.g., M2 macrophages) in a population of macrophages treated with the nanoparticles including nucleotides encoding one or more IRFs and IKKβ by 5-fold, 10-fold, 20-fold, 30-fold, 40-fold, 50-fold, 60-fold, 70-fold, 80-fold, 90-fold, 100-fold, or more compared to the number of macrophages in an inactivated state that have not been treated with the nanoparticles including nucleotides encoding one or more IRFs and IKKβ. In particular embodiments, the altering results in increasing the percentage of macrophages in an activated state (e.g., M1 macrophages) in a population of macrophages treated with the nanoparticles including nucleotides encoding one or more IRFs and IKKβ by 5-fold, 10-fold, 15-fold, 20-fold, or more compared to the percentage of macrophages in an activated state that have not been treated with the nanoparticles including nucleotides encoding one or more IRFs and IKKβ. In particular embodiments, the altering results in increasing the number of macrophages in an activated state in a population of macrophages treated with the nanoparticles including nucleotides encoding one or more IRFs and IKKβ by 5-fold, 10-fold, 20-fold, 30-fold, 40-fold, 50-fold, 60-fold, 70-fold, 80-fold, 90-fold, 100-fold, or more compared to the number of macrophages in an activated state that have not been treated with the nanoparticles including nucleotides encoding one or more IRFs and IKKβ.

In particular embodiments, altering the activation state of macrophages from an inactivated state to an activated state by introducing into macrophages nanoparticles including nucleotides encoding one or more IRFs and IKKβ results in: restoring lymphocyte migration and infiltration into solid tumors; increasing release of pro-inflammatory (anti-tumor) cytokines including IL-1B, IL-12, IFNγ, and/or TNFα by 1.5-fold, 2-fold, 2.5-fold, 3-fold, 3.5-fold, 4-fold, 5-fold, 6-fold, 7-fold, 8-fold, 9-fold, 10-fold, 15-fold, 20-fold, or more; reducing release of cytokines associated with M2 macrophage phenotype including IL-6 by 1.5-fold, 2-fold, 2.5-fold, 3-fold, 3.5-fold, 4-fold, 5-fold, 6-fold, 7-fold, 8-fold, 9-fold, 10-fold, 15-fold, 20-fold, or more.

In particular embodiments, altering the activation state of macrophages from an inactivated state to an activated state includes introducing into macrophages nanoparticles including nucleotides encoding IRF5 and IRF8. In particular embodiments, altering the activation of macrophages from an inactivated state to an activated state includes introducing into macrophages nanoparticles including nucleotides encoding mutant IRFs that are constitutively active or more active than their wild type counterpart IRFs.

Methods disclosed herein include treating subjects (humans, veterinary animals, livestock and research animals) with compositions disclosed herein. Treating subjects includes delivering a therapeutically effective amount. Therapeutically effective amounts can provide effective amounts, prophylactic treatments and/or therapeutic treatments.

An “effective amount” is the amount of a compound necessary to result in a desired physiological change in a subject. Effective amounts are often administered for research purposes. Effective amounts disclosed herein immunomodulate cells in a subject. In particular embodiments, the cells to be immunomodulated are immunosuppressed cells. In particular embodiments, the cells to be immunomodulated are macrophages. In particular embodiments, immunomodulation of macrophages includes switching immunosuppressed macrophages into activated macrophages. In particular embodiments, immunomodulation of macrophages includes switching M2 macrophages to M1 macrophages. In particular embodiments, cells to be immunomodulated include immunosuppressed cells including MDSC, Treg, DCreg, neutrophils, Th17, Breg, and/or MSC. In particular embodiments, immunomodulation of immunosuppressed cells includes phenotypic and/or functional switch of the immunosuppressed cells from being protumor to being antitumor.

A “prophylactic treatment” includes a treatment administered to a subject who does not display signs or symptoms of a disease or condition or displays only early signs or symptoms of the disease or condition such that treatment is administered for the purpose of diminishing, preventing, or decreasing the risk of developing the disease or condition further. Thus, a prophylactic treatment functions as a preventative treatment against a disease or disorder. In particular embodiments, a prophylactic treatment includes administration of the compositions disclosed herein to a subject who had cancer but is in remission such that treatment is administered for the purpose of reducing or delaying the occurrence of relapse.

A “therapeutic treatment” includes a treatment administered to a subject who displays symptoms or signs of a disease or condition and is administered to the subject for the purpose of diminishing or eliminating those signs or symptoms of the disease or condition. In particular embodiments, a therapeutic treatment includes administration of the compositions disclosed herein to a subject who has cancer to diminish or eliminate tumors and/or metastasis.

In particular embodiments, therapeutically effective amounts provide an anti-cancer effect in a subject. Cancer (medical term: malignant neoplasm) refers to a class of diseases in which a group of cells display uncontrolled growth (division beyond the normal limits), invasion (intrusion on and destruction of adjacent tissues), and sometimes metastasis. “Metastasis” refers to the spread of cancer cells from their original site of proliferation to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential.

In particular embodiments, therapeutically effective amounts provide an anti-tumor effect in a subject. A “tumor” is a swelling or lesion formed by an abnormal growth of cells (called neoplastic cells or tumor cells). A “tumor cell” is an abnormal cell that divides by a rapid, uncontrolled cellular proliferation and continues to divide after the stimuli that initiated the new division cease. Tumors show partial or complete lack of structural organization and functional coordination with the normal tissue, and usually form a distinct mass of tissue, which may be either benign, pre-malignant or malignant.

An anti-tumor effect refers to a biological effect, which can be manifested by a decrease in the number of tumor cells, a decrease in the number of metastases, a decrease in tumor volume, an increase life expectancy, induced apoptosis of cancer cells, induced cancer cell death, induced chemo- or radiosensitivity in cancer cells, inhibited angiogenesis near cancer cells, inhibited cancer cell proliferation, inhibited tumor growth, prevented metastasis, prolonged life for a subject, reduced cancer-associated pain, reduced number of metastases, and/or reduced relapse or re-occurrence of the cancer following treatment. Accordingly, the compositions disclosed herein can be used to treat a variety of cancers, can prevent or significantly delay metastasis, and/or can prevent or significantly delay relapse. In particular embodiments, overall survival of a subject with cancer treated with a nanoparticle composition disclosed herein is improved by 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, or more as compared to a control subject with the same cancer not treated with the nanoparticle composition. In particular embodiments, the number of metastases in a subject with cancer treated with a nanoparticle composition disclosed herein is decreased by 1.5-fold, 2-fold, 3-fold, 4-fold, 5-fold, 6-fold, 7-fold, 8-fold, 9-fold, 10-fold, or more as compared to a control subject with the same cancer not treated with the nanoparticle composition.

In particular embodiments, a therapeutic treatment includes administration of the compositions disclosed herein in combination with another therapy to a subject who has cancer to diminish or eliminate tumors. In particular embodiments, the therapy to use in combination with the compositions disclosed herein include cancer vaccines, CAR immunotherapy (e.g., CAR-T immunotherapy), chemotherapy, radiotherapy, hormone therapy, signal transduction inhibitors, gene expression modulators, apoptosis inducers, angiogenesis inhibitors, and monoclonal antibodies that deliver toxic molecules. In particular embodiments, administration of a nanoparticle composition disclosed herein in combination with radiotherapy to a subject who has cancer improves overall survival by 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, or more as compared to a control subject with the same cancer not administered the nanoparticle composition in combination with radiotherapy.

Cancers that can be treated with systems and methods disclosed herein include ovarian cancer, breast cancer, brain cancer, melanomas, lung metastases, seminomas, teratomas, neuroblastomas, gliomas, rectal cancer, endometrial cancer, kidney cancer, adrenal cancer, thyroid cancer, skin cancer, cervical cancer, intestinal cancer, liver cancer, colon cancer, stomach cancer, head and neck cancer, gastrointestinal cancer, lymph node cancer, esophagus cancer, colorectal cancer, pancreatic cancer, ear, nose and throat (ENT) cancer, prostate cancer, cancer of the uterus, lung cancer, and metastases thereof.

As indicated, the teachings of the current disclosure can also be used in methods of modulating the activation state of immune cells to inactivate the immune system in conditions such as autoimmune diseases. In particular embodiments, altering the activation state of macrophages from an activated state to an inactivated state in autoimmune diseases includes introducing into macrophages nanoparticles including nucleotides encoding IRFs that induce M2 phenotypes. Particular embodiments of IRFs that induce M2 phenotypes include IRF3 and/or IRF4. In particular embodiments, altering the activation state of macrophages from an activated state to an inactivated state in autoimmune diseases includes introducing into macrophages nanoparticles including nucleotides encoding GILZ (Glucocorticoid-induced leucine zipper) transcription factor which mediates glucocorticoid anti-inflammatory effects and can induce M2 phenotypes. In particular embodiments, altering the activation state of macrophages from an activated state to an inactivated state in autoimmune diseases includes introducing into macrophages nanoparticles including nucleotides encoding GILZ and IRF4. Exemplary autoimmune diseases include acute necrotizing hemorrhagic encephalopathy, allergic asthma, alopecia areata, anemia, aphthous ulcer, arthritis (including rheumatoid arthritis, juvenile rheumatoid arthritis, osteoarthritis, psoriatic arthritis), asthma, autoimmune thyroiditis, conjunctivitis, Crohn's disease, cutaneous lupus erythematosus, dermatitis (including atopic dermatitis and eczematous dermatitis), diabetes, diabetes mellitus, erythema nodosum leprosum, keratoconjunctivitis, multiple sclerosis, myasthenia gravis, psoriasis, scleroderma, Sjogren's syndrome, including keratoconjunctivitis sicca secondary to Sjogren's syndrome, Stevens-Johnson syndrome, systemic lupus erythematosus, ulcerative colitis, vaginitis and Wegener's granulomatosis.

For administration, therapeutically effective amounts (also referred to herein as doses) can be initially estimated based on results from in vitro assays and/or animal model studies. For example, a dose can be formulated in animal models to achieve a circulating concentration range that includes an IC₅₀ as determined in cell culture against a particular target. Such information can be used to more accurately determine useful doses in subjects of interest.

The actual dose amount administered to a particular subject can be determined by a physician, veterinarian or researcher taking into account parameters such as physical and physiological factors including target, body weight, severity of condition, type of disease, previous or concurrent therapeutic interventions, idiopathy of the subject and route of administration.

Useful doses often range from 0.1 to 5 μg/kg or from 0.5 to 1 μg/kg. In particular embodiments, a dose can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 to 5 mg/kg or from 0.5 to 1 mg/kg. In particular embodiments, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, 200 mg/kg, 250 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly. In particular embodiments, therapeutically effective amounts can be achieved by administering repeated doses during the course of a treatment regimen.

The nanoparticle compositions described herein can be administered by injection, inhalation, infusion, perfusion, lavage or ingestion. Routes of administration can include intravenous, intradermal, intraarterial, intraparenteral, intranasal, intranodal, intralymphatic, intraperitoneal, intracranial, intralesional, intraprostatic, intravaginal, intrarectal, topical, intrathecal, intratumoral, intramuscular, intravesicular, oral, subcutaneous, and/or sublingual administration and more particularly by intravenous, intratumoral, intraperitoneal, and/or intracranial injection. Local administration includes administration of a therapeutically effective amount of a composition disclosed herein to a particular region, organ, or cavity of the body. For example, intraperitoneal injection can be used to deliver a therapeutic to treat ovarian cancer or intracranial injection can be used to deliver a therapeutic to treat a glioma. Administration of a therapeutic at a tumor site can include ligand mediated targeting of a therapeutic (e.g., nanoparticle compositions) to tumor cells and/or tumor supporting cells and not to healthy tissue using targeting ligands as described above. Administration of a therapeutic at a tumor site can include passive targeting of a therapeutic (e.g., nanoparticle compositions) to tumor cells and/or tumor supporting cells and not to healthy tissue. Particular embodiments of passive targeting can include enhanced permeability and retention (EPR) phenomenon based on size range of nanoparticles and the leaky vasculature and impaired lymphatic drainage of tumor tissues. Systemic administration, by contrast, is body-wide, and is typically achieved by intravenous injection of a composition or therapeutic into the circulation. Systemic administration of a therapeutic can be useful for less localized forms of cancer, such as cancers that have metastasized.

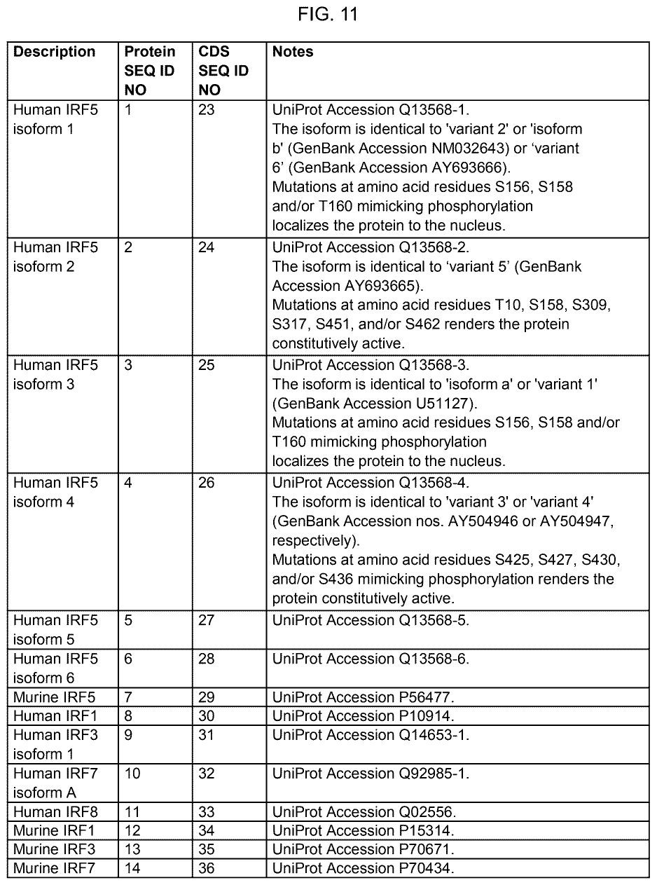

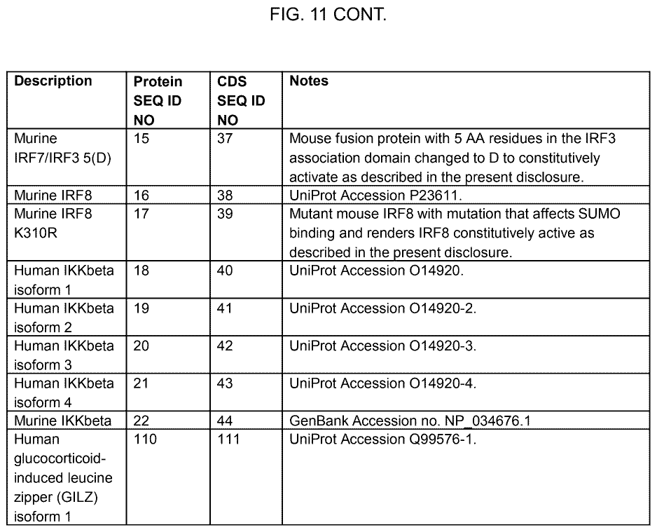

FIG. 11 provides exemplary sequences (SEQ ID NO: 1-44, 110, and 111) supporting the disclosure. CDR sequences are also described herein. The current disclosure includes variants of these sequences. Variants of protein sequences can include those having one or more conservative amino acid substitutions or one or more non-conservative substitutions that do not adversely affect the function of the protein. A “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gln); Group 4: Arginine (Arg), Lysine (Lys), Histidine (His); Group 5: Isoleucine (Ile), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W. H. Freeman and Company.

A fragment of a protein consists of less than the complete amino acid sequence of the corresponding protein, but retains the function of the full length protein.

Variants of nucleotide sequences can include one or more of degenerate codons, sequence polymorphisms, and mutations wherein such alterations do not affect the function of the encoded activation regulator or do not substantially affect the function of the encoded activation regulator.

In particular embodiments, variants of sequences include sequences with at least 70% sequence identity, at least 80%, at least 85%, at least 90%, at least 95%, at least 96%, at least 97%, at least 98%, or at least 99% sequence identity to the sequences described or disclosed herein.

“% sequence identity” refers to a relationship between two or more sequences, as determined by comparing the sequences. In the art, “identity” also means the degree of sequence relatedness between sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine % sequence identity are designed to give the best match between the sequences tested. Methods to determine % sequence identity and similarity are codified in publicly available computer programs. Sequence alignments and % sequence identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, NY. Within the context of this disclosure it will be understood that where sequence analysis software is used for analysis, the results of the analysis are based on the “default values” of the program referenced. “Default values” will mean any set of values or parameters, which originally load with the software when first initialized.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A method of altering an activation state of immune cells in vivo including:
administering nanoparticles including nucleotides encoding one or more interferon regulatory factors (IRFs) thereby altering the activation state of immune cells in vivo.
2. A method of embodiment 1 wherein the immune cell is a macrophage, a regulatory T cell (TREG), a myeloid-derived suppressor cell (MDSC), a regulatory dendritic cell (DCreg), a neutrophil, a T helper 17 cell (Th17), a regulatory B cell (Breg), and/or a mesenchymal stromal cell (MSC).
3. A method of embodiment 1 or 2 wherein the nanoparticles include a positively-charged core, a poly(β)-amino ester core, star-shaped polymers, a polyglutamic acid coating, a hyaluronic acid coating, a neutrally-charged coating, and/or liposomal nanoparticles.
4. A method of any one of embodiments 1-3 wherein the nanoparticles are <130 nm.
5. A method of any one of embodiments 1˜4 wherein the nucleotides include in vitro transcribed mRNA.
6. A method of any one of embodiments 1-5 wherein the nucleotides are encapsulated within a core.
7. A method of any one of embodiments 1-6 wherein the encoded one or more IRFs lack a functional autoinhibitory domain.
8. A method of any one of embodiments 1-7 wherein the encoded one or more IRFs lack a functional nuclear export signal (NES).
9. A method of any one of embodiments 1-8 wherein the administering is locally administering.
10. A method of embodiment 9 wherein the locally administering is intraperitoneal or intracranial.
11. A method of any one of embodiments 1-9 wherein the administering is systemic administering.
12. A method of any one of embodiments 1-11 wherein the nanoparticles further include a targeting ligand.
13. A method of embodiment 12 wherein the targeting ligand is linked to a coating.
14. A method of any one of embodiments 1-13 wherein the activation state is altered from an inactivated state to an activated state.
15. A method of any one of embodiments 1-14 wherein the immune cells include macrophages.
16. A method of embodiment 15 wherein the macrophages are within a tumor.
17. A method of embodiment 16 wherein the tumor is an ovarian cancer tumor, a glioblastoma tumor, or a metastatic lung cancer tumor.
18. A method of any one of embodiments 1-17 wherein the encoded one or more IRFs is selected from IRF1, IRF3, IRF5, IRF7, IRF8, and/or a fusion of IRF7 and IRF3.
19. A method of any one of embodiments 1-18 wherein the encoded one or more IRFs is selected from a sequence having >90%, >95%, or greater than 98% identity to SEQ ID NOs: 1-17.
20. A method of any one of embodiments 1-19 wherein the encoded one or more IRFs is IRF5 selected from SEQ ID NOs: 1-7.
21. A method of embodiment 20 wherein IRF5 is SEQ ID NO: 1.
22. A method of embodiment 20 or 21 wherein IRF5 is SEQ ID NO: 1 or SEQ ID NO: 3 with one or more mutations selected from S156D, S158D and T160D.
23. A method of any one of embodiments 20-22 wherein IRF5 is SEQ ID NO: 2 with one or more mutations selected from T10D, S158D, S309D, S317D, S451D, and S462D.
24. A method of any one of embodiments 20-23 wherein IRF5 is SEQ ID NO: 4 with one or more mutations selected from S425D, S427D, S430D, and S436D.
25. A method of any one of embodiments 1-24 wherein the encoded one or more IRFs is IRF1 selected from SEQ ID NOs: 8 and 12.
26. A method of any one of embodiments 1-24 wherein the encoded one or more IRFs is IRF8 selected from SEQ ID NOs: 11, 16, and 17.
27. A method of embodiment 26 wherein IRF8 is SEQ ID NO: 11 with a K310R mutation.
28. A method of any one of embodiments 1-27 wherein the encoded one or more IRFs includes an IRF7/IRF3 fusion protein including an N-terminal IRF7 DNA binding domain (DBD) and constitutively active domain (CAD) and C-terminal IRF3 NES (Nuclear Export Signal) and association domains.
29. A method of embodiment 28 wherein the IRF7/IRF3 fusion protein further includes mutations mimicking phosphorylation in the IRF3 association domain.
30. A method of embodiment 28 or 29 wherein the IRF7/IRF3 fusion protein is set forth in SEQ ID NO: 15.
31. A method of any one of embodiments 1-30 wherein the nanoparticles further include nucleotides encoding IKKβ.
32. A method of embodiment 31 wherein the encoded IKKβ is selected from a sequence having >90%, >95%, or greater than 98% identity to SEQ ID NOs: 18-22.
33. A method of embodiment 31 or 32 wherein the encoded IKKβ is selected from SEQ ID NOs: 18-22.
34. A method of any one of embodiments 1-33 wherein the nucleotides include a sequence selected from SEQ ID NOs: 23-44.
35. A method of any one of embodiments 12-34 wherein the targeting ligand binds CD206, CD163, or CD23.
36. A method of any one of embodiments 12-35 wherein the targeting ligand is di-mannose.
37. A method of any one of embodiments 31-36 wherein the nucleotides encoding one or more IRFs and IKKβ are encapsulated in the same nanoparticle.
38. A method of any one of embodiments 31-37 wherein the nucleotides encoding one or more IRFs and IKKβ are encapsulated in different nanoparticles.
39. A method of any one of embodiments 1-38 wherein altering the activation state of immune cells includes reducing the percentage of immune cells in the inactivated state in a population of immune cells by 5-fold, 10-fold, 15-fold, 20-fold, or more.
40. A method of any one of embodiments 1-39 wherein altering the activation state of immune cells includes reducing the number of immune cells in the inactivated state in a population of immune cells by 5-fold, 10-fold, 20-fold, 30-fold, 40-fold, 50-fold, 60-fold, 70-fold, 80-fold, 90-fold, 100-fold, or more.
41. A method of any one of embodiments 1-40 wherein altering the activation state of immune cells includes increasing the percentage of immune cells in the activated state in a population of immune cells by 5-fold, 10-fold, 15-fold, 20-fold, or more.
42. A method of any one of embodiments 1-41 wherein altering the activation state of immune cells includes increasing the number of immune cells in the activated state in a population of immune cells by 5-fold, 10-fold, 20-fold, 30-fold, 40-fold, 50-fold, 60-fold, 70-fold, 80-fold, 90-fold, 100-fold, or more.
43. A method of any one of embodiments 1-13 wherein the activation state is altered from an activated state to an inactivated state.
44. A method of any one of embodiments 1-13, and 43 wherein the immune cells include macrophages.
45. A method of any one of embodiments 1-13, 43, and 44 wherein the encoded one or more IRFs is IRF4.
46. A method of any one of embodiments 1-13, and 43-45 wherein the nanoparticles further include nucleotides encoding glucocorticoid-induced leuzine zipper (GILZ).
47. A method of any one of embodiments 12, and 43-46 wherein the targeting ligand binds CD38, G-protein coupled receptor 18 (Gpr18), formyl peptide receptor 2 (Fpr2), CD64, or CD68.
48. A method of any one of embodiments 1-13, and 43-47 wherein altering the activation state of immune cells includes reducing the percentage of immune cells in the activated state in a population of immune cells by 5-fold, 10-fold, 15-fold, 20-fold, or more.
49. A method of any one of embodiments 1-13, and 43-48 wherein altering the activation state of immune cells includes reducing the number of immune cells in the activated state in a population of immune cells by 5-fold, 10-fold, 20-fold, 30-fold, 40-fold, 50-fold, 60-fold, 70-fold, 80-fold, 90-fold, 100-fold, or more.
50. A method of any one of embodiments 1-13, and 43-49 wherein altering the activation state of immune cells includes increasing the percentage of immune cells in the inactivated state in a population of immune cells by 5-fold, 10-fold, 15-fold, 20-fold, or more.
51. A method of any one of embodiments 1-13, and 43-50 wherein altering the activation state of immune cells includes increasing the number of immune cells in the inactivated state in a population of immune cells by 5-fold, 10-fold, 20-fold, 30-fold, 40-fold, 50-fold, 60-fold, 70-fold, 80-fold, 90-fold, 100-fold, or more.
52. A method of treating cancer in a subject in need thereof including altering the activation state of tumor-associated macrophages in a tumor within the subject from inactivated to activated thereby treating cancer in the subject in need thereof.
53. A method of embodiment 52 wherein the tumor is an ovarian cancer tumor, a glioblastoma tumor, or a metastatic lung cancer tumor.
54. A method of embodiment 52 or 53 wherein the altering follows administration of a therapeutically effective amount of nanoparticles including nucleotides that encode one or more transcription factors that alter the activation state of tumor-associated macrophages from inactivated to activated.
55. A method of embodiment 54 wherein the nanoparticles include a positively-charged core, a poly(β)-amino ester core, star-shaped polymers, a polyglutamic acid coating, a hyaluronic acid coating, a neutrally-charged coating, and/or liposomal nanoparticles.
56. A method of embodiment 54 or 55 wherein the nanoparticles are <130 nm.
57. A method of any one of embodiments 54-56 wherein the nucleotides include in vitro transcribed mRNA.
58. A method of any one of embodiments 54-57 wherein the nucleotides are encapsulated within a core.
59. A method of any one of embodiments 54-58 wherein the administration is local administration.
60. A method of embodiment 59 wherein the local administration is intraperitoneal or intracranial.
61. A method of any one of embodiments 54-59 wherein the administration is systemic administration.
62. A method of any one of embodiments 54-61 wherein the encoded one or more transcription factors include one or more interferon regulatory factors (IRFs).
63. A method of embodiment 62 wherein the encoded one or more IRFs lack a functional autoinhibitory domain.
64. A method of embodiment 62 or 63 wherein the encoded one or more IRFs lack a functional nuclear export signal (NES).
65. A method of any one of embodiments 62-64 wherein the encoded one or more IRFs is selected from IRF1, IRF3, IRF5, IRF7, IRF8, and/or a fusion of IRF7 and IRF3.
66. A method of any one of embodiments 62-65 wherein the encoded one or more IRFs is selected from a sequence having >90%, >95%, or greater than 98% identity to SEQ ID NOs: 1-17.
67. A method of any one of embodiments 62-66 wherein the encoded one or more IRFs is IRF5 selected from SEQ ID NOs: 1-7.
68. A method of embodiment 67 wherein IRF5 is SEQ ID NO: 1.
69. A method of embodiment 67 or 68 wherein IRF5 is SEQ ID NO: 1 or SEQ ID NO: 3 with one or more mutations selected from S156D, S158D and T160D.
70. A method of any one of embodiments 67-69 wherein IRF5 is SEQ ID NO: 2 with one or more mutations selected from T10D, S158D, S309D, S317D, S451D, and S462D.
71. A method of any one of embodiments 67-70 wherein IRF5 is SEQ ID NO: 4 with one or more mutations selected from S425D, S427D, S430D, and S436D.
72. A method of any one of embodiments 62-71 wherein the encoded one or more IRFs is IRF1 selected from SEQ ID NOs: 8 and 12.
73. A method of any one of embodiments 62-72 wherein the encoded one or more IRFs is IRF8 selected from SEQ ID NOs: 11, 16, and 17.
74. A method of embodiment 73 wherein IRF8 is SEQ ID NO: 11 with a K310R mutation.
75. A method of any one of embodiments 62-74 wherein the encoded one or more IRFs includes an IRF7/IRF3 fusion protein including an N-terminal IRF7 DNA binding domain (DBD) and constitutively active domain (CAD) and C-terminal IRF3 NES (Nuclear Export Signal) and association domains.
76. A method of embodiment 75 wherein the IRF7/IRF3 fusion protein further includes mutations mimicking phosphorylation in the IRF3 association domain.
77. A method of embodiment 75 or 76 wherein the IRF7/IRF3 fusion protein is set forth in SEQ ID NO: 15.
78. A method of any one of embodiments 54-77 wherein the nanoparticles further include nucleotides encoding IKKβ.
79. A method of embodiment 78 wherein the encoded IKKβ is selected from a sequence having >90%, >95%, or greater than 98% identity to SEQ ID NOs: 18-22.
80. A method of embodiment 78 or 79 wherein the encoded IKKβ is selected from SEQ ID NOs: 18-22.
81. A method of any one of embodiments 54-79 wherein the nucleotides include a sequence selected from SEQ ID NOs: 23-44.
82. A method of any one of embodiments 54-81 wherein the nanoparticles further include a targeting ligand.
83. A method of embodiment 82 wherein the targeting ligand is linked to a coating.
84. A method of embodiment 82 or 83 wherein the targeting ligand binds CD206, CD163, or CD23.
85. A method of any one of embodiments 82-84 wherein the targeting ligand is di-mannose.
86. A method of any one of embodiments 54-85 wherein the nucleotides encoding one or more IRFs and IKKβ are encapsulated in the same nanoparticle.
87. A method of any one of embodiments 54-86 wherein the nucleotides encoding one or more IRFs and IKKβ are encapsulated in different nanoparticles.
88. A method of any one of embodiments 54-87 wherein altering the activation state of macrophages includes reducing the percentage of macrophages in the inactivated state in a population of macrophages within the tumor by 5-fold, 10-fold, 15-fold, 20-fold, or more.
89. A method of any one of embodiments 54-88 wherein altering the activation state of macrophages includes reducing the number of macrophages in the inactivated state in a population of macrophages within the tumor by 5-fold, 10-fold, 20-fold, 30-fold, 40-fold, 50-fold, 60-fold, 70-fold, 80-fold, 90-fold, 100-fold, or more.
90. A method of any one of embodiments 54-89 wherein altering the activation state of macrophages includes increasing the percentage of macrophages in the activated state in a population of macrophages within the tumor by 5-fold, 10-fold, 15-fold, 20-fold, or more.
91. A method of any one of embodiments 54-90 wherein altering the activation state of macrophages includes increasing the number of macrophages in the activated state in a population of macrophages within the tumor by 5-fold, 10-fold, 20-fold, 30-fold, 40-fold, 50-fold, 60-fold, 70-fold, 80-fold, 90-fold, 100-fold, or more.
92. A method of any one of embodiments 54-91 further including administering in combination with the therapeutically effective amount of nanoparticles a therapy selected from cancer vaccines, chimeric antigen receptor (CAR) immunotherapy, chemotherapy, radiotherapy, hormone therapy, signal transduction inhibitors, gene expression modulators, apoptosis inducers, angiogenesis inhibitors, and monoclonal antibodies that deliver toxic molecules.
93. A method of treating an autoimmune disease in a subject in need thereof including altering the activation state of macrophages within the subject from activated to inactivated thereby treating an autoimmune disease in the subject in need thereof.
94. A method of embodiment 93 wherein the autoimmune disease includes acute necrotizing hemorrhagic encephalopathy, allergic asthma, alopecia areata, anemia, aphthous ulcer, arthritis (including rheumatoid arthritis, juvenile rheumatoid arthritis, osteoarthritis, psoriatic arthritis), asthma, autoimmune thyroiditis, conjunctivitis, Crohn's disease, cutaneous lupus erythematosus, dermatitis (including atopic dermatitis and eczematous dermatitis), diabetes, diabetes mellitus, erythema nodosum leprosum, keratoconjunctivitis, multiple sclerosis, myasthenia gravis, psoriasis, scleroderma, Sjogren's syndrome, including keratoconjunctivitis sicca secondary to Sjogren's syndrome, Stevens-Johnson syndrome, systemic lupus erythematosus, ulcerative colitis, vaginitis and Wegener's granulomatosis.
95. A method of embodiment 93 or 94 wherein the altering follows administration of a therapeutically effective amount of nanoparticles including nucleotides encoding one or more transcription factors that alter the activation state of macrophages from activated to inactivated.
96. A method of embodiment 94 wherein the nanoparticles include a positively-charged core, a poly(β)-amino ester core, star-shaped polymers, a polyglutamic acid coating, a hyaluronic acid coating, a neutrally-charged coating, and/or liposomal nanoparticles.
97. A method of embodiment 94 or 95 wherein the nanoparticles are <130 nm.
98. A method of any one of embodiments 95-97 wherein the nucleotides include in vitro transcribed mRNA.
99. A method of any one of embodiments 95-98 wherein the nucleotides are encapsulated within a core.
100. A method of any one of embodiments 95-99 wherein the administration is local administration.
101. A method of embodiment 100 wherein the local administration is intraperitoneal or intracranial.
102. A method of any one of embodiments 92-99 wherein the administration is systemic administration.
103. A method of any one of embodiments 95-102 wherein the encoded one or more transcription factors include one or more interferon regulatory factors (IRFs).
104. A method of embodiment 103 wherein the encoded one or more IRFs lack a functional autoinhibitory domain.
105. A method of embodiment 103 or 104 wherein the encoded one or more IRFs lack a functional nuclear export signal (NES).
106. A method of any one of embodiments 103-105 wherein the encoded one or more IRFs is IRF4.
107. A method of any one of embodiments 95-106 wherein the nanoparticles further include nucleotides encoding glucocorticoid-induced leuzine zipper (GILZ).
108. A method of any one of embodiments 85-107 wherein the nanoparticles further include a targeting ligand.
109. A method of embodiment 108 wherein the targeting ligand is linked to a coating.
110. A method of embodiment 108 or 109 wherein the targeting ligand binds CD38, G-protein coupled receptor 18 (Gpr18), formyl peptide receptor 2 (Fpr2), CD64, or CD68.
111. A method of any one of embodiments 93-110 wherein altering the activation state of macrophages includes reducing the percentage of macrophages in the activated state in a population of macrophages by 5-fold, 10-fold, 15-fold, 20-fold, or more.
112. A method of any one of embodiments 93-111 wherein altering the activation state of macrophages includes reducing the number of macrophages in the activated state in a population of macrophages by 5-fold, 10-fold, 20-fold, 30-fold, 40-fold, 50-fold, 60-fold, 70-fold, 80-fold, 90-fold, 100-fold, or more.
113. A method of any one of embodiments 95-112 wherein altering the activation state of macrophages includes increasing the percentage of macrophages in the inactivated state in a population of macrophages by 5-fold, 10-fold, 15-fold, 20-fold, or more.
114. A method of any one of embodiments 95-113 wherein altering the activation state of macrophages includes increasing the number of macrophages in the inactivated state in a population of macrophages by 5-fold, 10-fold, 20-fold, 30-fold, 40-fold, 50-fold, 60-fold, 70-fold, 80-fold, 90-fold, 100-fold, or more.
115. A composition including nanoparticles including nucleotides encoding one or more interferon regulatory factors (IRFs).
116. A composition of embodiment 115 further including a pharmaceutically acceptable carrier.
117. A composition of embodiment 115 or 116 wherein the nanoparticles include a positively-charged core, a poly(β)-amino ester core, star-shaped polymers, a polyglutamic acid coating, a hyaluronic acid coating, a neutrally-charged coating, and/or liposomal nanoparticles.
118. A composition of any one of embodiments 115-117 wherein the nanoparticles are <130 nm.
119. A composition of any one of embodiments 115-118 wherein the nucleotides include in vitro transcribed mRNA.
120. A composition of any one of embodiments 115-119 wherein the nucleotides are encapsulated within a core.
121. A composition of any one of embodiments 115-120 wherein the encoded one or more IRFs lack a functional autoinhibitory domain.
122. A composition of any one of embodiments 115-121 wherein the encoded one or more IRFs lack a functional nuclear export signal (NES).
123. A composition of any one of embodiments 115-122 wherein the encoded one or more IRFs is selected from IRF1, IRF3, IRF5, IRF7, IRF8, and/or a fusion of IRF7 and IRF3.
124. A composition of any one of embodiments 115-123 wherein the encoded one or more IRFs is selected from a sequence having >90%, >95%, or greater than 98% identity to SEQ ID NOs: 1-17.
125. A composition of any one of embodiments 115-124 wherein the encoded one or more IRFs is IRF5 selected from SEQ ID NOs: 1-7.
126. A composition of embodiment 125 wherein IRF5 is SEQ ID NO: 1.
127. A composition of embodiment 125 or 126 wherein IRF5 is SEQ ID NO: 1 or SEQ ID NO: 3 with one or more mutations selected from S156D, S158D and T160D.
128. A composition of any one of embodiments 125-127 wherein IRF5 is SEQ ID NO: 2 with one or more mutations selected from T10D, S158D, S309D, S317D, S451D, and S462D.
129. A composition of any one of embodiments 125-128 wherein IRF5 is SEQ ID NO: 4 with one or more mutations selected from S425D, S427D, S430D, and S436D.
130. A composition of any one of embodiments 115-129 wherein the encoded one or more IRFs is IRF1 selected from SEQ ID NOs: 8 and 12.
131. A composition of any one of embodiments 115-130 wherein the encoded one or more IRFs is IRF8 selected from SEQ ID NOs: 11, 16, and 17.
132. A composition of embodiment 131 wherein IRF8 is SEQ ID NO: 11 with a K310R mutation.
133. A composition of any one of embodiments 115-132 wherein the encoded one or more IRFs includes an IRF7/IRF3 fusion protein including an N-terminal IRF7 DNA binding domain (DBD) and constitutively active domain (CAD) and C-terminal IRF3 NES (Nuclear Export Signal) and association domains.
134. A composition of embodiment 133 wherein the IRF7/IRF3 fusion protein further includes mutations mimicking phosphorylation in the IRF3 association domain.
135. A composition of embodiment 133 or 134 wherein the IRF7/IRF3 fusion protein is set forth in SEQ ID NO: 15.
136. A composition of any one of embodiments 115-135 wherein the nanoparticles further include nucleotides encoding IKKβ.
137. A composition of embodiment 136 wherein the encoded IKKβ is selected from a sequence having >90%, >95%, or greater than 98% identity to SEQ ID NOs: 18-22.
138. A composition of embodiment 136 or 137 wherein the encoded IKKβ is selected from SEQ ID NOs: 18-22.
139. A composition of any one of embodiments 115-138 wherein the nucleotides include a sequence selected from SEQ ID NOs: 23-44.
140. A composition of any one of embodiments 115-139 wherein the nanoparticles further include nucleotides carrying one or more anticancer genes selected from p53, RB, BRCA1, E1A, bcl-2, MDR-1, p21, p16, bax, bcl-xs, E2F, IGF-I VEGF, angiostatin, oncostatin, endostatin, GM-CSF, IL-12, IL-2, IL-4, IL-7, IFN-γ, TNFα and/or HSV-tk.
141. A composition of any one of embodiments 115-122 wherein the encoded one or more IRFs is IRF4.
142. A composition of any one of embodiments 115-122, and 141 wherein the nanoparticles further include nucleotides encoding glucocorticoid-induced leuzine zipper (GILZ).
143. A composition of any one of embodiments 115-142 wherein the nanoparticles further include a targeting ligand.
144. A composition of embodiment 143 wherein the targeting ligand is linked to a coating.
145. A composition of embodiment 143 or 144 wherein the targeting ligand binds CD206, CD163, or CD23.
146. A composition of embodiment 145 wherein the targeting ligand is di-mannose.
147. A composition of embodiment 143 or 144 wherein the targeting ligand binds CD38, G-protein coupled receptor 18 (Gpr18), formyl peptide receptor 2 (Fpr2), CD64, or CD68.
148. A composition of any one of embodiments 115-147 wherein the nucleotides encoding one or more IRFs, IKKβ, and/or GILZ are encapsulated in the same nanoparticle.
149. A composition of any one of embodiments 115-148 wherein the nucleotides encoding one or more IRFs, IKKβ, and/or GILZ are encapsulated in different nanoparticles.

Example 1. Materials and Methods. PbAE synthesis. The methods used to synthesize the polymer were described previously (Mangraviti A et al. (2015) ACS Nano 9:1236-1249). 1,4-butanediol diacrylate was combined with 4-amino-1-butanol in a 1:1 molar ratio of diacrylate to amine monomers. Acrylate-terminated poly(4-amino-1-butanol-co-1,4-butanediol diacrylate) was formed by heating the mixture to 90° C. with stirring for 24 hours. 2.3 g of this polymer was dissolved in 2 mL tetrahydrofuran (THF). To form the piperazine-capped 447 polymer, 786 mg of 1-(3-aminopropyl)-4-methylpiperazine in 13 mL THF was added to the polymer/THF solution and stirred at room temperature (RT) for 2 hours. The capped polymer was precipitated with 5 volumes of diethyl ether, washed with 2 volumes of fresh ether, and dried under vacuum for 1 day. Neat polymer was dissolved in dimethyl sulfoxide (DMSO) to a concentration of 100 mg/ml and stored at −20° C.

PGA conjugation to Di-mannose. α-D-mannopyranosyl-(1-→2)-α-D-mannopyranose (Di-mannose, Omicron Biochemicals Inc.) was modified into glycosylamine before being conjugated to polyglutamic acid (PGA). First, the Di-mannose (157 mg) was dissolved in 10.5 mL of saturated aqueous ammonium carbonate, then stirred at RT for 24 hours. On the second day, more solid ammonium carbonate was added until the Di-mannose precipitated from the reaction solution. The mixture was stirred until completion, as measured by TLC, followed by lyophilization to remove the excess ammonium carbonate. Complete removal of volatile salt was accomplished by re-dissolving the solid in methanol. These procedures created an amine on the anomeric carbon for future conjugation with PGA.

To conjugate aminated Di-mannose to PGA, the substrate was dissolved in water to 30 mg m/L, then sonicated for 10 minutes. Ethyl-N′-(3-dimethylaminopropyl) carbodiimide·HCl in water (4 mg/mL, 30 equiv.) was added with mixing at RT for 4 min. N-hydroxysulfosuccinimide in water (30 mg/mL, 35 equiv.) was incubated with the PGA/EDC solution for 1 minute. Aminated Dimannose in phosphate-buffered saline (PBS) was combined with the resulting activated PGA in a 44:1 molar ratio and mixed at RT for 6 h. Excess reagents were removed by dialysis against water for 24 hours.

mRNA synthesis. Codon-optimized mRNA for eGFP, IRF5, and IKK (TriLink Biotechnologies) were capped with the Anti-Reverse Cap Analog 3′-O-Me-m7G(5′)ppp(5′) G (ARCA), and fully substituted with the modified ribonucleotides pseudouridine (Ψ) and 5-methylcytidine (m5C).

Nanoparticle preparation. IRF5 and IKKβ mRNAs were combined at a 3:1 (w: w) ratio and diluted to 100 μg/mL in 25 mM sodium acetate (NaOAc) buffer (pH=5.2). Poly(β-amino esters)-447 (PbAE-447) polymer in DMSO (prepared as described above) was diluted from 100 μg/μL to 6 μg/μL, also in NaOAc buffer. To form the nanoparticles, PbAE-447 polymers were added to the mRNA at a ratio of 60:1 (w: w) and vortexed immediately for 15 seconds at a medium speed, then the mixture was incubated at RT for 5 min to allow the formation of PbAE-mRNA polyplexes. In the next step, 100 μg/mL PGA/Di-mannose in NaOAc buffer was added to the polyplexes solution, vortexed for 15 seconds at medium speed, and incubated for 5 min at room temperature. In this process, PGA/Di-mannose coated the surfaces of PbAE-mRNA polyplexes to form the final NPs. For long-term storage, D-sucrose (60 mg/mL) was added to the NP solutions as a cryoprotectant. The nanoparticles were snap-frozen in dry ice, then lyophilized. The dried NPs were stored at −20° C. or −80° C. until use. For in vivo experiments, lyophilized NPs were re-suspended in water at a 1:20 (w: v) ratio.

Characterization of nanoparticle size distribution and ζ-potential. The physiochemical properties of NPs (including hydrodynamic radius, polydispersity, Z-potential, and stability) were characterized using a Zetapals instrument (Brookhaven Instrument Corporation) at 25° C. To measure the hydrodynamic radius and polydispersity based on dynamic light scattering, NPs were diluted 5-fold into 25 mM NaOAc (pH=5.2). To measure the Z-potential, NPs were diluted 10-fold in 10 mM PBS (pH=7.0). To assess the stability of NPs, freshly prepared particles were diluted in 10 mM PBS buffer (pH=7.4). The hydrodynamic radius and polydispersity of NPs were measured every 10 minutes for 5 hours, and their sizes and particle concentrations were derived from Particle Tracking Analysis using a Nanosite 300 instrument (Malvern). To characterize the NPs using transmission electron microscopy, previously described protocols were followed (Smith T T et al. (2017) Nat Nanotechnol 12:813-820). Freshly made NPs (25 μL containing 0.83 μg of mRNA) were deposited on glow discharge-treated 200 mesh carbon/Formvar-coated copper grids. After 30 seconds, the grids were treated sequentially with 50% Karnovsky's fixative, 0.1 M cacodylate buffer, dH2O, then 1% (w/v) uranyl acetate. Samples were imaged with a JEOL JEM-1400 transmission electron microscope operating at 120 kV (JEOL USA).

Bone marrow derived macrophages (BMDMs) and other cell lines. To prepare BMDMs, bone marrow progenitor cells were harvested from mouse femurs following established protocols (Zhang X et al. (2008) Curr Protoc Immunol Chapter 14: Unit 14 11). These cells were cultured in complete medium [DMEM supplemented with 4.5 g/L D-glucose, L-glutamine, 10% heat-inactivated fetal bovine serum (FBS), 100 U/mL penicillin and 100 μg/mL, Glutamax 50 mL/500 mL, supplemented with 20 ng/ml M-CSF (Peprotech, cat #315-02)] at a seeding density of 0.5-1.0 e6/ml. Cells were allowed to differentiate into BMDMs ex vivo for 7 days under 5% CO2 at 37° C. Next, they were conditioned with macrophage-conditioned medium [macrophage complete medium supplemented with 20 ng/ml MPLA (Sigma, cat #L6895) or 20 ng/ml IL4 (eBioscience, cat #34-8041)]. BMDMs were used between 7-21 days ex vivo. The murine ovarian cancer cell line ID8, a gift from Dr. Katherine Roby (University of Kansas Medical Center, Kansas City, KS), was cultured in DMEM supplemented with 10% FBS, 100 U/mL penicillin, 5 μg/mL insulin, 5 g/mL transferrin, and 5 ng/ml sodium selenite (all Sigma-Aldrich). To generate the more aggressive vascular endothelial growth factor (VEGF)-expressing ID8 strain, ID8 tumor cells were transfected with the pUNO1 plasmid (Invivogen) encoding murine VEGF along with the blasticidin-resistance gene. To obtain stable transfectants, tumor cells were cultured in complete medium containing 10 μg/mL blasticidin (Invivogen) for 3 weeks. The B16F10 melanoma cell line (American Type Culture Collection) was cultured in complete RPMI 1640 medium with 10% FBS, 100 U/mL penicillin, 2 mM/L-glutamine, 1.5 g/L sodium bicarbonate, 4.5 g/L glucose, 10 mM HEPES, 1.0 mM sodium pyruvate, and 0.05 mM 2-mercaptoethanol. For in vivo bioluminescent imaging, both ID8-VEGF and B16F10 cell lines were retrovirally transduced with firefly luciferase. The DF-1 cell line carrying RACS-PDGFβ or RCAS-cre retrovirus was cultured in complete medium supplemented with 10% FBS and 100 U/mL penicillin under 5% CO2 at 39° C.

mRNA transfection of BMDMs. One day prior to transfection, BMDMs were reseeded on 24-well plates in macrophage complete medium at a concentration of 250,000/well. Before transfection, the complete medium was replaced with 300 μL unsupplemented DMEM. To transfect these cells, NPs containing 2 μg mRNA were added into the base medium and co-cultured with the BMDMs at 37° C. After 1 hour, medium containing NPs was removed, and the cells were cultured an additional 24 hours before evaluation of transfection efficiency and cell viability.

Transfection of BMDMs for macrophage signature gene analysis. BMDMs were reseeded on 24-well plates in conditioned medium 24 hours prior to transfection, allowing transformation of the cells into their phenotypes. M2-like macrophages were then exposed to either IRF5/IKKβ NPs carrying 25% eGFP mRNA as a reporter, or eGFP NPs (control) containing 2 μg mRNA, following the transfection protocol described above. After 24 hours, the top 10% percent of highly transfected BMDMs (as measured by eGFP expression) were sorted at 24 hours after transfection and were re-challenged in low-dose (10 ng/ml) IL4 medium for another 48 hours before RNA isolation. RNAs extracted from these cells were compared to those from standard M1- or M2-like macrophages so that signature genes associated with IRF5-NP treatment could be identified.

RNA isolation and preparation. To harvest RNAs, BMDMs were lysed in Trizol reagent (Ambion), and total RNAs were extracted and purified using RNeasy® Plus Universal Mini-Kits (QIAGEN) following the manufacturer's instructions. Sample RNA was quantified using a NanoDrop Microvolume Spectrophotometer (Thermo Fisher) and then subjected to quality control performed by the FHCRC Genomics Shared Resource with an Agilent 4200 TapeStation analyzer (Agilent).

Macrophage signature gene analysis by NanoString Technology. Gene expression values from stimulated BMDM cultures were measured using the nCounter® Myeloid Innate Immunity Panel (NanoString Technologies, Seattle, WA), which analyzes 770 genes occurring in 19 different pathways and processes them across 7 different myeloid cell types. The samples were tested using an nCounter Analysis System (NanoString Technologies, Seattle, WA). Raw data were processed and checked for quality using the R/Bioconductor NanoStringQCPro software package (Nickles D, Sandmann T, Ziman R and Bourgon R (2018) NanoStringQCPro: Quality metrics and data processing methods for NanoString mRNA gene expression data. R package version 1.10.0). Expression values were normalized to the geometric mean of housekeeping genes and log 2-transformed using nSolver 4.0 software (NanoString Technologies, Seattle, WA). False Discovery Rates for ratio data were calculated from the p-values returned by the t-tests using the Benjamini-Yekutieli method.

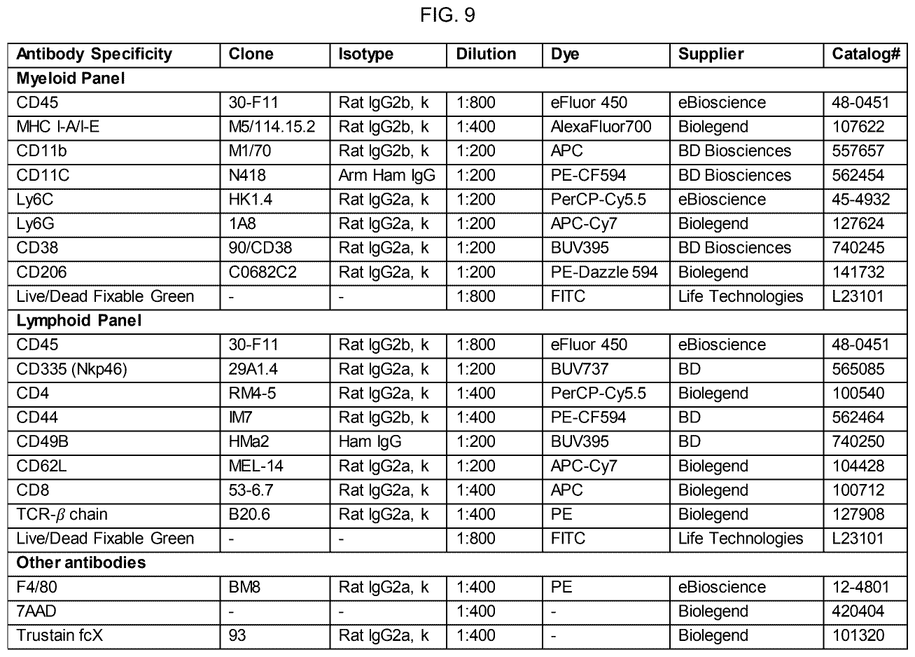

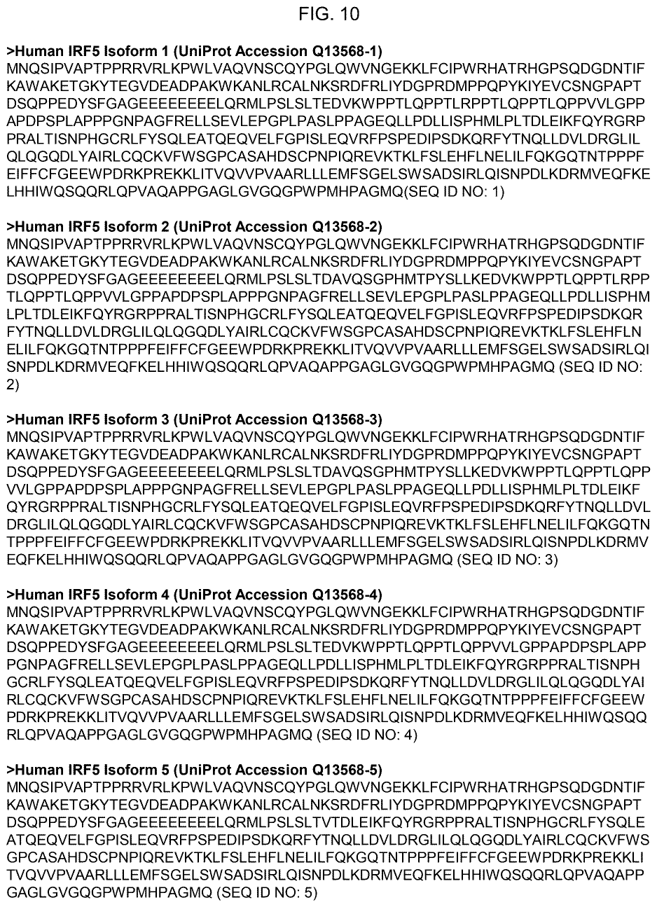

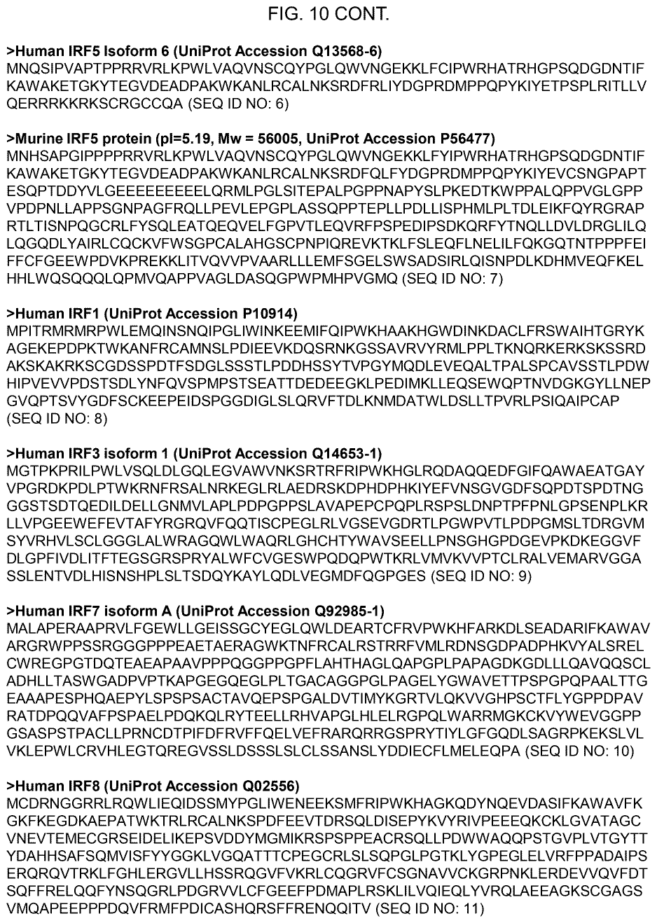

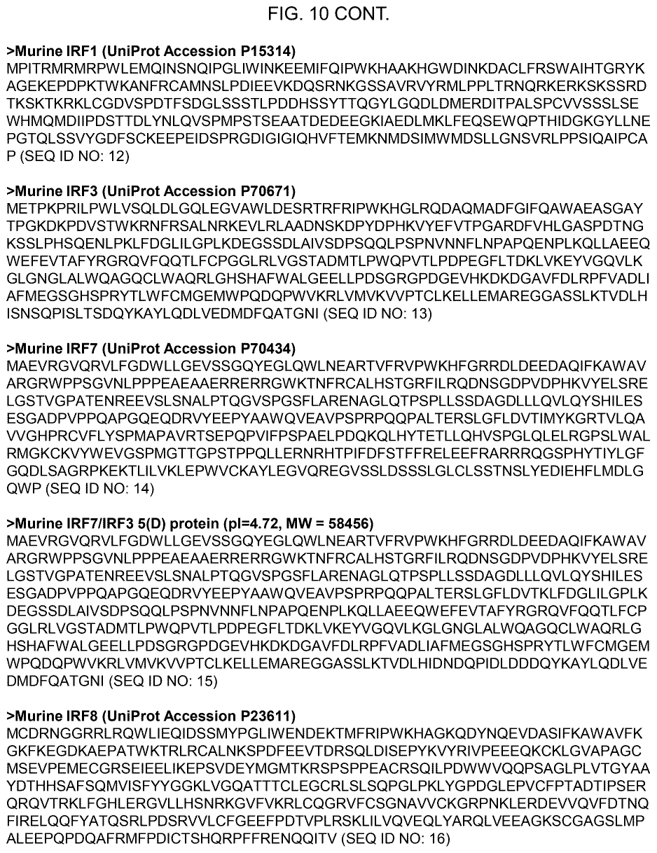

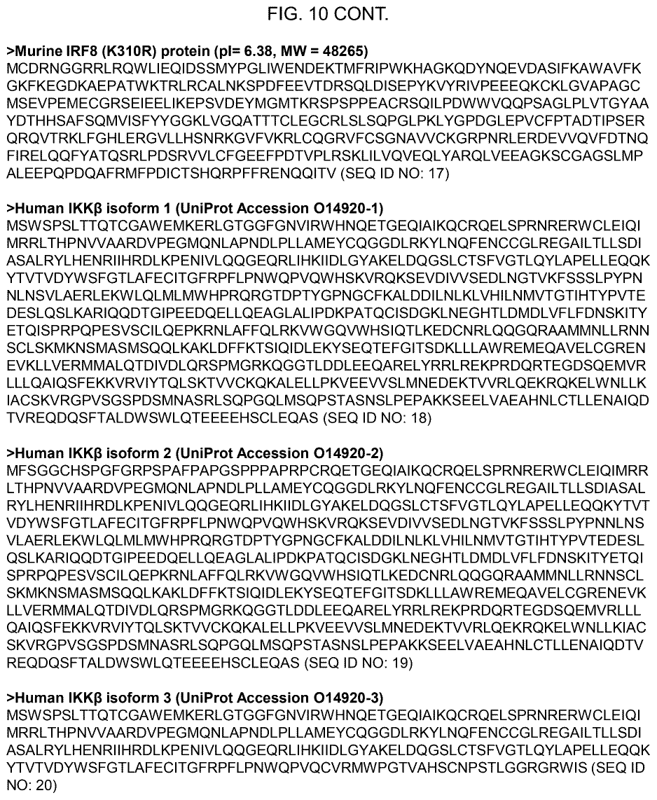

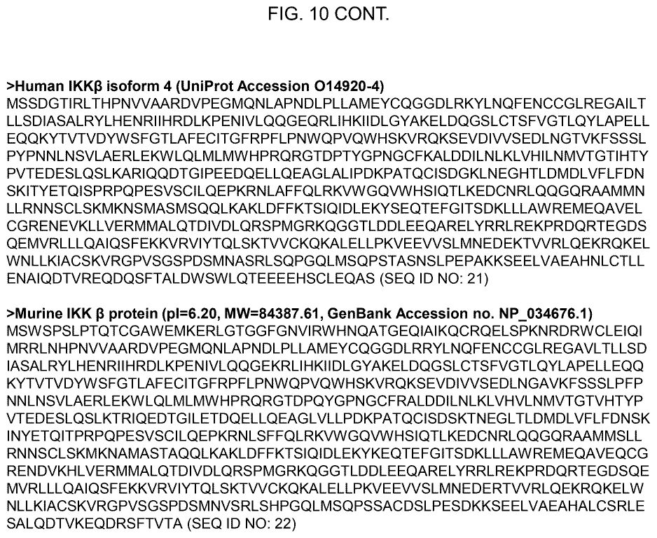

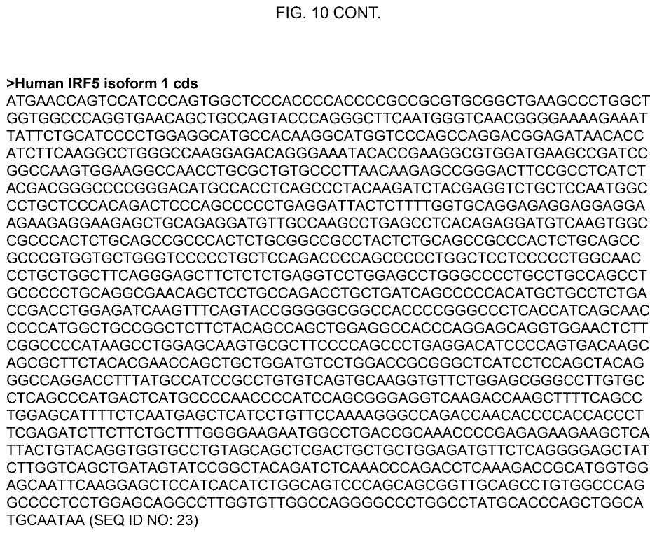

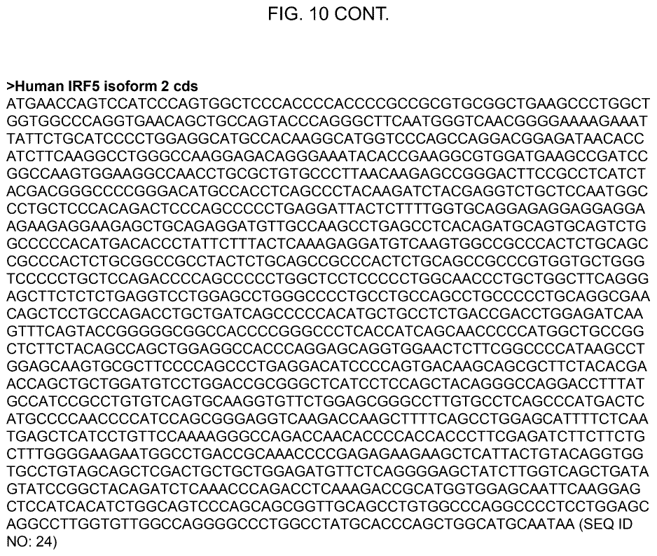

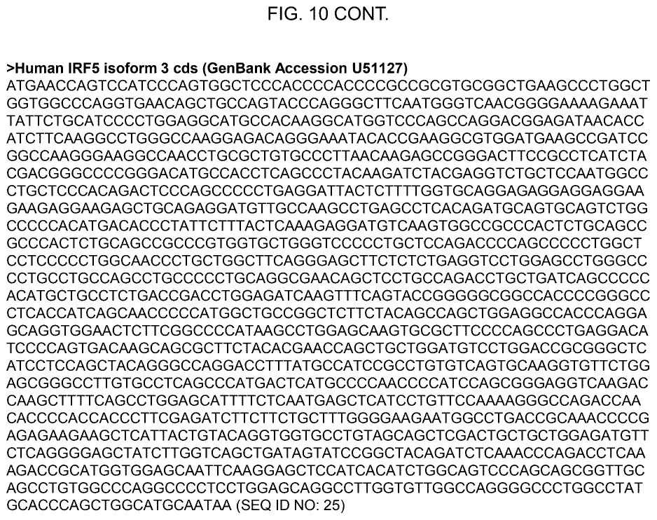

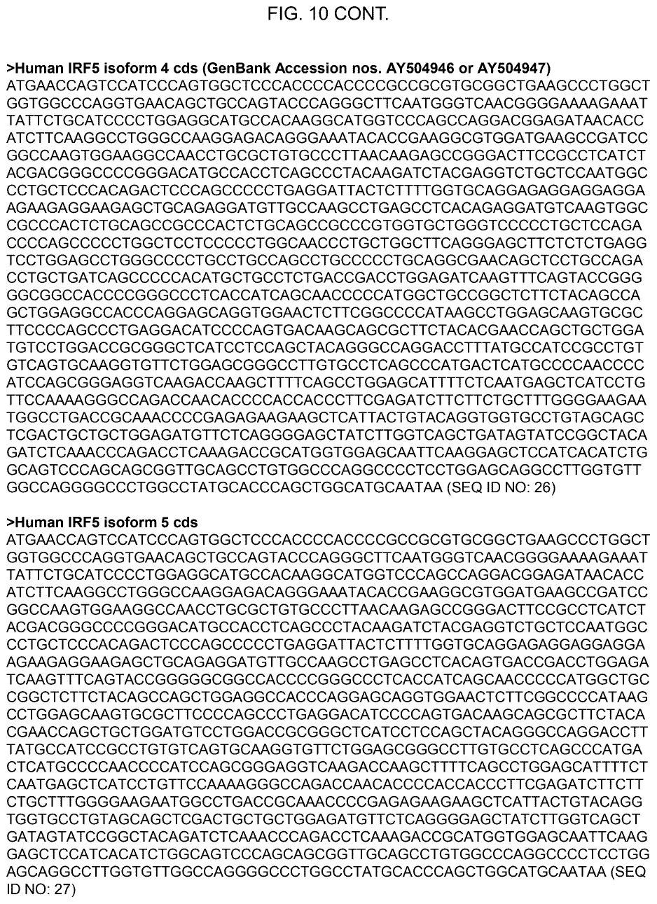

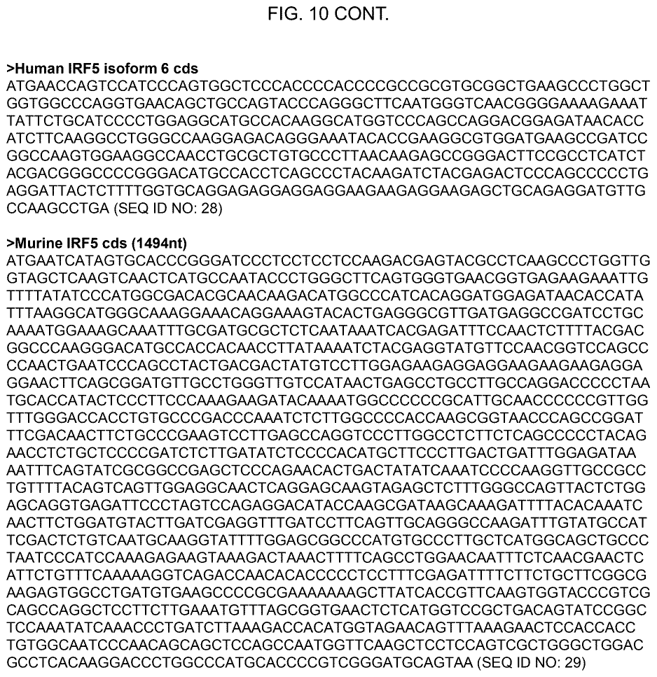

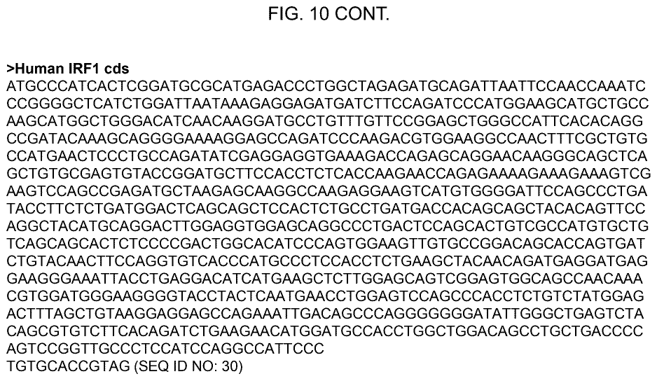

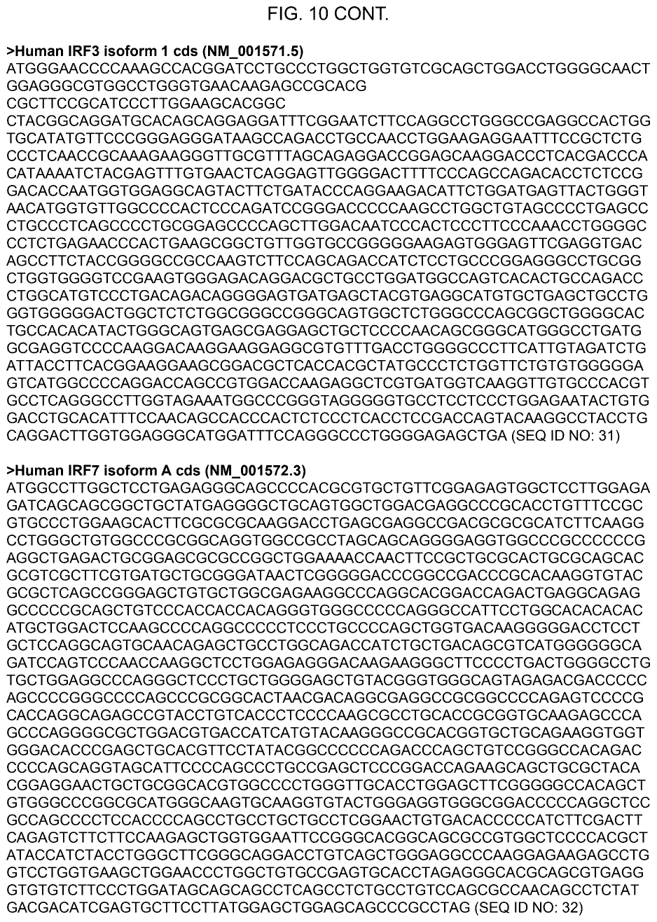

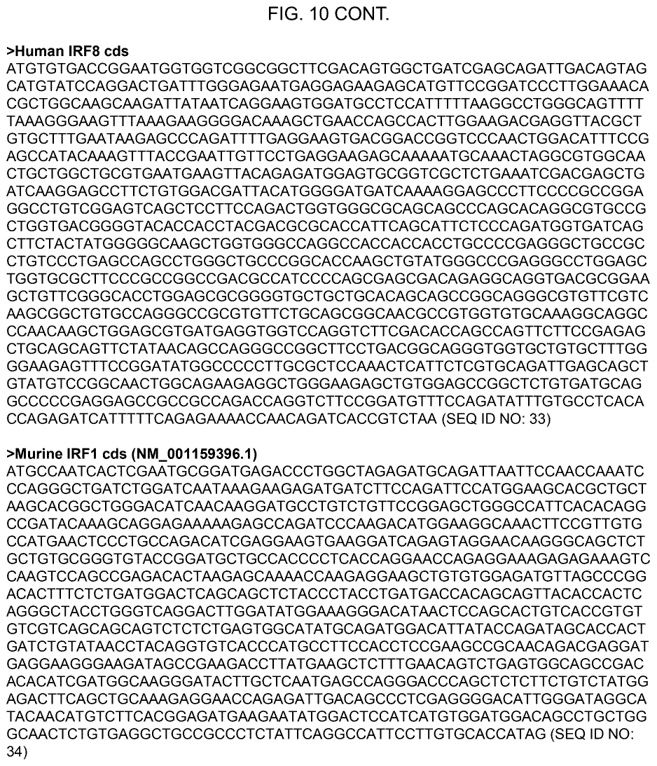

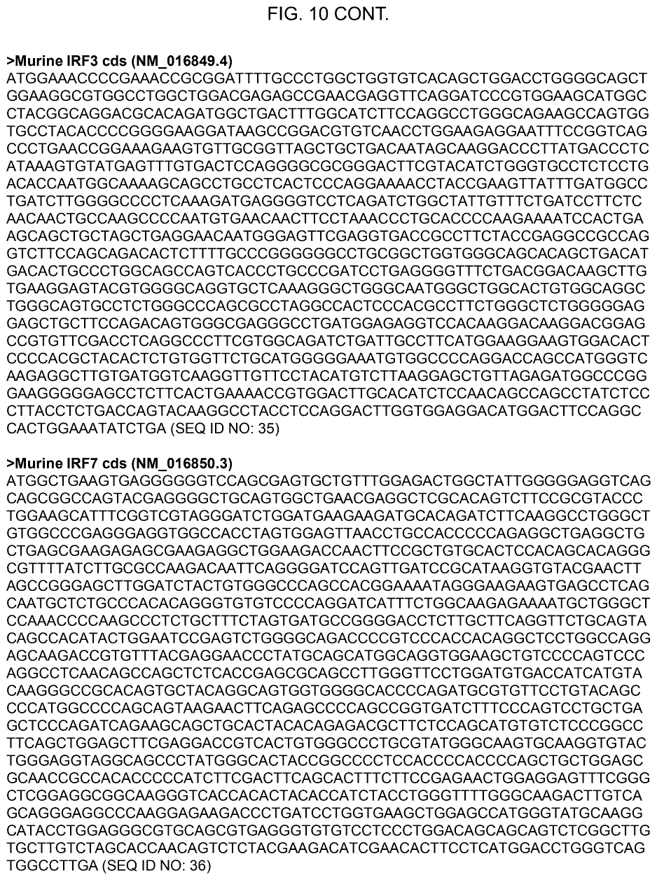

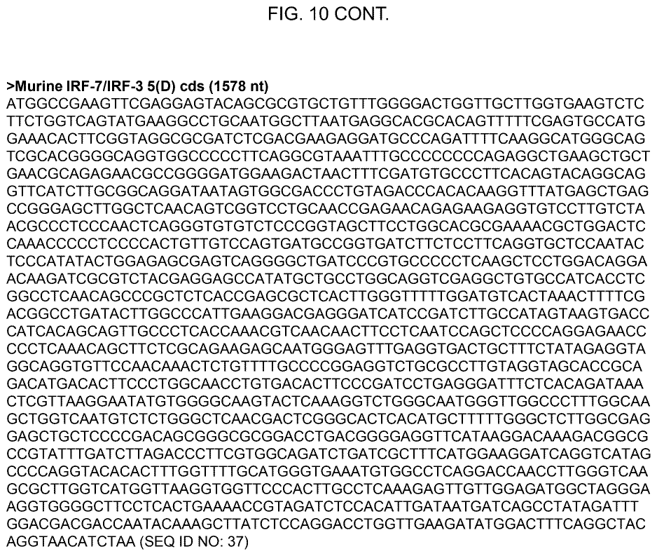

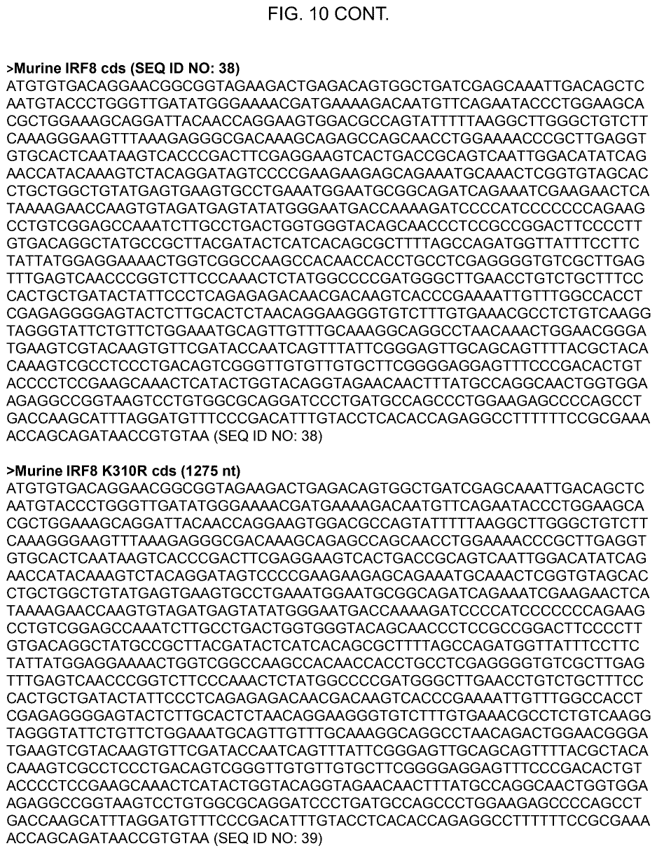

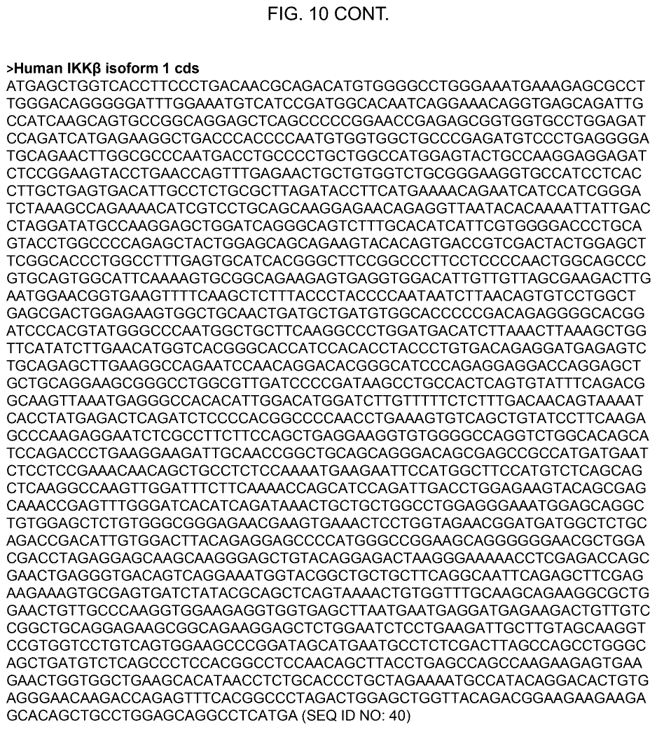

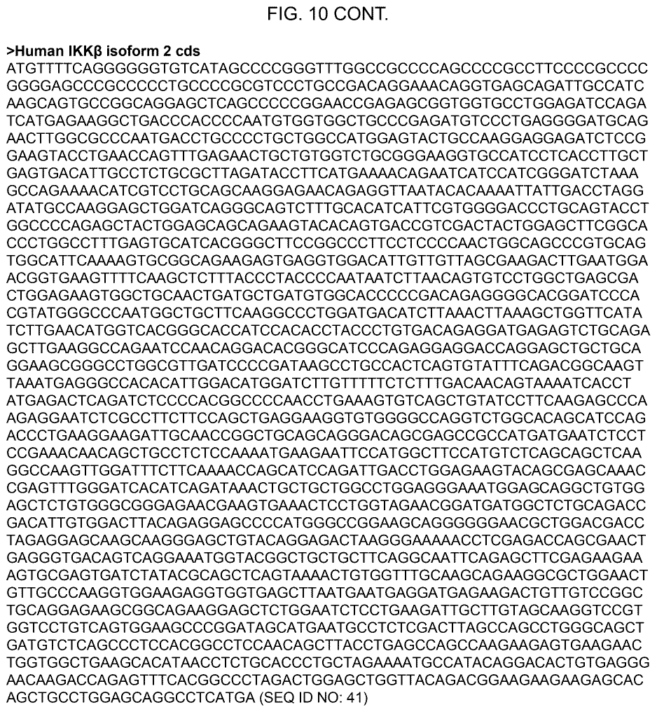

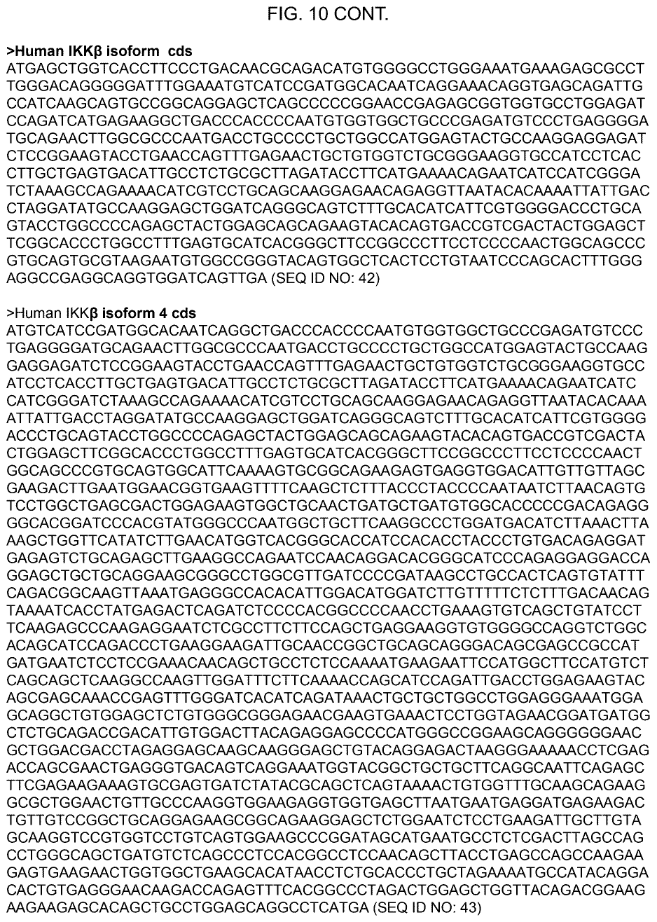

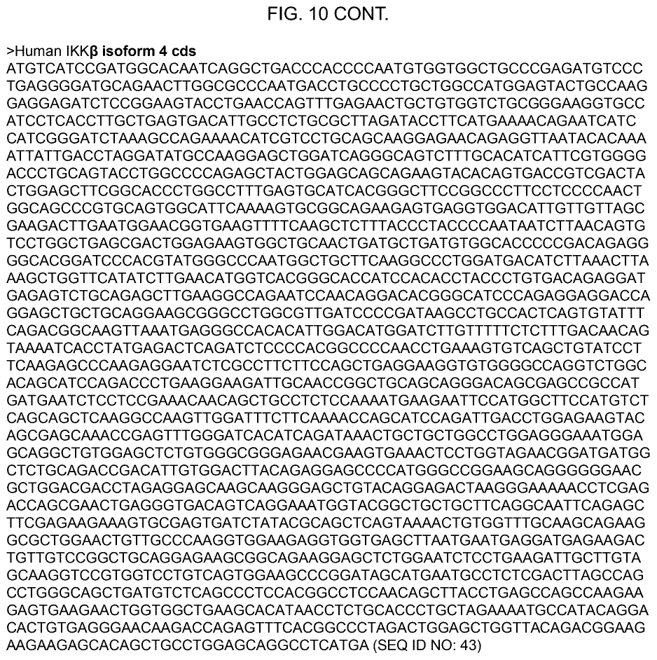

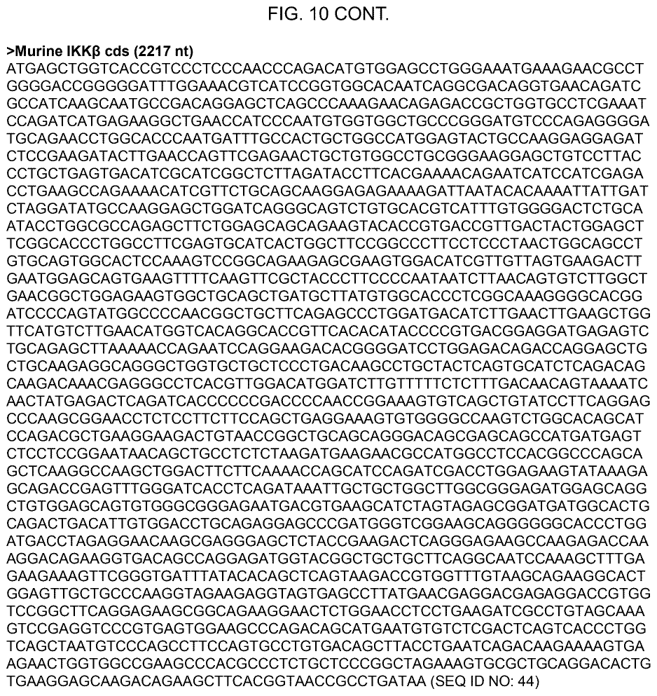

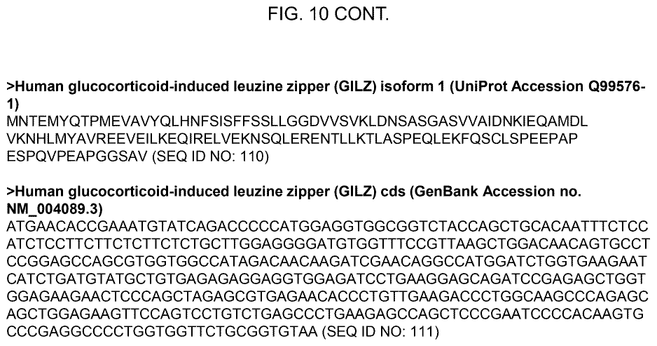

Flow Cytometry and cell sorting. Cells obtained from spleen, blood, peritoneal lavage, and bronchoalveolar lavage were analyzed by flow cytometry with myeloid and lymphoid immunophenotyping panels using the anti-mouse antibody probes listed in FIG. 9. Data were collected using a BD LSRFortessa analyzer running FACSDIVA software (Beckton Dickinson). CD11b+ and F4/80+ peritoneal macrophages were sorted using BD FACS ARIA II. All collected data were analyzed using FlowJo 10.0 software.

Cytokine analysis. Cytokine levels were evaluated using a Luminex 200 system (Luminex) at the FHCRC Immune Monitoring Shared Resource center. For ex vivo studies, cell culture supernatant was collected for the measurement of IL-6, IL-12p70, INFγ, and TNFα concentrations. For in vivo studies, plasma concentration of GM-CSF, INFγ, IL-12p70, IL-2, IL-6, and TNFα were measured.

qRT-PCR analysis. Gene expression levels were determined by qRT-PCR. To measure selected macrophage signature genes (SerpinB2, Retnla, Ccl5, Ccl11, codon-optimized IRF5, endogenous IRF5, and housekeeping GAPD genes), total RNA was isolated with RNeasy mini-columns (Qiagen) according to the manufacturer's instructions. cDNA was synthesized using a qScript cDNA Synthesis Kit (Quanta). For each sample, qRT-PCR was performed in triplicate via PerfeCTa qPCR SuperMix Low ROX (Quanta) using gene-specific probes from the Roche's Universal Probe Library (UPL) and PCR primers optimized by ProbeFinder (Roche): SerpinB2, UPL-049, F-ACTGGGGCAGTTATGACAGG (SEQ ID NO: 96), R-GATGATCGGCCACAAACTG (SEQ ID NO: 97); Retnla, UPL-078, F-TTGTTCCCTTCTCATCTGCAT (SEQ ID NO: 98), R-CCTTGACCTTATTCTCCACGA (SEQ ID NO: 99); Ccl5, UPL-105, F-CCTACTCCCACTCGGTCCT (SEQ ID NO: 100), R-CTGATTTCTTGGGTTTGCTGT (SEQ ID NO: 101); Ccl11, UPL-018, F-AGAGCTCCACAGCGCTTC (SEQ ID NO: 102), R-CAGCACCTGGGAGGTGAA (SEQ ID NO: 103); codon-optimized IRF5, UPL-022, F-TCTTAAAGACCACATGGTAGAACAGT (SEQ ID NO: 104), R-AGCTGCTGTTGGGATTGC (SEQ ID NO: 105); endogenous IRF5, UPL-011, F-GCTGTGCCCTTAACAAAAGC (SEQ ID NO: 106), R-GGCTGAGGTGGCATGTCT (SEQ ID NO: 107). Signature gene mRNA levels were normalized based on amplification of GAPD, UPL-060, F-AGCCACATCGCTCAGACAC (SEQ ID NO: 108) and R-GCCCAATACGACCAAATCC (SEQ ID NO: 109). All qRT-PCR reactions were performed using Quant Studio5 RT-PCR machines running QuantStudio6 software (Applied Biosystems). In cases when the amplification plot did not cross the threshold and no Ct value was obtained (“undetermined”), a Ct value equal to the highest cycle number of in the assay (40 cycles) was used for comparisons of relative expression.

Mice and in vivo tumor models. Except for the brain tumor model-related experiments, the mice used in these experiments were obtained from Jackson Laboratory; the others were bred and housed in the FHCRC animal facility. All of the mice were used in the context of a protocol approved by the center's Institutional Animal Care and Use Committee. To model ovarian tumors, 5×106 vascular epithelial growth factor (VEGFP)-expressing ID8 cells were injected intraperitoneally (i.p.) into 4- to 6-week-old female albino B6 (C57BL/6J-Tyr<c-2J>) mice and allowed to establish for 2 weeks. For survival studies, the animals were treated i.p. with IRF5 NPs/eGFP NPs carrying 50 μg mRNA (two doses per week for 9 weeks, or until health conditions reached euthanizing requirements). For mechanism studies, the treatments for either 1, 2, or 3 weeks, were used followed by euthanization at 48 hours following the last dose. Peritoneal lavage was performed to collect the peritoneal cells. To compare the efficacy of IRF5/IKKβ NPs with status quo macrophage targeting therapies, one group of mice received treatment with IRF5/IKKβ NPs carrying 50 μg mRNA for 3 weeks with 2 doses per week; the second received oral gavage of 15 mg/kg PI3Kγ inhibitor IPI-594 (MedKoo Biosciences Inc) formulated in vehicle (5% 1-methyl-2-pyrrolidinone in polyethylene glycol 400) daily for 3 weeks; the third group received i.p. injection of 30 mg/kg CSF1R inhibitor Pexidartinib (PLX3397, MedKoo Biosciences Inc) formulated in the same vehicle daily for 3 weeks.

To model metastatic lung cancer, 2.5×104 16F10 cells transduced with F-luc and suspended in 200 μL RPMI medium were injected into 4- to 6-week-old female albino B6 (C57BL/6J-Tyr<c-2J>) mice (Jackson Laboratories) and allowed to establish for 1 week. For survival studies, mice were treated retro-orbitally with (or without) IRF5/IKKβ or eGFP NPs carrying 30 μg mRNA suspended in PBS. Mice were treated with 3 doses/wk for 3 weeks or until health conditions reached euthanizing requirements. For mechanism studies, the mice received the same treatments for 2 weeks. Bronchoalveolar lavage was performed to collect alveolar cells for analysis.

Mice bearing glioma were generated following published protocols (Uhrbom L et al. (2004) Nat Med 10:1257-1260). Avian DF-1 cells producing RCAS-PDGFβ and RCAS-cre retroviruses were injected intracranially into both brain hemispheres (coordinates: 1 mm caudal from bregma, 2 mm lateral, depth of 2 mm from the dural surface) of Nestin-tv-a/Ink4a-arf−/−; Pten−/− mice (C57BL/6) between 4-6 weeks of age. Tumors were allowed to establish for 2 weeks. At day 15, mice received 10Gy radiation to one hemisphere, while the unirradiated hemisphere was shielded with lead. The next day, mice received retro-orbital injections of IRF5/IKKβ NPs carrying 30 μg mRNA (3 doses/wk for 3 weeks), or were assigned to the PBS control group.

In vivo bioluminescence imaging. D-Luciferin (Xenogen) in PBS (15 mg/mL) was used as a substrate for firefly luciferase imaging. Bioluminescence images were collected with a Xenogen IVIS Spectrum Imaging System (Xenogen). Mice were anesthetized with 2% isoflurane (Forane, Baxter Healthcare) before and during imaging. For ID8-VEGF ovarian tumors, each mouse was injected i.p. with 300 μg of D-Luciferin, and images were collected 10 minutes later. For B16F10 lung metastatic tumors, mice were injected i.p. with 3 mg of D-Luciferin, and images were collected 15 minutes afterwards. For brain tumor models, the mice received retro-orbital injection of 75 mg/kg body weight D-Luciferin, and images were collected 4 minutes later. Acquisition times ranged from 10 s to 5 min.

Biodistribution analysis. To determine the biodistribution of IRF5 NPs in the ID8-VEGF ovarian tumor model, mice in 7-8 groups received an i.p. or retro-orbital dose of NPs carrying 50 μg mRNA. Twenty-four hours after injection, whole blood was collected, and mice were euthanized with CO2 to retrieve organs (liver, spleen, lung, kidney, heart, intestine, pancreases, and diaphragm). All tissues were stabilized with RNAlater, then frozen on dry ice. The codon-optimized IRF5 mRNA levels in each organ were measured using RT-qPCR.

Toxicity analysis. To measure potential in vivo toxicities of repeatedly infusing macrophage-targeting NPs, we injected mice (5/group) intravenously with 6 sequential doses of IRF5/IKKβ or eGFP NPs carrying 50 μg mRNA over the course of 3 weeks. Controls received no treatment. Twenty-four hours after the final infusion, mice were anesthetized and blood was collected by retro-orbital bleed to determine the complete blood counts. Blood was also collected for serum chemistry and cytokine profile analyses (performed by Phoenix Central Laboratories, Mukilteo, WA). Animals were then euthanized with CO2 to retrieve organs, which were washed with deionized water before fixation in 4% paraformaldehyde. The tissues were processed routinely, and sections were stained with hematoxylin and eosin. The specimens were interpreted by Dr. Smitha Pillai MVSc, PhD, DACVP, a board-certified staff pathologist, in a blinded fashion.

Cytokine assays. Cytokine levels were evaluated using a Luminex 200 system (Luminex) at the FHCRC Immune Monitoring Shared Resources. For ex vivo studies, cell culture supernatant was collected for the measurement of IL-6, IL12p70, INFγ, and TNFα concentrations. For in vivo studies, we measured plasma concentrations of GM-CSF, INFγ, IL-12p70, IL-2, IL-6, and TNFα.

Statistical analysis. The statistical significance of observed differences were analyzed using the unpaired, two-tailed one-way ANOVA test. The P values for each measurement are listed in the figure or figure legends. Survival data was characterized using the Log-rank test. All statistical analyses were performed either using GraphPad Prism software version 6.0 or R software.

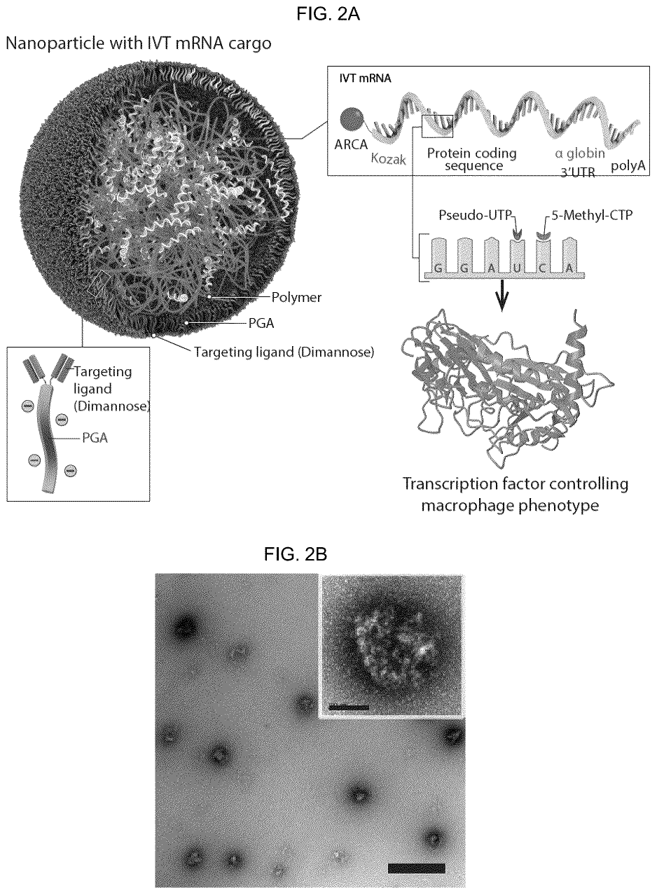

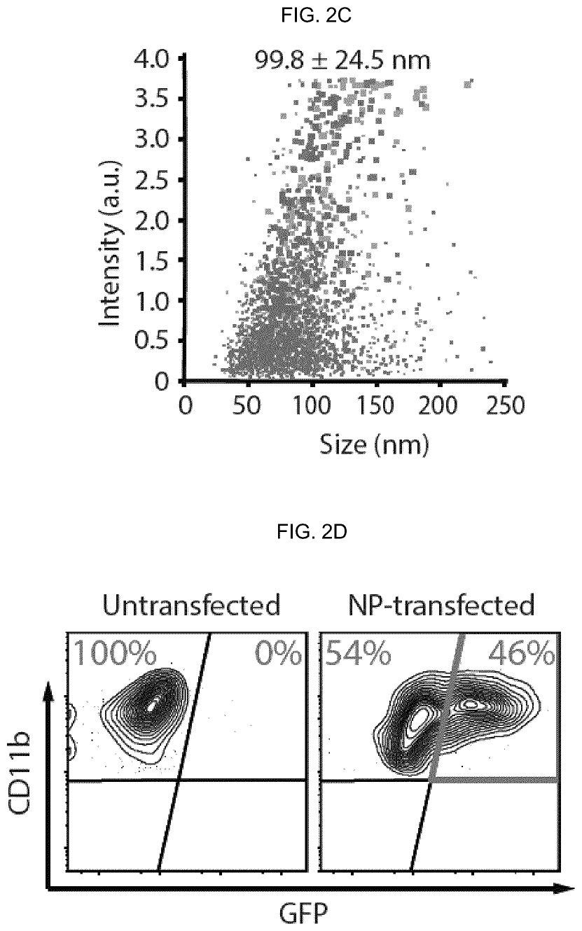

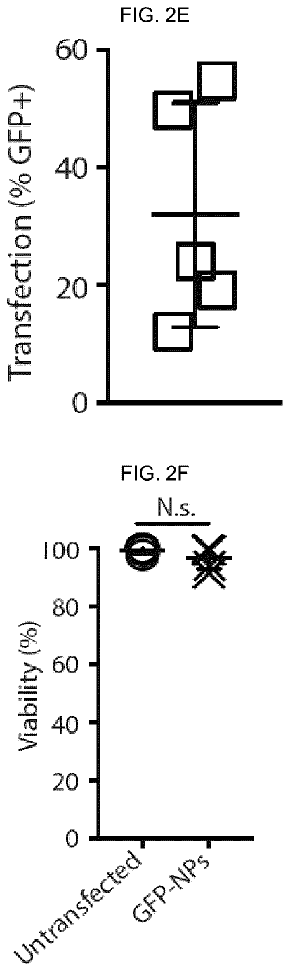

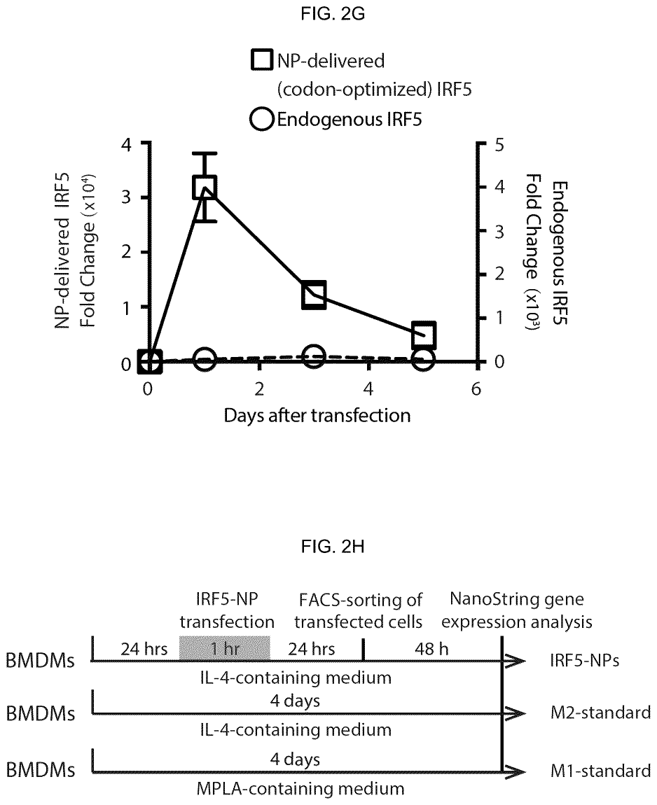

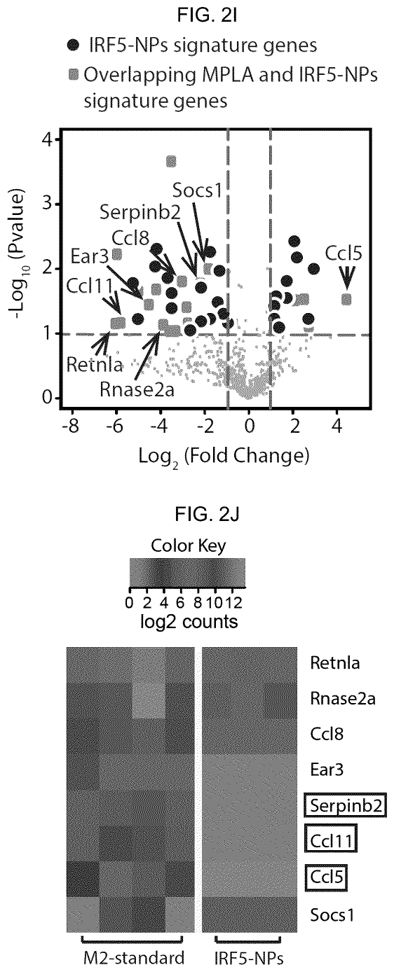

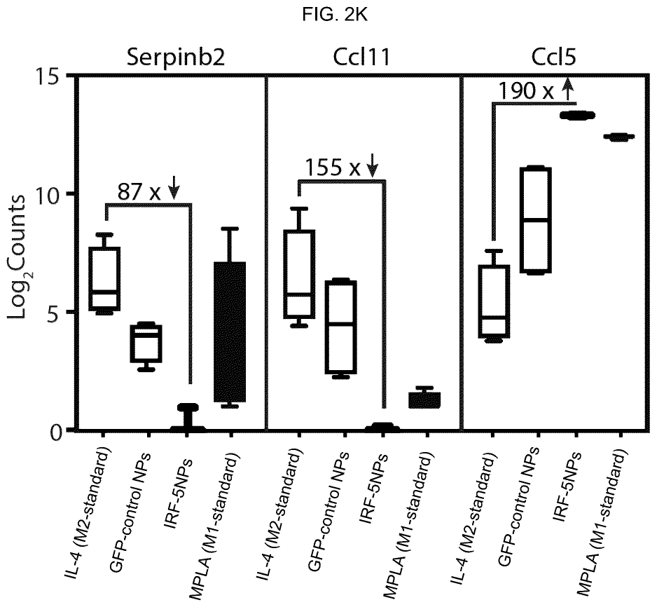

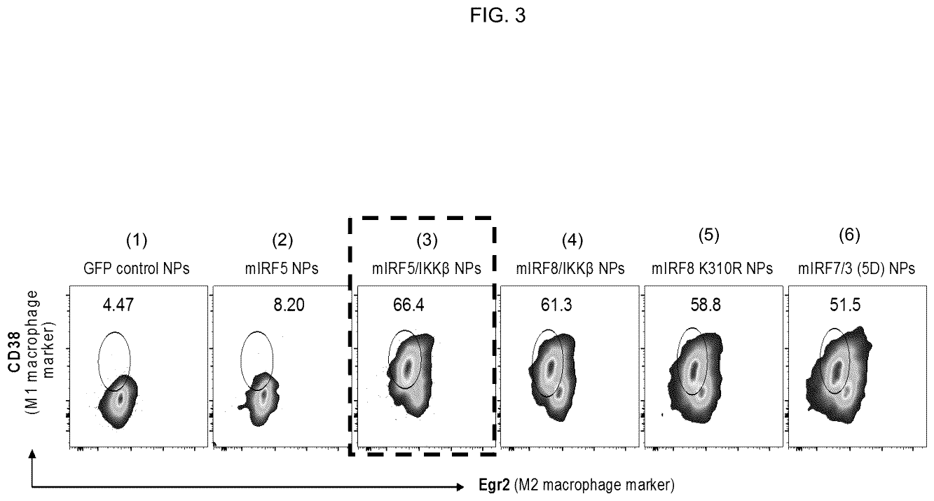

Results. Designing NPs to choreograph IVT mRNA transfection of TAMs. A targeted mRNA delivery system was developed that can introduce robust gene expression in the targeted cells by taking advantage of electrostatic interactions between cationic poly(β-amino ester) (PbAE) polymers and anionic mRNA (FIG. 2A). To improve the stability and translation of the mRNA encapsulated in the resulting nanocarriers, synthetic versions of the message were used that incorporate the modified ribonucleotides pseudouridine (Ψ) (Kariko K et al. (2008) Mol Ther 16:1833-1840) and 5-methylcytidine (m5C), and that are capped with ARCA (Anti-Reverse Cap Analog) (Quabius E S et al. (2015) N Biotechnol 32:229-235). The mRNA is released from the mRNA-PbAE complex intracellularly by hydrolytic cleavage of ester bonds in the PbAE backbone. Efficient in vivo T cell transfection was previously demonstrated using this system (Smith T T et al. (2017) Nat Nanotechnol). To target the nanoparticles to TAMs as well as further stabilize the mRNA-PbAE complexes they contain, Di-mannose moieties were engineered onto their surfaces using polyglutamic acid (PGA) as a linker (FIG. 2A). The NPs were manufactured utilizing a simple two-step, charge driven self-assembly process. First, the synthetic mRNA was complexed with a positively charged PBAE polymer, which condenses the mRNA into nano-sized complexes. This step was followed by the addition of PGA functionalized with Di-mannose, which shields the positive charge of the PBAE-mRNA particles and confers macrophage-targeting. The resulting mRNA nanocarriers had a size of 99.8±24.5 nm, a polydispersity of 0.183, and a neutral surface charge (3.40±2.15 mV ζ-potential, FIG. 2B-2C). The transfection efficiency was first tested in murine bone marrow-derived macrophages (BMDMs) using NPs formulated with green fluorescent protein-encoding mRNA (GFP-NPs). Briefly, 50,000 BMDMs were exposed to NPs containing 1 μg mRNA for 1 hour, followed by flow cytometry measurements of GFP expression the next day. Following a single NP application, we routinely transfected 31.9% (±8.5%) of these primary macrophages without reducing their viability (FIG. 2E-2F). Surface modification of particles with Di-mannose was relevant, as transfection rates with untargeted (but PGA-coated) nanocarriers dropped to an average of 25% (±2.1%) in this inherently phagocytic cell type. The NPs selectively targeted the CD11b+, F4/80+ macrophage population, with 46% of macrophages transfected and expressing high levels of eGFP (FIG. 2D). This high transfection efficiency demonstrates the potency of the disclosed systems and methods in targeted delivery of mRNA to TAMs. Based on the results of an in vitro screen for transcription factor candidates that induce macrophage polarization, two mRNAs were selected for inclusion in the NP: the first encodes IRF5, a key member of the IRF family that favors the polarization of macrophages toward the M1 phenotype, and the second encodes IKKβ, a kinase that phosphorylates and activates IRF5.

Programming immunosuppressive macrophages into proinflammatory phenotypes. To induce macrophage polarization, two mRNAs were selected for inclusion into the NPs: the first encodes IRF5, a key member of the interferon regulatory factor family that favors the polarization of macrophages toward the M1 phenotype (Krausgruber T et al. (2011) Nat Immunol 12:231-238); the second encodes IKKβ, a kinase that phosphorylates and activates IRF5 (Ren J et al. (2014) Proc Natl Acad Sci USA 111:17438-17443). A ratio of 3 IRF5 mRNAs to 1 IKKβ mRNA was used. Using real-time quantitative PCR specific for the NP-delivered (and codon-optimized) IRF5 mRNA, it was found that mRNA expression in macrophages was maximal at day 1, resulting in a 1,500-fold increase in IRF5 relative to endogenous factor levels (FIG. 2A). As expected, gene expression was transient but IRF5 levels remained strongly upregulated through day 3 (581-fold increased) and day 5 (87-fold increased) before returning to baseline.

To determine if IRF5/IKKβ-encoding NPs can reprogram M2 macrophages into the therapeutically desirable anti-cancer M1 phenotype, NanoString gene expression analysis was used. BMDMs were first cultured in the presence of interleukin-4 (IL-4) to induce a suppressive M2 phenotype (FIG. 2H). Following transfection with either control GFP-mRNA nanoparticles or IRF5/IKKβ mRNA-containing NPs, gene expression profiles were analyzed and compared with inflammatory macrophages, which were generated separately by exposing BMDMs to the TLR4 agonist Monophosphoryl Lipid A. Despite being cultured in suppressive IL-4-containing medium, macrophages transfected with IRF5/IKKβ mRNA NPs display gene expression profiles similar to inflammatory macrophages (FIG. 2I). Signature M2 macrophage genes, such as Serpinb2 and Ccl2 (Jablonski K et al. (2015) Plos One 10: e0145342; Varga T et al. (2016) J Immunol 196:4771-4782), were strongly downregulated while key M1 differentiation genes, such as Ccl5 (Sica A et al. (2012) J Clin Invest 122:787-795), were upregulated (FIG. 2J, 2K). These data establish that NP-mediated expression of IRF5 and its kinase skews suppressive macrophages toward a proinflammatory phenotype.

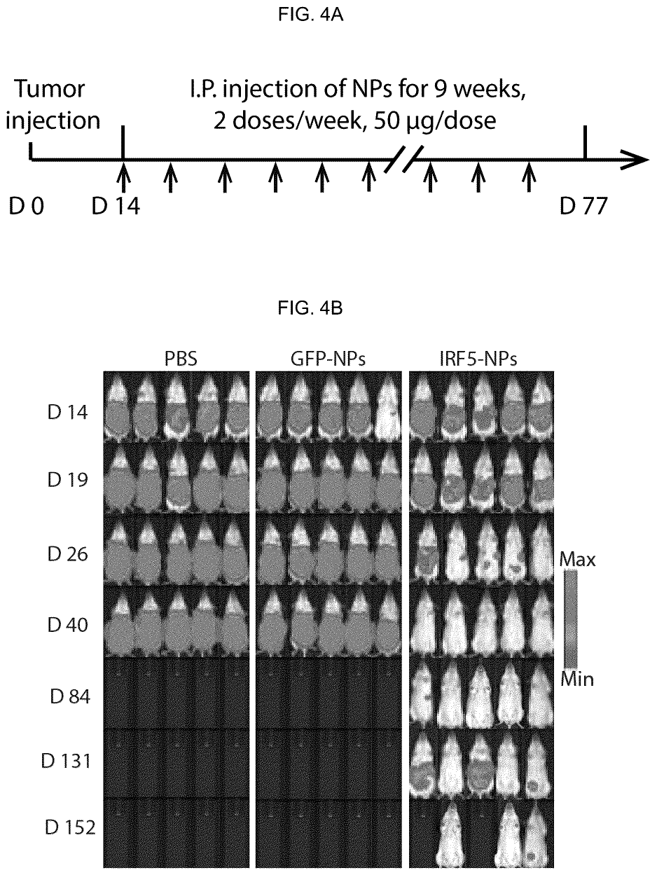

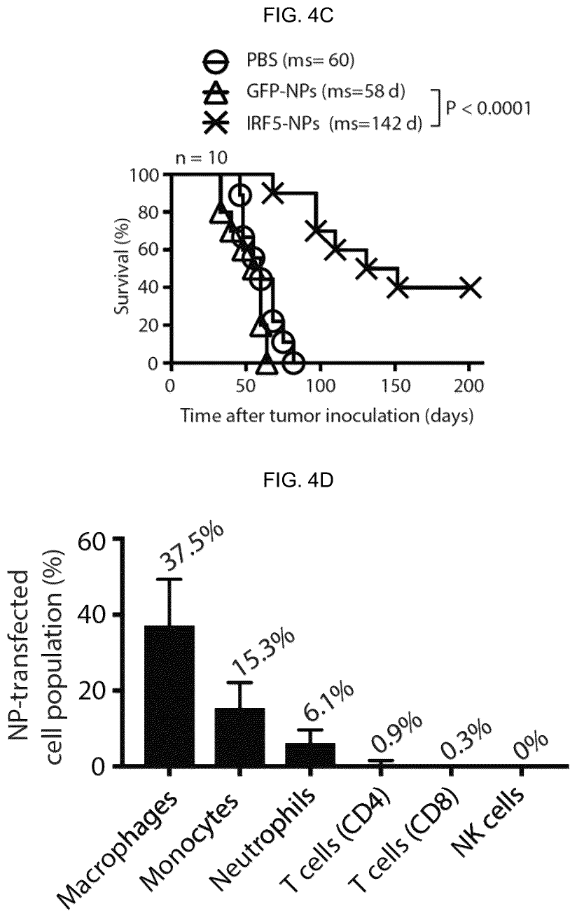

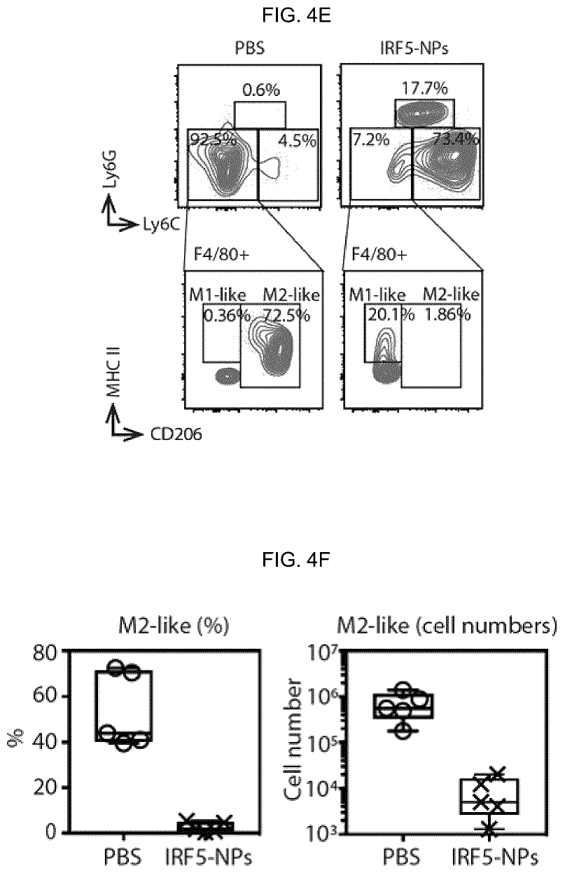

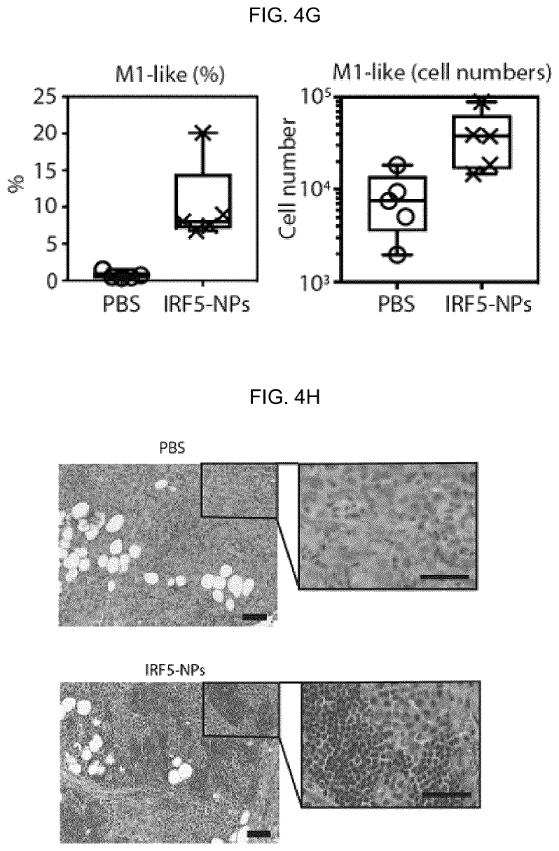

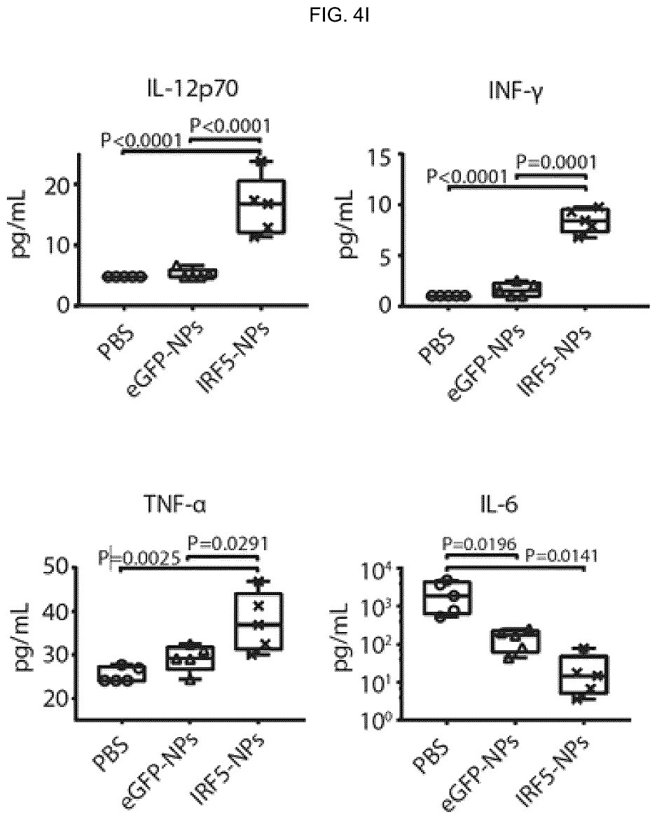

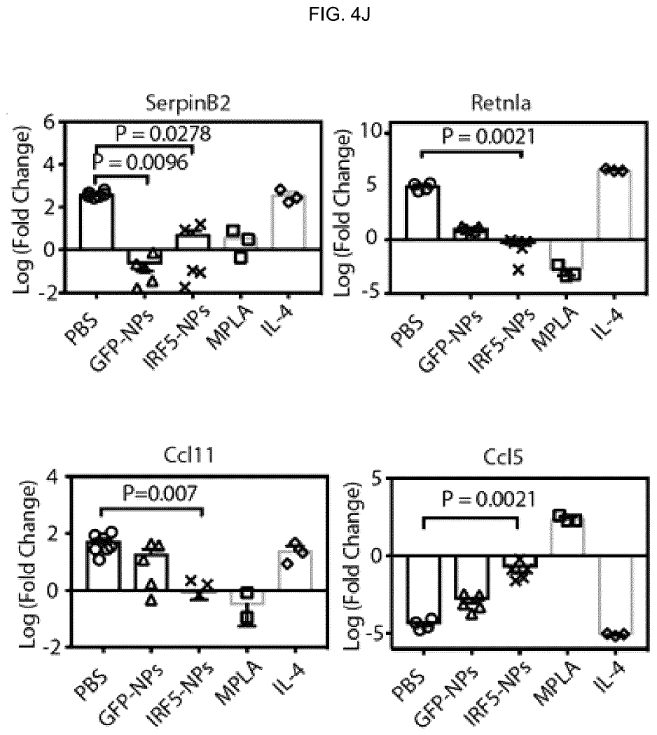

Example 2. Therapeutic effects of NP-delivered pro-M1 genes for disseminated ovarian cancer. To evaluate this treatment approach in a clinically-relevant in vivo test system, a model that recapitulates late-stage, unresectable ovarian tumors in C57BL/6 mice was used; these animals are injected with ID8 ovarian cancer cells which were tagged with luciferase to enable serial bioluminescent imaging of tumor growth (Liao J B et al. (2015) J Immunother Cancer 3:16; Stephan S B et al. (2015) Nat Biotechnol 33:97-101). The tumors were allowed to establish for two weeks. By this stage, the mice have developed nodules throughout the peritoneal wall and in the intestinal mesentery. The animals were divided into 3 groups that received PBS (control), GFPNPs (sham), or IRF5/IKKβ NP treatment at an i.p. dose of 100 μg mRNA/mouse/week for 9 weeks (FIG. 4A). It was observed that in the IRF5/IKKβ NP treated group, the disease regressed and was eventually cleared in 40% of animals (overall 142 d median survival versus 60 d in controls; FIG. 4B-4C). To understand the underlying mechanisms of IRF5/IKKβ NP-mediated anti-tumor effects, how exclusively mannose receptor-targeting confined NP interaction to phagocytes was first examined. Flow cytometry of peritoneal lavage fluid collected 24 h after the first dose of NPs targeted with Di-mannose revealed preferential gene transfer into macrophages and monocytes (average 37.1% and 15.3%, respectively, FIG. 4D), while transfection into off-target cells was low or undetectable. A detailed phenotypic and functional analysis of macrophage/monocyte populations in the peritoneum of mice with established ovarian cancer following treatment with IRF5/IKKβ nanoparticles or PBS over a 3-week period (two weekly injections) was conducted next. Flow cytometric analysis revealed that IRF5/IKKβ NPs reduced the population of immune-suppressive macrophages (Ly6C−, F4/80+, CD206+) to an average 2.6%±2.1% versus 43%±15.6% in controls (FIG. 4E-4F). Conversely, the fraction of M1-like macrophages increased from 0.5%±0.2% to 10.2%±4.1% (FIG. 4E, 4G). IRF5 gene therapy also affected the population of other immune cells. In particular, inflammatory monocytes (CD11b+, Ly6C+, Ly6G−) were more abundant (73.4%±3.6% compared to 4.5%+1.9% in untreated mice). One interesting finding in all IRF5 NP-treated animals were multifocal dense clusters of lymphocytes present within or surrounding the neoplasms (FIG. 4H), indicating that genetic programming of immune stimulatory macrophages may restore lymphocyte migration and infiltration into solid tumors.

Peritoneal macrophages were isolated by fluorescence-activated cell sorting to analyze their cytokine secretion, and detected a robust increase in the release of pro-inflammatory (anti-tumor) cytokines IL-12 (3.4-fold higher), IFN-g (8.4-fold higher), and TNF-α (1.5-fold higher), whereas the levels of IL-6, a regulatory cytokine associated with differentiation toward alternatively activated (M2-like) macrophages, were reduced by 97-fold; FIG. 4I). Genome expression profiling confirmed differentiation toward an M1-like macrophage phenotype in IRF5/IKKβ nanoparticle-treated mice. Gene expression levels of macrophages cultured ex vivo in MPLA or IL-4 were included to provide reference values for classic M1-like or M2-like macrophages, respectively (FIG. 4J).

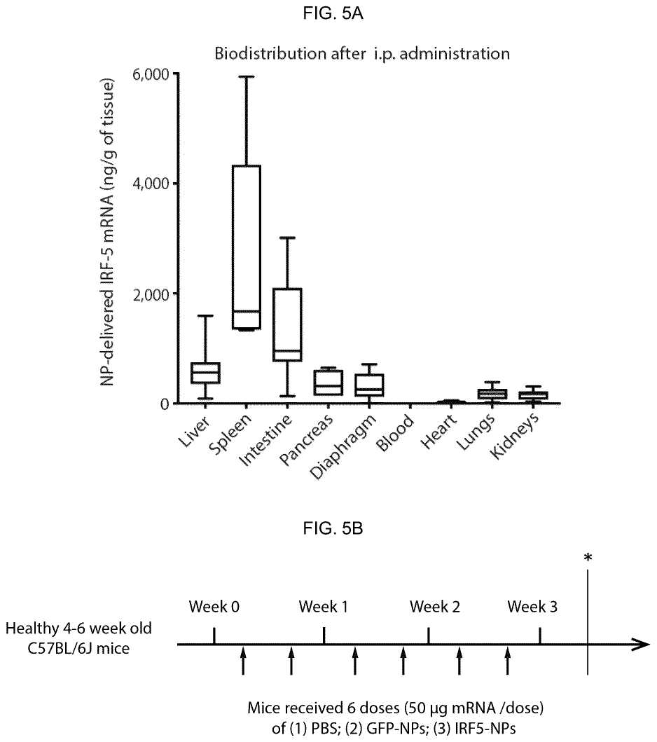

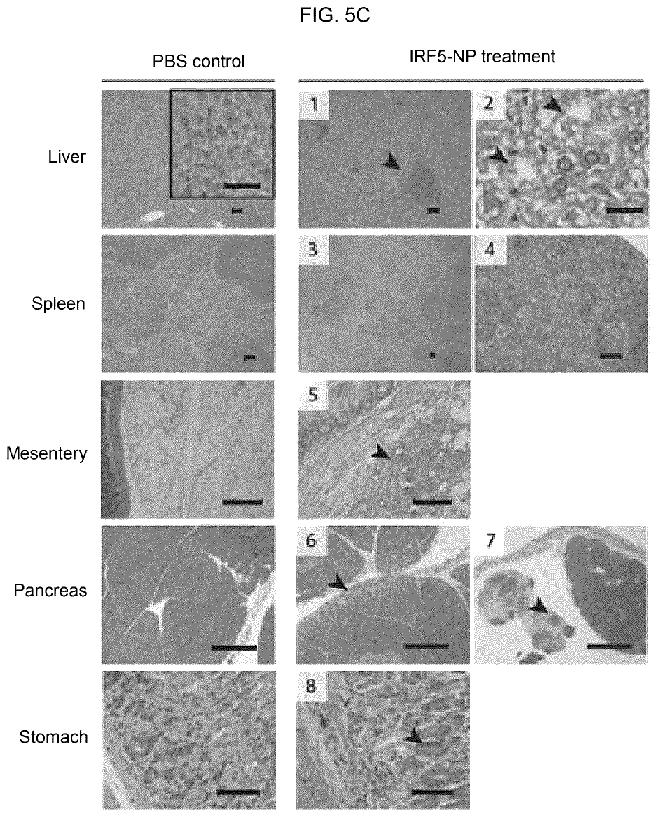

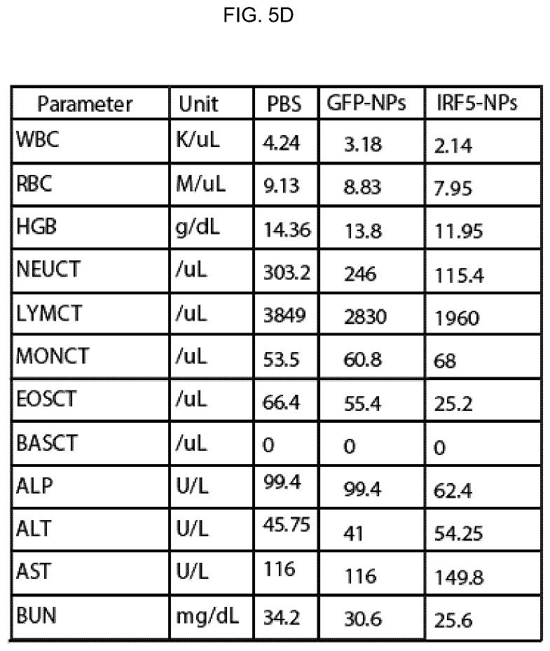

Biodistribution and safety. The distribution of nanoparticles in various organs 24 h after intraperitoneal injection using RT-qPCR assays designed to detect only nanoparticle-delivered (codon optimized) IRF5 was next quantified. The highest concentrations of IVT mRNA were found in organs located in the peritoneum, including liver, spleen, intestine, pancreas, and diaphragm (FIG. 5A). Small amounts of particle-delivered mRNA in organs that lie outside of the peritoneum (heart, lungs, kidneys) were detected, suggesting that a fraction of i.p. injected nanocarriers entered the systemic circulation. Guided by the distribution data, we next assessed whether these nanoreagents are biocompatible and safe for repeated dosing. Mice were injected with a total of 8 doses of IRF5/IKKβ NPs (two 50 μg mRNA doses/week for 4 weeks, FIG. 5B). They were euthanized 24 h after the final dose, body weight was recorded, blood was collected by retroorbital bleed for serum chemistry, and a complete gross necropsy was performed. There was no difference in body weights between groups. The following tissues were evaluated by a board certified staff pathologist: liver, spleen, mesentery, pancreas, stomach, kidney, heart, and lungs. Histopathological evaluation revealed in all cases multifocal dense clusters of lymphocytes within or surrounding tumor lesions, but no evidence of inflammation or frank necrosis was observed in tissues where neoplastic cells were not present (FIG. 5C). Also, serum chemistry of IRF5/IKKβ NP-treated mice was comparable to that of PBS controls, indicating that systemic toxicities did not occur (FIG. 5D). Because small amounts of IRF5-mRNA were detected systemically in biodistribution studies, parallel experiments were designed to quantitate inflammatory cytokines in the peripheral blood. Following a single i.p. injection of IRF5/IKKβ NPs, moderate and transient increase was measured in serum levels of interleukin-6 (IL-6) to an average of 26.8 pg/mL (FIG. 5E), and tumor necrosis factor-a (TNF-a) to an average 94.7 pg/mL (FIG. 5F). Based on previous reports, these levels are 500-fold lower than those associated with pathological findings and thus can be considered safe Tarrant J. M. (2010) Toxicol Sci 117:4-16; Copeland S et al. (2005) Clin Diagn Lab Immunol 12:60-67).

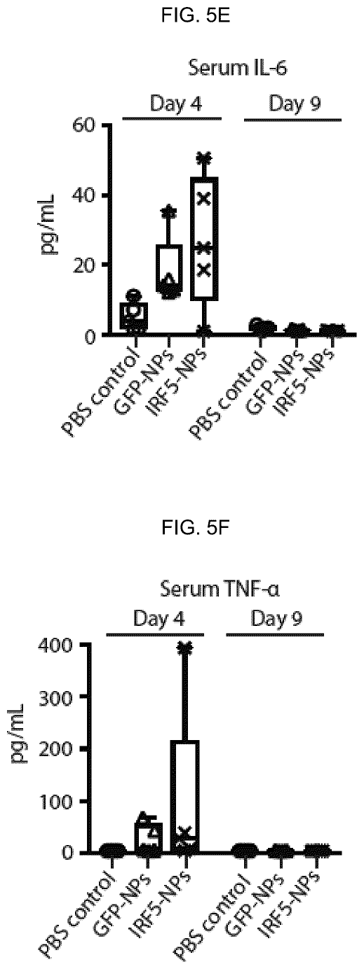

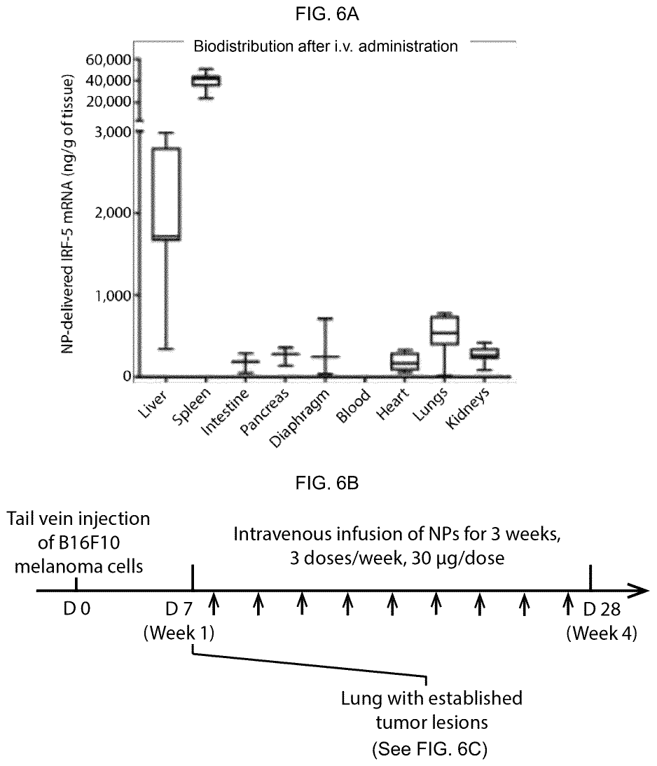

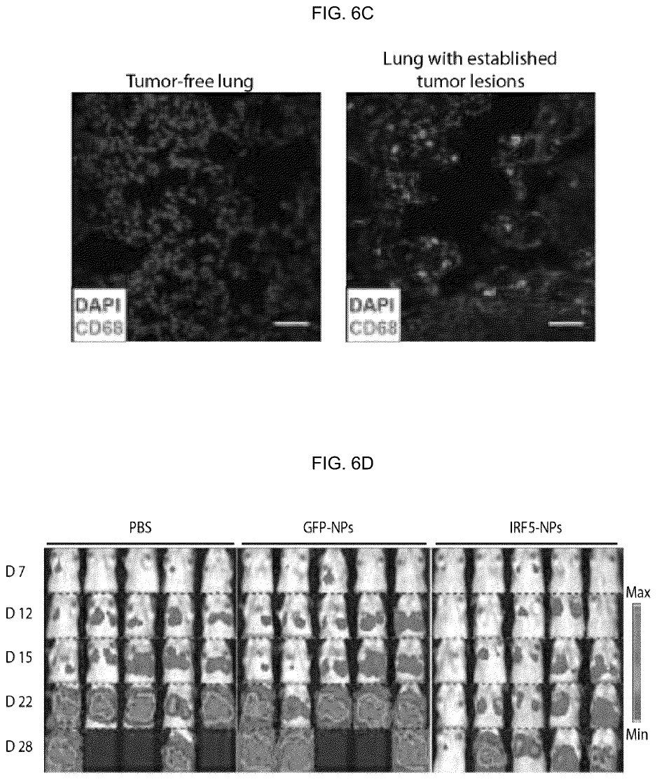

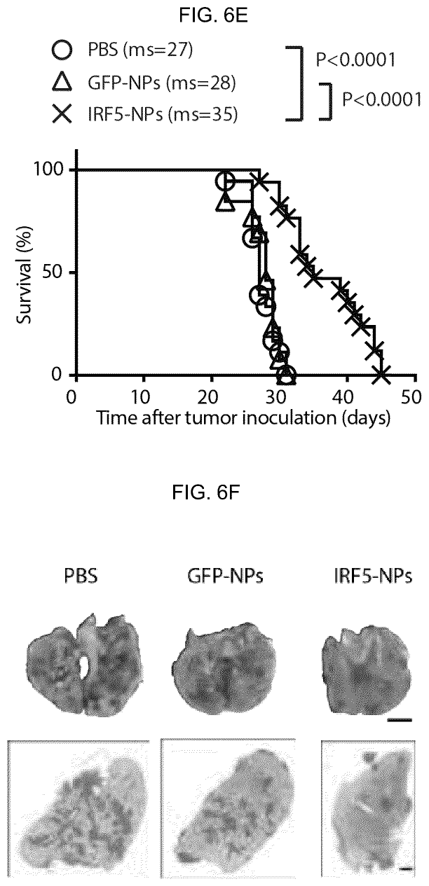

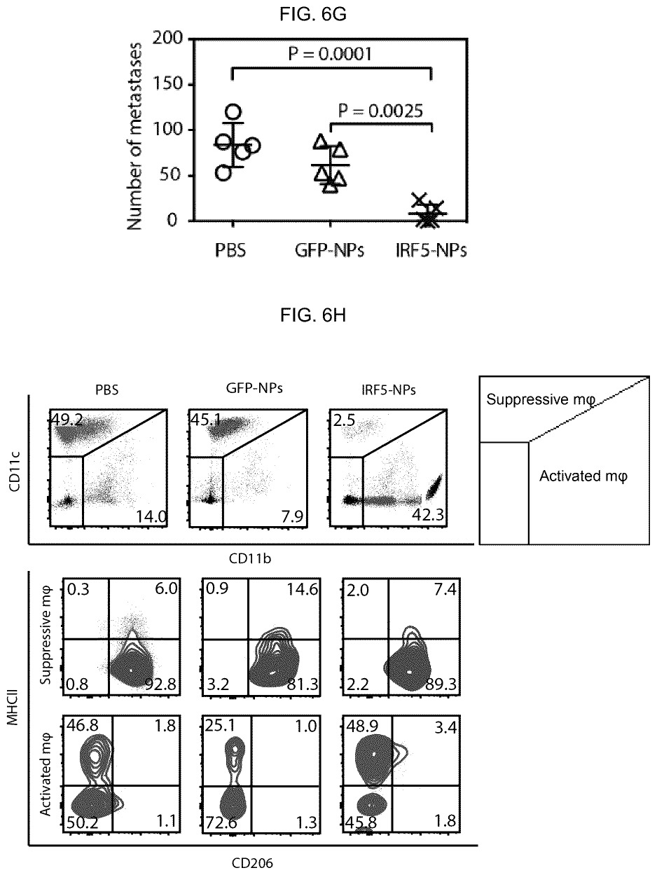

Controlling systemic tumor metastases with intravenous infusions of IRF5/IKKβ nanoparticles. Based on the therapeutic responses achieved with IRF5/IKKβ NPs administered directly into the peritoneal cavity to treat tumor lesions spread throughout the peritoneum, the next question asked was whether intravenously infused mRNA nanocarriers could program macrophages systemically to control disseminated disease. RT-qPCR biodistribution studies revealed that i.v.-infused nanocarriers preferentially deliver their mRNA cargo to organs with high levels of resident macrophages/phagocytes, mostly the spleen, liver, and lungs (FIG. 6A). To measure anti-tumor responses in a clinically relevant in vivo test system, particles containing IRF5/IKKβ mRNA were administered into mice with disseminated pulmonary melanoma metastases (FIG. 6B). Recent work describes the foundational role of monocytes and macrophages in establishing metastases caused by this disease (Butler K L et al. (2017) Sci Rep 7:45593; Nielsen S R et al. (2017) Mediators Inflamm 2017:9624760), and it was confirmed by confocal microscopy that tumor engraftment was coordinate with phagocyte accumulation in the lungs (FIG. 6C). Tumor burdens were determined by bioluminescent imaging, and mice with detectable cancers were sorted into groups that had matching levels. Groups were then randomly assigned to treatment conditions, receiving no therapy (PBS), or intravenous injections of GFP- or IRF5/IKKβ-encapsulating nanoparticles. Only IRF/IKKβ nanoparticle therapy substantially reduced tumor burdens in the lungs; in fact, they improved overall survival by a mean 1.3-fold (FIG. 6D-6E). In parallel experiments, mice were sacrificed 22 days after tumor inoculation to validate bioluminescence tumor signals with counts of pulmonary metastases and to assess macrophage polarization by flow cytometry. The total number of metastases in the lungs of IRF5/IKK NP-treated animals was 8.7-fold reduced (average 40±16 metastases) compared to PBS controls (average 419±139 metastases; FIG. 6F-6G). Flow cytometry of bronchoalveolar lavage fluid cells revealed a strong shift from immune-suppressive (CD206+, MHCII−, CD11c+, CD11blow) macrophages toward activated (CD206−, MHCII+, CD11c−, CD11b+) phagocytes (FIG. 6H-6I).

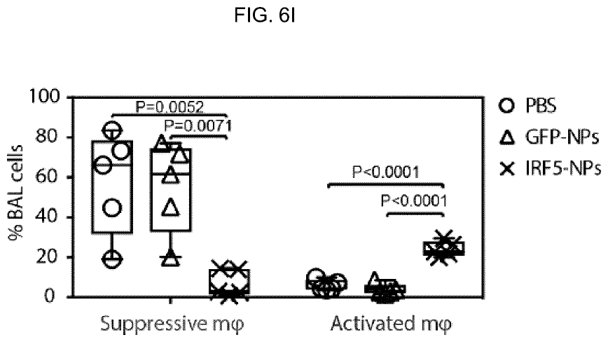

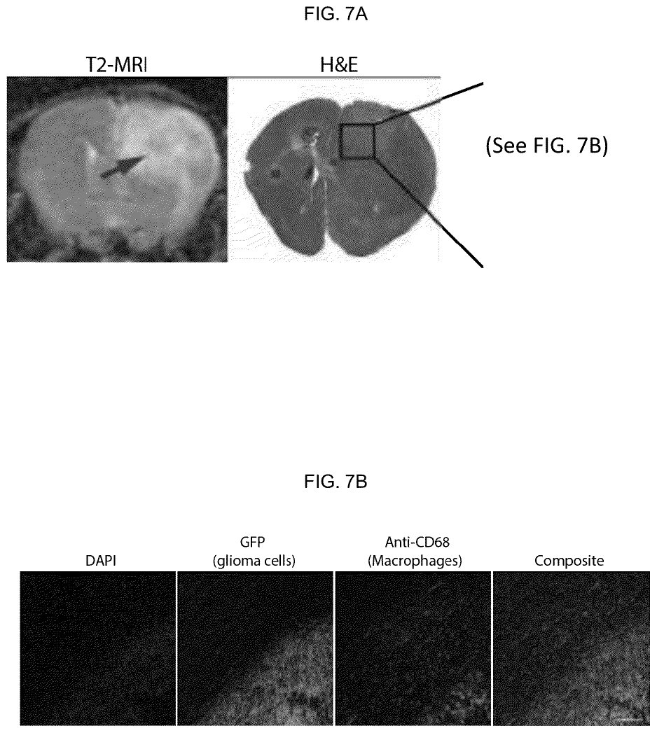

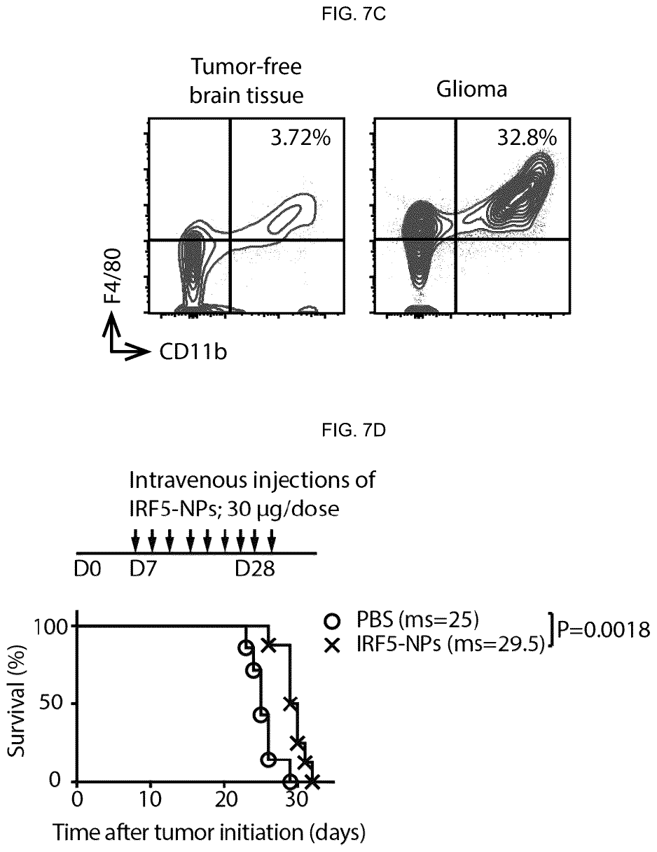

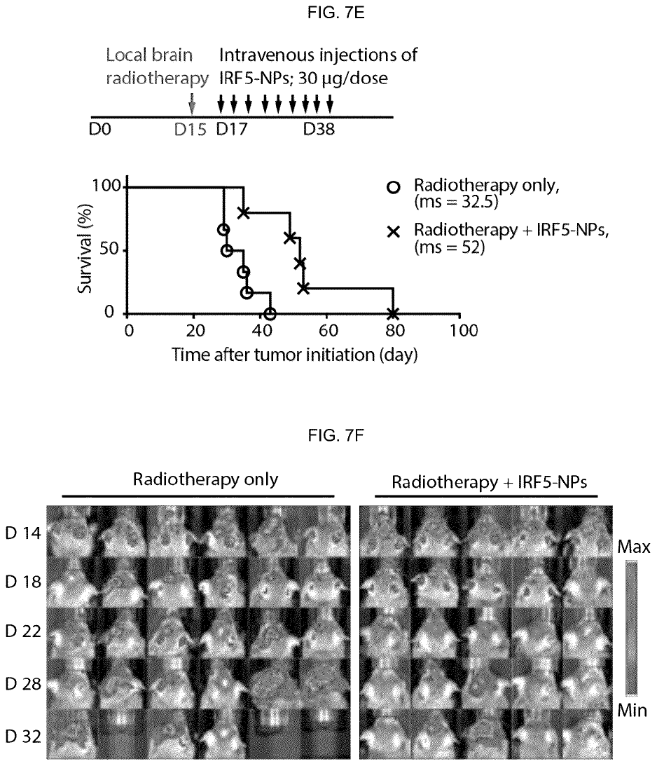

Programming tumor-suppressing phagocytes to treat glioma. For a third in vivo test system glioma was examined, which is a difficult to manage cancer type where M2-like macrophages represent the majority of non-neoplastic cells and promote tumor growth and invasion (Hambardzumyan D et al. (2016) Nat Neurosci 19:20-27). Currently, the standard of care for this disease is radiotherapy, which unfortunately offers only a temporary stabilization or reduction of symptoms and extends median survival by 3 months (Mann J et al. (2017) Front Neurol 8:748). To recapitulate the genetic events and subsequent molecular evolution of the disease, the RCAS-PDGF-B/Nestin-Tv-a; Ink4a/Arf−/−; Pten−/− transgenic mouse model of PDGFB-driven glioma (PDG mice (Hambardzumyan D et al. (2009) Transl Oncol 2:89-95; Quail D F et al. (2016) Science 352: aad3018)) was used. Brain tissue was stereotactically injected with a mixture of DF-1 cells transfected with either RCAS-PDGFβ or RCAS-cre retrovirus (1:1 mixture, 2 μL). Overexpression of the PDGFβ oncogene and the absence of the tumor suppressor genes Ink4a-arf and Pten in glioma progenitors led to the formation of 4-5 mm diameter tumors (FIG. 7A) with a nearly complete penetrance within 21 days (as established previously (Hambardzumyan D et al. (2009) Transl Oncol 2:89-95)). Using immunofluorescence, the presence of tumor-infiltrating (CD68+) macrophages (FIG. 7B, indicated in third panel from the left) were confirmed in established gliomas (shown in second panel from the left). Flow cytometry revealed that the F4/80+, CD11b+ macrophage population accounted for 32.8% of total cells in the tumor, which is 9-fold higher than seen in age-matched healthy control mice (3.7%) (FIG. 7C). The PDG mice in the experiments express the firefly luciferase gene linked to a key cancer gene promoter. Bioluminescence from this reporter has been demonstrated to be positively correlated with tumor grade (Uhrbom L et al. (2004) Nat Med 10:1257-1260), so it was used to monitor tumor development every four days after the onset of treatment. IRF/IKKβ NPs as a monotherapy was first tested: PDG mice received intravenous infusions of 9 doses of NPs loaded with IRF5/IKKβ mRNA, or PBS in the control group (3 doses/week for 3 weeks). IRF/IKKβ NP treatments only modestly suppressed tumor progression (producing on average only a 5-day survival advantage compared to untreated controls; FIG. 7D). However, combining radiotherapy as the standard-of-care with IRF5/IKKβ NP injections substantially reduced tumor growth and more than doubled the survival of treated mice compared to the PBS control group (52 d versus 25 days, respectively; FIG. 7E-7F).

In conclusion, in vivo results from three preclinical solid tumor models demonstrate that nanoparticles, administered either locally or systemically, can deliver genes encoding master regulators of macrophage polarization to re-program immunosuppressive macrophages into tumor-clearing phenotypes.

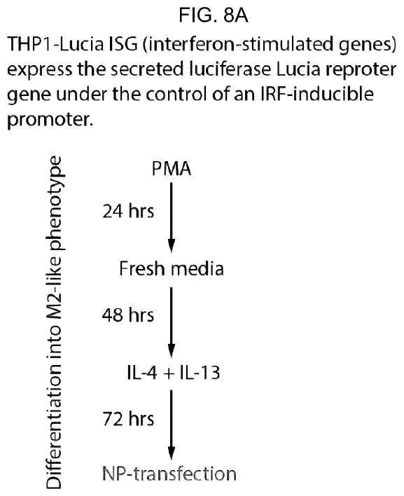

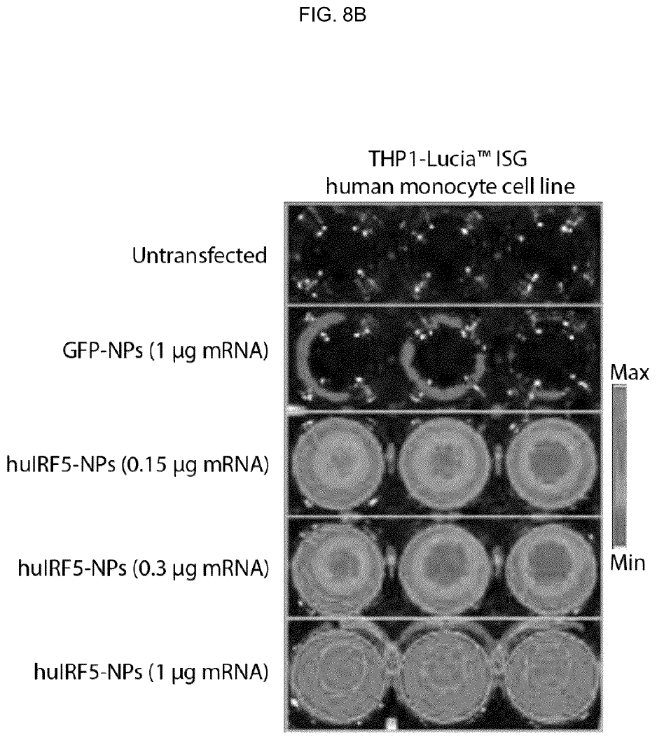

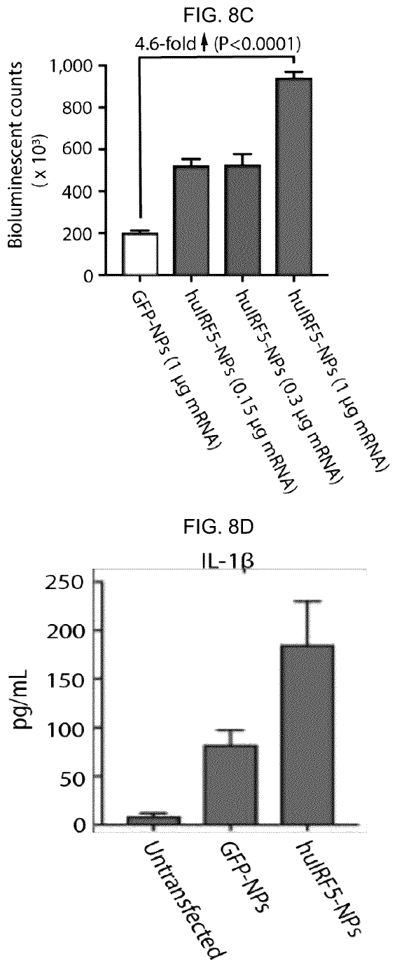

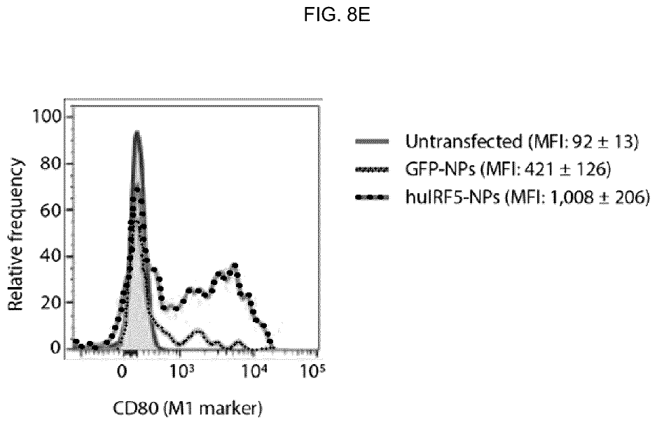

Translation from murine to human macrophages. To confirm that the data acquired in mice has relevance to treat human disease, NPs delivering IVT mRNA encoding human IRF5 and IKKβ (hulRF5 NPs) were fabricated. The human monocytic cell line THP-1 was used as a well-established M1 and M2 macrophage polarization model to test these nanocarriers (Li C et al. (2016) Sci Rep 6:21044; Surdziel E et al. (2017) Plos One 12: e0183679). M2-type macrophages were generated by treating THP-1 cells with PMA and polarizing them with IL-4 and IL-13 (FIG. 8A). To confirm that hulRF5 NPs are functional and activate the IRF pathway, THP1-Lucia™ ISG cells were transfected with nanoparticles loaded with hulRF5/IKKβ or GFP control mRNAs. THP1-Lucia™ ISG cells secrete the fluorescent Lucia reporter under the control of an IRF-inducible promoter. This composite promoter is includes five IFN-stimulated response elements (ISRE) fused to an ISG54 minimal promoter, which is unresponsive to activators of the NF-κB or AP-1 pathways. As a result, THP1-Lucia™ ISG cells allow the monitoring of the IRF pathway by determining the activity of the Lucia luciferase. It was found that hulRF5 NPs strongly upregulated luciferase expression in M2-polarized THP-1 cells, indicating that the mRNA constructs are functional in human cells (FIG. 8B-8C). To determine whether IRF5 pathway activation can reprogram M2-polarized THP-1 cells toward an M1-like phenotype, secretion of the pro-inflammatory cytokine IL-1β following NP transfection was measured. Production of IL-1β was significantly increased in THP-1 cells transfected with hulRF5 NPs versus untransfected controls (mean 21-fold; P<0.0001, FIG. 8D), which correlated with a robust upregulation (10.9-fold increased MFI, P<0.0001) of the M1 macrophage cell surface marker CD80 (FIG. 8E).

### SEQ ID NO: KEY

The nucleic acid sequences described herein are shown using standard letter abbreviations for nucleotide bases, as defined in 37 C.F.R. § 1.822. Only one strand of each nucleic acid sequence is shown, but the complementary strand is understood as included in embodiments where it would be appropriate. SEQ ID NOs: 55, 58, 61, 64, 71, 73 and 79 are not used in this sequence listing. The accompanying Sequence Listing shows the following sequences:

| SEQ ID NO: | Description |
|---|---|
| SEQ ID NO: 1 | Human IRF5 Isoform 1 (UniProt Accession |
| | Q13568-1) |
| SEQ ID NO: 2 | Human IRF5 Isoform 2 (UniProt Accession |
| | Q13568-2) |
| SEQ ID NO: 3 | Human IRF5 Isoform 3 (UniProt Accession |
| | Q13568-3) |
| SEQ ID NO: 4 | Human IRF5 Isoform 4 (UniProt Accession |
| | Q13568-4) |
| SEQ ID NO: 5 | Human IRF5 Isoform 5 (UniProt Accession |
| | Q13568-5) |
| SEQ ID NO: 6 | Human IRF5 Isoform 6 (UniProt Accession |
| | Q13568-6) |
| SEQ ID NO: 7 | Murine IRF5 protein (UniProt Accession |
| | P56477) |
| SEQ ID NO: 8 | Human IRF1 (UniProt Accession P10914) |
| SEQ ID NO: 9 | Human IRF3 isoform 1 (UniProt Accession |
| | Q14653-1) |
| SEQ ID NO: 10 | Human IRF7 isoform A (UniProt Accession |
| | Q92985-1) |
| SEQ ID NO: 11 | Human IRF8 (UniProt Accession Q02556) |
| SEQ ID NO: 12 | Murine IRF1 (UniProt Accession P15314) |
| SEQ ID NO: 13 | Murine IRF3 (UniProt Accession P70671) |
| SEQ ID NO: 14 | Murine IRF7 (UniProt Accession P70434) |
| SEQ ID NO: 15 | Murine IRF7/IRF3 5(D) protein |
| SEQ ID NO: 16 | Murine IRF8 (UniProt Accession P23611) |
| SEQ ID NO: 17 | Murine IRF8 (K310R) protein |
| SEQ ID NO: 18 | Human IKKβ isoform 1 (UniProt Accession |
| | O14920-1) |
| SEQ ID NO: 19 | Human IKKβ isoform 2 (UniProt Accession |
| | O14920-2) |
| SEQ ID NO: 20 | Human IKKβ isoform 3 (UniProt Accession |
| | O14920-3) |
| SEQ ID NO: 21 | Human IKKβ isoform 4 (UniProt Accession |
| | O14920-4) |
| SEQ ID NO: 22 | Murine IKK β protein (GenBank Accession |
| | no. NP_034676.1) |
| SEQ ID NO: 23 | Human IRF5 isoform 1 cds |
| SEQ ID NO: 24 | Human IRF5 isoform 2 cds |
| SEQ ID NO: 25 | Human IRF5 isoform 3 cds (GenBank |
| | Accession U51127) |
| SEQ ID NO: 26 | Human IRF5 isoform 4 cds (GenBank |
| | Accession nos. AY504946 or AY504947) |
| SEQ ID NO: 27 | Human IRF5 isoform 5 cds |
| SEQ ID NO: 28 | Human IRF5 isoform 6 cds |
| SEQ ID NO: 29 | Murine IRF5 cds |
| SEQ ID NO: 30 | Human IRF1 cds |
| SEQ ID NO: 31 | Human IRF3 isoform 1 cds (NM_001571.5) |
| SEQ ID NO: 32 | Human IRF7 isoform A cds (NM_001572.3) |
| SEQ ID NO: 33 | Human IRF8 cds |
| SEQ ID NO: 34 | Murine IRF1 cds (NM_001159396.1) |
| SEQ ID NO: 35 | Murine IRF3 cds (NM_016849.4) |
| SEQ ID NO: 36 | Murine IRF7 cds (NM_016850.3) |
| SEQ ID NO: 37 | Murine IRF-7/IRF-3 5(D) cds |
| SEQ ID NO: 38 | Murine IRF8 cds |
| SEQ ID NO: 39 | Murine IRF8 K310R cds |
| SEQ ID NO: 40 | Human IKKβ isoform 1 cds |
| SEQ ID NO: 41 | Human IKKβ isoform 2 cds |
| SEQ ID NO: 42 | Human IKKβ isoform cds |
| SEQ ID NO: 43 | Human IKKβ isoform 4 cds |
| SEQ ID NO: 44 | Murine IKKβ cds |
| SEQ ID NO: 45 | CDRH1 that binds CD206 |
| SEQ ID NO: 46 | CDRH2 that binds CD206 |
| SEQ ID NO: 47 | CDRH3 that binds CD206 |
| SEQ ID NO: 48 | CDRH1 that binds CD206 |
| SEQ ID NO: 49 | CDRH2 that binds CD206 |
| SEQ ID NO: 50 | CDRH3 that binds CD206 |
| SEQ ID NO: 51 | CDRH1 that binds CD206 |
| SEQ ID NO: 52 | CDRH2 that binds CD206 |
| SEQ ID NO: 53 | CDRH3 that binds CD206 |
| SEQ ID NO: 54 | CDRL1 that binds CD163 |
| SEQ ID NO: 56 | CDRL3 that binds CD163 |
| SEQ ID NO: 57 | CDRH1 that binds CD163 |
| SEQ ID NO: 59 | CDRH3 that binds CD163 |
| SEQ ID NO: 60 | CDRL1 that binds CD163 |
| SEQ ID NO: 62 | CDRL3 that binds CD163 |
| SEQ ID NO: 63 | CDRH1 that binds CD163 |
| SEQ ID NO: 65 | CDRH3 that binds CD163 |
| SEQ ID NO: 66 | CDRL1 that bind CD23 |
| SEQ ID NO: 67 | CDRL2 that bind CD23 |
| SEQ ID NO: 68 | CDRL3 that bind CD23 |
| SEQ ID NO: 69 | CDRH1 that bind CD23 |
| SEQ ID NO: 70 | CDRH2 that bind CD23 |
| SEQ ID NO: 72 | CDRL1 that bind CD38 |
| SEQ ID NO: 74 | CDRL3 that bind CD38 |
| SEQ ID NO: 75 | CDRH1 that bind CD38 |
| SEQ ID NO: 76 | CDRH2 that binds CD38 |
| SEQ ID NO: 77 | CDRH3 that binds CD38 |
| SEQ ID NO: 78 | CDRL1 that binds CD38 |
| SEQ ID NO: 80 | CDRL3 that binds CD38 |
| SEQ ID NO: 81 | CDRH1 that binds CD38 |
| SEQ ID NO: 82 | CDRH2 that binds CD38 |
| SEQ ID NO: 83 | CDRH3 that binds CD38 |
| SEQ ID NO: 84 | CDRL1 that binds CD38 |
| SEQ ID NO: 85 | CDRL2 that binds CD38 |
| SEQ ID NO: 86 | CDRL3 that binds CD38 |
| SEQ ID NO: 87 | CDRH1 that binds CD38 |
| SEQ ID NO: 88 | CDRH2 that binds CD38 |
| SEQ ID NO: 89 | CDRH3 that binds CD38 |
| SEQ ID NO: 90 | CDRL1 that binds CD64 |
| SEQ ID NO: 91 | CDRL2 that binds CD64 |
| SEQ ID NO: 92 | CDRL3 that binds CD64 |
| SEQ ID NO: 93 | CDRH1 that binds CD64 |
| SEQ ID NO: 94 | CDRH2 that binds CD64 |
| SEQ ID NO: 95 | CDRH3 that binds CD64 |
| SEQ ID NO: 96 | Forward primer SerpinB2 |
| SEQ ID NO: 97 | Reverse primer SerpinB2 |
| SEQ ID NO: 98 | Forward primer Retnla |
| SEQ ID NO: 99 | Reverse primer Retnla |
| SEQ ID NO: 100 | Forward primer Ccl5 |
| SEQ ID NO: 101 | Reverse primer Ccl5 |
| SEQ ID NO: 102 | Forward primer Ccl11 |
| SEQ ID NO: 103 | Reverse primer Cdl11 |
| SEQ ID NO: 104 | Forward primer codon optimized IRF5 |
| SEQ ID NO: 105 | Reverse primer codon optimized IRF5 |
| SEQ ID NO: 106 | Forward primer endogenous IRF5 |
| SEQ ID NO: 107 | Reverse primer endogenous IRF5 |
| SEQ ID NO: 108 | Forward primer GAPD |
| SEQ ID NO: 109 | Reverse primer GAPD |
| SEQ ID NO: 110 | Human glucocorticoid-induced leuzine zipper |
| | (GILZ) isoform 1 (UniProt Accession Q99576-1) |
| SEQ ID NO: 111 | Human glucocorticoid-induced leuzine zipper |
| | (GILZ) cds (GenBank Accession no. NM_004089.3 |

As will be understood by one of ordinary skill in the art, each embodiment disclosed herein can comprise, consist essentially of or consist of its particular stated element, step, ingredient or component. Thus, “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in the ability to treat the mouse model of ovarian cancer as described in Example 2.

Unless otherwise indicated, all numbers expressing quantities of ingredients, properties such as molecular weight, reaction conditions, and so forth used in the specification and claims are to be understood as being modified in all instances by “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, “about” has the meaning reasonably ascribed to it by a person skilled in the art when used in conjunction with a stated numerical value or range, i.e. denoting somewhat more or somewhat less than the stated value or range, to within a range of +20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

“A”, “an”, “the”, and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method of altering an activation state of immune cells in vivo comprising: administering nanoparticles comprising nucleotides encoding one or more interferon regulatory factors (IRFs) to a subject thereby altering the activation state of immune cells in vivo.

2. The method of claim 1, wherein the activation state is altered from an inactivated state to an activated state and wherein the encoded one or more IRFs comprise IRF1, IRF3, IRF5, IRF7, IRF8, an IRF7 and IRF3 fusion protein, or a combination thereof.

3. The method of claim 1, wherein the nanoparticles further comprise nucleotides encoding IKKβ.

4. The method of claim 1, wherein the immune cells comprise macrophages within a tumor.

5. The method of claim 1, wherein the nanoparticles further comprise a targeting ligand linked to a coating on the nanoparticle, wherein the targeting ligand binds CD206, CD163, or CD23.

6. The method of claim 1, wherein the nanoparticles further comprise di-mannose linked to a coating on the nanoparticle.

7. The method of claim 1, wherein the activation state is altered from an activated state to an inactivated state and wherein the encoded one or more IRFs comprises IRF4.

8. The method of claim 7, wherein the nanoparticles further comprise nucleotides encoding glucocorticoid-induced leuzine zipper (GILZ).

9. The method of claim 7, wherein the nanoparticles further comprise a targeting ligand linked to a coating on the nanoparticle, wherein the targeting ligand binds CD38, G-protein coupled receptor 18 (Gpr18), formyl peptide receptor 2 (Fpr2), CD64, or CD68.

10. The method of claim 7, wherein the administering treats an autoimmune disease in the subject.

11. The method of claim 1, wherein the immune cells are macrophages, regulatory T cells, myeloid-derived suppressor cells, regulatory dendritic cells, neutrophils, T helper 17 cells, regulatory B cells, mesenchymal stromal cells, or a combination thereof.

12. The method of claim 1, wherein the administering comprises locally administering.

13. The method of claim 1, wherein the administering comprises systemic administering.


SEQUENCE LISTING (111 sequences)

SEQ ID NO: 1 - protein, 498 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 2 - protein, 514 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 3 - protein, 504 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 4 - protein, 488 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 5 - protein, 412 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTVTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 6 - protein, 147 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYETPSPLRITLLVQERRRKKRKSCRGCCQA

SEQ ID NO: 7 - protein, 497 aa, Mus musculus
MNHSAPGIPPPPRRVRLKPWLVAQVNSCQYPGLQWVNGEKKLFYIPWRHATRHGPSQDGDNTIFKAWAKETGKYTEGVDEADPAKWKANLRCALNKSRDFQLFYDGPRDMPPQPYKIYEVCSNGPAPTESQPTDDYVLGEEEEEEEEELQRMLPGLSITEPALPGPPNAPYSLPKEDTKWPPALQPPVGLGPPVPDPNLLAPPSGNPAGFRQLLPEVLEPGPLASSQPPTEPLLPDLLISPHMLPLTDLEIKFQYRGRAPRTLTISNPQGCRLFYSQLEATQEQVELFGPVTLEQVRFPSPEDIPSDKQRFYTNQLLDVLDRGLILQLQGQDLYAIRLCQCKVFWSGPCALAHGSCPNPIQREVKTKLFSLEQFLNELILFQKGQTNTPPPFEIFFCFGEEWPDVKPREKKLITVQVVPVAARLLLEMFSGELSWSADSIRLQISNPDLKDHMVEQFKELHHLWQSQQQLQPMVQAPPVAGLDASQGPWPMHPVGMQ

SEQ ID NO: 8 - protein, 325 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 9 - protein, 427 aa, Homo sapiens
MGTPKPRILPWLVSQLDLGQLEGVAWVNKSRTRFRIPWKHGLRQDAQQEDFGIFQAWAEATGAYVPGRDKPDLPTWKRNFRSALNRKEGLRLAEDRSKDPHDPHKIYEFVNSGVGDFSQPDTSPDTNGGGSTSDTQEDILDELLGNMVLAPLPDPGPPSLAVAPEPCPQPLRSPSLDNPTPFPNLGPSENPLKRLLVPGEEWEFEVTAFYRGRQVFQQTISCPEGLRLVGSEVGDRTLPGWPVTLPDPGMSLTDRGVMSYVRHVLSCLGGGLALWRAGQWLWAQRLGHCHTYWAVSEELLPNSGHGPDGEVPKDKEGGVFDLGPFIVDLITFTEGSGRSPRYALWFCVGESWPQDQPWTKRLVMVKVVPTCLRALVEMARVGGASSLENTVDLHISNSHPLSLTSDQYKAYLQDLVEGMDFQGPGES

SEQ ID NO: 10 - protein, 503 aa, Homo sapiens
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 11 - protein, 426 aa, Homo sapiens
MCDRNGGRRLRQWLIEQIDSSMYPGLIWENEEKSMFRIPWKHAGKQDYNQEVDASIFKAWAVFKGKFKEGDKAEPATWKTRLRCALNKSPDFEEVTDRSQLDISEPYKVYRIVPEEEQKCKLGVATAGCVNEVTEMECGRSEIDELIKEPSVDDYMGMIKRSPSPPEACRSQLLPDWWAQQPSTGVPLVTGYTTYDAHHSAFSQMVISFYYGGKLVGQATTTCPEGCRLSLSQPGLPGTKLYGPEGLELVRFPPADAIPSERQRQVTRKLFGHLERGVLLHSSRQGVFVKRLCQGRVFCSGNAVVCKGRPNKLERDEVVQVFDTSQFFRELQQFYNSQGRLPDGRVVLCFGEEFPDMAPLRSKLILVQIEQLYVRQLAEEAGKSCGAGSVMQAPEEPPPDQVFRMFPDICASHQRSFFRENQQITV

SEQ ID NO: 12 - protein, 329 aa, Mus musculus
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTRNQRKERKSKSSRDTKSKTKRKLCGDVSPDTFSDGLSSSTLPDDHSSYTTQGYLGQDLDMERDITPALSPCVVSSSLSEWHMQMDIIPDSTTDLYNLQVSPMPSTSEAATDEDEEGKIAEDLMKLFEQSEWQPTHIDGKGYLLNEPGTQLSSVYGDFSCKEEPEIDSPRGDIGIGIQHVFTEMKNMDSIMWMDSLLGNSVRLPPSIQAIPCAP

SEQ ID NO: 13 - protein, 419 aa, Mus musculus
METPKPRILPWLVSQLDLGQLEGVAWLDESRTRFRIPWKHGLRQDAQMADFGIFQAWAEASGAYTPGKDKPDVSTWKRNFRSALNRKEVLRLAADNSKDPYDPHKVYEFVTPGARDFVHLGASPDTNGKSSLPHSQENLPKLFDGLILGPLKDEGSSDLAIVSDPSQQLPSPNVNNFLNPAPQENPLKQLLAEEQWEFEVTAFYRGRQVFQQTLFCPGGLRLVGSTADMTLPWQPVTLPDPEGFLTDKLVKEYVGQVLKGLGNGLALWQAGQCLWAQRLGHSHAFWALGEELLPDSGRGPDGEVHKDKDGAVFDLRPFVADLIAFMEGSGHSPRYTLWFCMGEMWPQDQPWVKRLVMVKVVPTCLKELLEMAREGGASSLKTVDLHISNSQPISLTSDQYKAYLQDLVEDMDFQATGNI

SEQ ID NO: 14 - protein, 457 aa, Mus musculus
MAEVRGVQRVLFGDWLLGEVSSGQYEGLQWLNEARTVFRVPWKHFGRRDLDEEDAQIFKAWAVARGRWPPSGVNLPPPEAEAAERRERRGWKTNFRCALHSTGRFILRQDNSGDPVDPHKVYELSRELGSTVGPATENREEVSLSNALPTQGVSPGSFLARENAGLQTPSPLLSSDAGDLLLQVLQYSHILESESGADPVPPQAPGQEQDRVYEEPYAAWQVEAVPSPRPQQPALTERSLGFLDVTIMYKGRTVLQAVVGHPRCVFLYSPMAPAVRTSEPQPVIFPSPAELPDQKQLHYTETLLQHVSPGLQLELRGPSLWALRMGKCKVYWEVGSPMGTTGPSTPPQLLERNRHTPIFDFSTFFRELEEFRARRRQGSPHYTIYLGFGQDLSAGRPKEKTLILVKLEPWVCKAYLEGVQREGVSSLDSSSLGLCLSSTNSLYEDIEHFLMDLGQWP

SEQ ID NO: 15 - protein, 525 aa, Mus musculus
MAEVRGVQRVLFGDWLLGEVSSGQYEGLQWLNEARTVFRVPWKHFGRRDLDEEDAQIFKAWAVARGRWPPSGVNLPPPEAEAAERRERRGWKTNFRCALHSTGRFILRQDNSGDPVDPHKVYELSRELGSTVGPATENREEVSLSNALPTQGVSPGSFLARENAGLQTPSPLLSSDAGDLLLQVLQYSHILESESGADPVPPQAPGQEQDRVYEEPYAAWQVEAVPSPRPQQPALTERSLGFLDVTKLFDGLILGPLKDEGSSDLAIVSDPSQQLPSPNVNNFLNPAPQENPLKQLLAEEQWEFEVTAFYRGRQVFQQTLFCPGGLRLVGSTADMTLPWQPVTLPDPEGFLTDKLVKEYVGQVLKGLGNGLALWQAGQCLWAQRLGHSHAFWALGEELLPDSGRGPDGEVHKDKDGAVFDLRPFVADLIAFMEGSGHSPRYTLWFCMGEMWPQDQPWVKRLVMVKVVPTCLKELLEMAREGGASSLKTVDLHIDNDQPIDLDDDQYKAYLQDLVEDMDFQATGNI

SEQ ID NO: 16 - protein, 424 aa, Mus musculus
MCDRNGGRRLRQWLIEQIDSSMYPGLIWENDEKTMFRIPWKHAGKQDYNQEVDASIFKAWAVFKGKFKEGDKAEPATWKTRLRCALNKSPDFEEVTDRSQLDISEPYKVYRIVPEEEQKCKLGVAPAGCMSEVPEMECGRSEIEELIKEPSVDEYMGMTKRSPSPPEACRSQILPDWWVQQPSAGLPLVTGYAAYDTHHSAFSQMVISFYYGGKLVGQATTTCLEGCRLSLSQPGLPKLYGPDGLEPVCFPTADTIPSERQRQVTRKLFGHLERGVLLHSNRKGVFVKRLCQGRVFCSGNAVVCKGRPNKLERDEVVQVFDTNQFIRELQQFYATQSRLPDSRVVLCFGEEFPDTVPLRSKLILVQVEQLYARQLVEEAGKSCGAGSLMPALEEPQPDQAFRMFPDICTSHQRPFFRENQQITV

SEQ ID NO: 17 - protein, 424 aa, Mus musculus
MCDRNGGRRLRQWLIEQIDSSMYPGLIWENDEKTMFRIPWKHAGKQDYNQEVDASIFKAWAVFKGKFKEGDKAEPATWKTRLRCALNKSPDFEEVTDRSQLDISEPYKVYRIVPEEEQKCKLGVAPAGCMSEVPEMECGRSEIEELIKEPSVDEYMGMTKRSPSPPEACRSQILPDWWVQQPSAGLPLVTGYAAYDTHHSAFSQMVISFYYGGKLVGQATTTCLEGCRLSLSQPGLPKLYGPDGLEPVCFPTADTIPSERQRQVTRKLFGHLERGVLLHSNRKGVFVKRLCQGRVFCSGNAVVCKGRPNRLERDEVVQVFDTNQFIRELQQFYATQSRLPDSRVVLCFGEEFPDTVPLRSKLILVQVEQLYARQLVEEAGKSCGAGSLMPALEEPQPDQAFRMFPDICTSHQRPFFRENQQITV

SEQ ID NO: 18 - protein, 756 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 19 - protein, 754 aa, Homo sapiens
MFSGGCHSPGFGRPSPAFPAPGSPPPAPRPCRQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 20 - protein, 256 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQCVRMWPGTVAHSCNPSTLGGRGRWIS

SEQ ID NO: 21 - protein, 697 aa, Homo sapiens
MSSDGTIRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 22 - protein, 738 aa, Mus musculus
MSWSPSLPTQTCGAWEMKERLGTGGFGNVIRWHNQATGEQIAIKQCRQELSPKNRDRWCLEIQIMRRLNHPNVVAARDVPEGMQNLAPNDLPLLAMEYCQGGDLRRYLNQFENCCGLREGAVLTLLSDIASALRYLHENRIIHRDLKPENIVLQQGEKRLIHKIIDLGYAKELDQGSLCTSFVGTLQYLAPELLEQQKYTVTVDYWSFGTLAFECITGFRPFLPNWQPVQWHSKVRQKSEVDIVVSEDLNGAVKFSSSLPFPNNLNSVLAERLEKWLQLMLMWHPRQRGTDPQYGPNGCFRALDDILNLKLVHVLNMVTGTVHTYPVTEDESLQSLKTRIQEDTGILETDQELLQEAGLVLLPDKPATQCISDSKTNEGLTLDMDLVFLFDNSKINYETQITPRPQPESVSCILQEPKRNLSFFQLRKVWGQVWHSIQTLKEDCNRLQQGQRAAMMSLLRNNSCLSKMKNAMASTAQQLKAKLDFFKTSIQIDLEKYKEQTEFGITSDKLLLAWREMEQAVEQCGRENDVKHLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDERTVVRLQEKRQKELWNLLKIACSKVRGPVSGSPDSMNVSRLSHPGQLMSQPSSACDSLPESDKKSEELVAEAHALCSRLESALQDTVKEQDRSFTVTA

SEQ ID NO: 23 - DNA, 1497 bp, Homo sapiens
  source 1..1497: mol_type = unassigned DNA
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 24 - DNA, 1545 bp, Homo sapiens
  source 1..1545: mol_type = unassigned DNA
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagatgcagtgcagtctggcccccacatgacaccctattctttactcaaagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 25 - DNA, 1514 bp, Homo sapiens
  source 1..1514: mol_type = unassigned DNA
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagatgcagtgcagtctggcccccacatgacaccctattctttactcaaagaggatgtcaagtggccgcccactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 26 - DNA, 1467 bp, Homo sapiens
  source 1..1467: mol_type = unassigned DNA
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggccgcccactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 27 - DNA, 1239 bp, Homo sapiens
  source 1..1239: mol_type = unassigned DNA
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagtgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 28 - DNA, 444 bp, Homo sapiens
  source 1..444: mol_type = unassigned DNA
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgagactcccagccccctgaggattactcttttggtgcaggagaggaggaggaagaagaggaagagctgcagaggatgttgccaagcctga

SEQ ID NO: 29 - DNA, 1494 bp, Mus musculus
  source 1..1494: mol_type = unassigned DNA
atgaatcatagtgcacccgggatccctcctcctccaagacgagtacgcctcaagccctggttggtagctcaagtcaactcatgccaataccctgggcttcagtgggtgaacggtgagaagaaattgttttatatcccatggcgacacgcaacaagacatggcccatcacaggatggagataacaccatatttaaggcatgggcaaaggaaacaggaaagtacactgagggcgttgatgaggccgatcctgcaaaatggaaagcaaatttgcgatgcgctctcaataaatcacgagatttccaactcttttacgacggcccaagggacatgccaccacaaccttataaaatctacgaggtatgttccaacggtccagccccaactgaatcccagcctactgacgactatgtccttggagaagaggaggaagaagaagaggaggaacttcagcggatgttgcctgggttgtccataactgagcctgccttgccaggaccccctaatgcaccatactcccttcccaaagaagatacaaaatggccccccgcattgcaaccccccgttggtttgggaccacctgtgcccgacccaaatctcttggccccaccaagcggtaacccagccggatttcgacaacttctgcccgaagtccttgagccaggtcccttggcctcttctcagccccctacagaacctctgctccccgatctcttgatatctccccacatgcttcccttgactgatttggagataaaatttcagtatcgcggccgagctcccagaacactgactatatcaaatccccaaggttgccgcctgttttacagtcagttggaggcaactcaggagcaagtagagctctttgggccagttactctggagcaggtgagattccctagtccagaggacataccaagcgataagcaaagattttacacaaatcaacttctggatgtacttgatcgaggtttgatccttcagttgcagggccaagatttgtatgccattcgactctgtcaatgcaaggtattttggagcggcccatgtgcccttgctcatggcagctgccctaatcccatccaaagagaagtaaagactaaacttttcagcctggaacaatttctcaacgaactcattctgtttcaaaaaggtcagaccaacacaccccctcctttcgagattttcttctgcttcggcgaagagtggcctgatgtgaagccccgcgaaaaaaagcttatcaccgttcaagtggtacccgtcgcagccaggctccttcttgaaatgtttagcggtgaactctcatggtccgctgacagtatccggctccaaatatcaaaccctgatcttaaagaccacatggtagaacagtttaaagaactccaccacctgtggcaatcccaacagcagctccagccaatggttcaagctcctccagtcgctgggctggacgcctcacaaggaccctggcccatgcaccccgtcgggatgcagtaa

SEQ ID NO: 30 - DNA, 978 bp, Homo sapiens
  source 1..978: mol_type = unassigned DNA
atgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtaaggaggagccagaaattgacagcccagggggggatattgggctgagtctacagcgtgtcttcacagatctgaagaacatggatgccacctggctggacagcctgctgaccccagtccggttgccctccatccaggccattccctgtgcaccgtag

SEQ ID NO: 31 - DNA, 1284 bp, Homo sapiens
  source 1..1284: mol_type = unassigned DNA
atgggaaccccaaagccacggatcctgccctggctggtgtcgcagctggacctggggcaactggagggcgtggcctgggtgaacaagagccgcacgcgcttccgcatcccttggaagcacggcctacggcaggatgcacagcaggaggatttcggaatcttccaggcctgggccgaggccactggtgcatatgttcccgggagggataagccagacctgccaacctggaagaggaatttccgctctgccctcaaccgcaaagaagggttgcgtttagcagaggaccggagcaaggaccctcacgacccacataaaatctacgagtttgtgaactcaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagagtgggagttcgaggtgacagccttctaccggggccgccaagtcttccagcagaccatctcctgcccggagggcctgcggctggtggggtccgaagtgggagacaggacgctgcctggatggccagtcacactgccagaccctggcatgtccctgacagacaggggagtgatgagctacgtgaggcatgtgctgagctgcctgggtgggggactggctctctggcgggccgggcagtggctctgggcccagcggctggggcactgccacacatactgggcagtgagcgaggagctgctccccaacagcgggcatgggcctgatggcgaggtccccaaggacaaggaaggaggcgtgtttgacctggggcccttcattgtagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctga

SEQ ID NO: 32 - DNA, 1512 bp, Homo sapiens
  source 1..1512: mol_type = unassigned DNA
atggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctag

SEQ ID NO: 33 - DNA, 1281 bp, Homo sapiens
  source 1..1281: mol_type = unassigned DNA
atgtgtgaccggaatggtggtcggcggcttcgacagtggctgatcgagcagattgacagtagcatgtatccaggactgatttgggagaatgaggagaagagcatgttccggatcccttggaaacacgctggcaagcaagattataatcaggaagtggatgcctccatttttaaggcctgggcagtttttaaagggaagtttaaagaaggggacaaagctgaaccagccacttggaagacgaggttacgctgtgctttgaataagagcccagattttgaggaagtgacggaccggtcccaactggacatttccgagccatacaaagtttaccgaattgttcctgaggaagagcaaaaatgcaaactaggcgtggcaactgctggctgcgtgaatgaagttacagagatggagtgcggtcgctctgaaatcgacgagctgatcaaggagccttctgtggacgattacatggggatgatcaaaaggagcccttccccgccggaggcctgtcggagtcagctccttccagactggtgggcgcagcagcccagcacaggcgtgccgctggtgacggggtacaccacctacgacgcgcaccattcagcattctcccagatggtgatcagcttctactatgggggcaagctggtgggccaggccaccaccacctgccccgagggctgccgcctgtccctgagccagcctgggctgcccggcaccaagctgtatgggcccgagggcctggagctggtgcgcttcccgccggccgacgccatccccagcgagcgacagaggcaggtgacgcggaagctgttcgggcacctggagcgcggggtgctgctgcacagcagccggcagggcgtgttcgtcaagcggctgtgccagggccgcgtgttctgcagcggcaacgccgtggtgtgcaaaggcaggcccaacaagctggagcgtgatgaggtggtccaggtcttcgacaccagccagttcttccgagagctgcagcagttctataacagccagggccggcttcctgacggcagggtggtgctgtgctttggggaagagtttccggatatggcccccttgcgctccaaactcattctcgtgcagattgagcagctgtatgtccggcaactggcagaagaggctgggaagagctgtggagccggctctgtgatgcaggcccccgaggagccgccgccagaccaggtcttccggatgtttccagatatttgtgcctcacaccagagatcatttttcagagaaaaccaacagatcaccgtctaa

SEQ ID NO: 34 - DNA, 990 bp, Mus musculus
  source 1..990: mol_type = unassigned DNA
atgccaatcactcgaatgcggatgagaccctggctagagatgcagattaattccaaccaaatcccagggctgatctggatcaataaagaagagatgatcttccagattccatggaagcacgctgctaagcacggctgggacatcaacaaggatgcctgtctgttccggagctgggccattcacacaggccgatacaaagcaggagaaaaagagccagatcccaagacatggaaggcaaacttccgttgtgccatgaactccctgccagacatcgaggaagtgaaggatcagagtaggaacaagggcagctctgctgtgcgggtgtaccggatgctgccacccctcaccaggaaccagaggaaagagagaaagtccaagtccagccgagacactaagagcaaaaccaagaggaagctgtgtggagatgttagcccggacactttctctgatggactcagcagctctaccctacctgatgaccacagcagttacaccactcagggctacctgggtcaggacttggatatggaaagggacataactccagcactgtcaccgtgtgtcgtcagcagcagtctctctgagtggcatatgcagatggacattataccagatagcaccactgatctgtataacctacaggtgtcacccatgccttccacctccgaagccgcaacagacgaggatgaggaagggaagatagccgaagaccttatgaagctctttgaacagtctgagtggcagccgacacacatcgatggcaagggatacttgctcaatgagccagggacccagctctcttctgtctatggagacttcagctgcaaagaggaaccagagattgacagccctcgaggggacattgggataggcatacaacatgtcttcacggagatgaagaatatggactccatcatgtggatggacagcctgctgggcaactctgtgaggctgccgccctctattcaggccattccttgtgcaccatag

SEQ ID NO: 35 - DNA, 1260 bp, Mus musculus
  source 1..1260: mol_type = unassigned DNA
atggaaaccccgaaaccgcggattttgccctggctggtgtcacagctggacctggggcagctggaaggcgtggcctggctggacgagagccgaacgaggttcaggatcccgtggaagcatggcctacggcaggacgcacagatggctgactttggcatcttccaggcctgggcagaagccagtggtgcctacaccccggggaaggataagccggacgtgtcaacctggaagaggaatttccggtcagccctgaaccggaaagaagtgttgcggttagctgctgacaatagcaaggacccttatgaccctcataaagtgtatgagtttgtgactccaggggcgcgggacttcgtacatctgggtgcctctcctgacaccaatggcaaaagcagcctgcctcactcccaggaaaacctaccgaagttatttgatggcctgatcttggggcccctcaaagatgaggggtcctcagatctggctattgtttctgatccttctcaacaactgccaagccccaatgtgaacaacttcctaaaccctgcaccccaagaaaatccactgaagcagctgctagctgaggaacaatgggagttcgaggtgaccgccttctaccgaggccgccaggtcttccagcagacactcttttgcccggggggcctgcggctggtgggcagcacagctgacatgacactgccctggcagccagtcaccctgcccgatcctgaggggtttctgacggacaagcttgtgaaggagtacgtggggcaggtgctcaaagggctgggcaatgggctggcactgtggcaggctgggcagtgcctctgggcccagcgcctaggccactcccacgccttctgggctctgggggaggagctgcttccagacagtgggcgagggcctgatggagaggtccacaaggacaaggacggagccgtgttcgacctcaggcccttcgtggcagatctgattgccttcatggaaggaagtggacactccccacgctacactctgtggttctgcatgggggaaatgtggccccaggaccagccatgggtcaagaggcttgtgatggtcaaggttgttcctacatgtcttaaggagctgttagagatggcccgggaagggggagcctcttcactgaaaaccgtggacttgcacatctccaacagccagcctatctcccttacctctgaccagtacaaggcctacctccaggacttggtggaggacatggacttccaggccactggaaatatctga

SEQ ID NO: 36 - DNA, 1374 bp, Mus musculus
  source 1..1374: mol_type = unassigned DNA
atggctgaagtgaggggggtccagcgagtgctgtttggagactggctattgggggaggtcagcagcggccagtacgaggggctgcagtggctgaacgaggctcgcacagtcttccgcgtaccctggaagcatttcggtcgtagggatctggatgaagaagatgcacagatcttcaaggcctgggctgtggcccgagggaggtggccacctagtggagttaacctgccacccccagaggctgaggctgctgagcgaagagagcgaagaggctggaagaccaacttccgctgtgcactccacagcacagggcgttttatcttgcgccaagacaattcaggggatccagttgatccgcataaggtgtacgaacttagccgggagcttggatctactgtgggcccagccacggaaaatagggaagaagtgagcctcagcaatgctctgcccacacagggtgtgtccccaggatcatttctggcaagagaaaatgctgggctccaaaccccaagccctctgctttctagtgatgccggggacctcttgcttcaggttctgcagtacagccacatactggaatccgagtctggggcagaccccgtcccaccacaggctcctggccaggagcaagaccgtgtttacgaggaaccctatgcagcatggcaggtggaagctgtccccagtcccaggcctcaacagccagctctcaccgagcgcagccttgggttcctggatgtgaccatcatgtacaagggccgcacagtgctacaggcagtggtggggcaccccagatgcgtgttcctgtacagccccatggccccagcagtaagaacttcagagccccagccggtgatctttcccagtcctgctgagctcccagatcagaagcagctgcactacacagagacgcttctccagcatgtgtctcccggccttcagctggagcttcgaggaccgtcactgtgggccctgcgtatgggcaagtgcaaggtgtactgggaggtaggcagccctatgggcactaccggcccctccaccccaccccagctgctggagcgcaaccgccacacccccatcttcgacttcagcactttcttccgagaactggaggagtttcgggctcggaggcggcaagggtcaccacactacaccatctacctgggttttgggcaagacttgtcagcagggaggcccaaggagaagaccctgatcctggtgaagctggagccatgggtatgcaaggcatacctggagggcgtgcagcgtgagggtgtgtcctccctggacagcagcagtctcggcttgtgcttgtctagcaccaacagtctctacgaagacatcgaacacttcctcatggacctgggtcagtggccttga

SEQ ID NO: 37 - DNA, 1578 bp, Mus musculus
  source 1..1578: mol_type = unassigned DNA
atggccgaagttcgaggagtacagcgcgtgctgtttggggactggttgcttggtgaagtctcttctggtcagtatgaaggcctgcaatggcttaatgaggcacgcacagtttttcgagtgccatggaaacacttcggtaggcgcgatctcgacgaagaggatgcccagattttcaaggcatgggcagtcgcacggggcaggtggcccccttcaggcgtaaatttgccccccccagaggctgaagctgctgaacgcagagaacgccggggatggaagactaactttcgatgtgcccttcacagtacaggcaggttcatcttgcggcaggataatagtggcgaccctgtagacccacacaaggtttatgagctgagccgggagcttggctcaacagtcggtcctgcaaccgagaacagagaagaggtgtccttgtctaacgccctcccaactcagggtgtgtctcccggtagcttcctggcacgcgaaaacgctggactccaaaccccctccccactgttgtccagtgatgccggtgatcttctccttcaggtgctccaatactcccatatactggagagcgagtcaggggctgatcccgtgccccctcaagctcctggacaggaacaagatcgcgtctacgaggagccatatgctgcctggcaggtcgaggctgtgccatcacctcggcctcaacagcccgctctcaccgagcgctcacttgggtttttggatgtcactaaacttttcgacggcctgatacttggcccattgaaggacgagggatcatccgatcttgccatagtaagtgacccatcacagcagttgccctcaccaaacgtcaacaacttcctcaatccagctccccaggagaaccccctcaaacagcttctcgcagaagagcaatgggagtttgaggtgactgctttctatagaggtaggcaggtgttccaacaaactctgttttgccccggaggtctgcgccttgtaggtagcaccgcagacatgacacttccctggcaacctgtgacacttcccgatcctgagggatttctcacagataaactcgttaaggaatatgtggggcaagtactcaaaggtctgggcaatgggttggccctttggcaagctggtcaatgtctctgggctcaacgactcgggcactcacatgctttttgggctcttggcgaggagctgctccccgacagcgggcgcggacctgacggggaggttcataaggacaaagacggcgccgtatttgatcttagacccttcgtggcagatctgatcgctttcatggaaggatcaggtcatagccccaggtacacactttggttttgcatgggtgaaatgtggcctcaggaccaaccttgggtcaagcgcttggtcatggttaaggtggttcccacttgcctcaaagagttgttggagatggctagggaaggtggggcttcctcactgaaaaccgtagatctccacattgataatgatcagcctatagatttggacgacgaccaatacaaagcttatctccaggacctggttgaagatatggactttcaggctacaggtaacatctaa

SEQ ID NO: 38 - DNA, 1275 bp, Mus musculus
  source 1..1275: mol_type = unassigned DNA
atgtgtgacaggaacggcggtagaagactgagacagtggctgatcgagcaaattgacagctcaatgtaccctgggttgatatgggaaaacgatgaaaagacaatgttcagaataccctggaagcacgctggaaagcaggattacaaccaggaagtggacgccagtatttttaaggcttgggctgtcttcaaagggaagtttaaagagggcgacaaagcagagccagcaacctggaaaacccgcttgaggtgtgcactcaataagtcacccgacttcgaggaagtcactgaccgcagtcaattggacatatcagaaccatacaaagtctacaggatagtccccgaagaagagcagaaatgcaaactcggtgtagcacctgctggctgtatgagtgaagtgcctgaaatggaatgcggcagatcagaaatcgaagaactcataaaagaaccaagtgtagatgagtatatgggaatgaccaaaagatccccatcccccccagaagcctgtcggagccaaatcttgcctgactggtgggtacagcaaccctccgccggacttccccttgtgacaggctatgccgcttacgatactcatcacagcgcttttagccagatggttatttccttctattatggaggaaaactggtcggccaagccacaaccacctgcctcgaggggtgtcgcttgagtttgagtcaacccggtcttcccaaactctatggccccgatgggcttgaacctgtctgctttcccactgctgatactattccctcagagagacaacgacaagtcacccgaaaattgtttggccacctcgagaggggagtactcttgcactctaacaggaagggtgtctttgtgaaacgcctctgtcaaggtagggtattctgttctggaaatgcagttgtttgcaaaggcaggcctaacaaactggaacgggatgaagtcgtacaagtgttcgataccaatcagtttattcgggagttgcagcagttttacgctacacaaagtcgcctccctgacagtcgggttgtgttgtgcttcggggaggagtttcccgacactgtacccctccgaagcaaactcatactggtacaggtagaacaactttatgccaggcaactggtggaagaggccggtaagtcctgtggcgcaggatccctgatgccagccctggaagagccccagcctgaccaagcatttaggatgtttcccgacatttgtacctcacaccagaggccttttttccgcgaaaaccagcagataaccgtgtaa

SEQ ID NO: 39 - DNA, 1275 bp, Mus musculus
  source 1..1275: mol_type = unassigned DNA
atgtgtgacaggaacggcggtagaagactgagacagtggctgatcgagcaaattgacagctcaatgtaccctgggttgatatgggaaaacgatgaaaagacaatgttcagaataccctggaagcacgctggaaagcaggattacaaccaggaagtggacgccagtatttttaaggcttgggctgtcttcaaagggaagtttaaagagggcgacaaagcagagccagcaacctggaaaacccgcttgaggtgtgcactcaataagtcacccgacttcgaggaagtcactgaccgcagtcaattggacatatcagaaccatacaaagtctacaggatagtccccgaagaagagcagaaatgcaaactcggtgtagcacctgctggctgtatgagtgaagtgcctgaaatggaatgcggcagatcagaaatcgaagaactcataaaagaaccaagtgtagatgagtatatgggaatgaccaaaagatccccatcccccccagaagcctgtcggagccaaatcttgcctgactggtgggtacagcaaccctccgccggacttccccttgtgacaggctatgccgcttacgatactcatcacagcgcttttagccagatggttatttccttctattatggaggaaaactggtcggccaagccacaaccacctgcctcgaggggtgtcgcttgagtttgagtcaacccggtcttcccaaactctatggccccgatgggcttgaacctgtctgctttcccactgctgatactattccctcagagagacaacgacaagtcacccgaaaattgtttggccacctcgagaggggagtactcttgcactctaacaggaagggtgtctttgtgaaacgcctctgtcaaggtagggtattctgttctggaaatgcagttgtttgcaaaggcaggcctaacagactggaacgggatgaagtcgtacaagtgttcgataccaatcagtttattcgggagttgcagcagttttacgctacacaaagtcgcctccctgacagtcgggttgtgttgtgcttcggggaggagtttcccgacactgtacccctccgaagcaaactcatactggtacaggtagaacaactttatgccaggcaactggtggaagaggccggtaagtcctgtggcgcaggatccctgatgccagccctggaagagccccagcctgaccaagcatttaggatgtttcccgacatttgtacctcacaccagaggccttttttccgcgaaaaccagcagataaccgtgtaa

SEQ ID NO: 40 - DNA, 2271 bp, Homo sapiens
  source 1..2271: mol_type = unassigned DNA
atgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 41 - DNA, 2265 bp, Homo sapiens
  source 1..2265: mol_type = unassigned DNA
atgttttcaggggggtgtcatagccccgggtttggccgccccagccccgccttccccgccccggggagcccgccccctgccccgcgtccctgccgacaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 42 - DNA, 771 bp, Homo sapiens
  source 1..771: mol_type = unassigned DNA
atgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtgcgtaagaatgtggccgggtacagtggctcactcctgtaatcccagcactttgggaggccgaggcaggtggatcagttga

SEQ ID NO: 43 - DNA, 2094 bp, Homo sapiens
  source 1..2094: mol_type = unassigned DNA
atgtcatccgatggcacaatcaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 44 - DNA, 2220 bp, Mus musculus
  source 1..2220: mol_type = unassigned DNA
atgagctggtcaccgtccctcccaacccagacatgtggagcctgggaaatgaaagaacgcctggggaccgggggatttggaaacgtcatccggtggcacaatcaggcgacaggtgaacagatcgccatcaagcaatgccgacaggagctcagcccaaagaacagagaccgctggtgcctcgaaatccagatcatgagaaggctgaaccatcccaatgtggtggctgcccgggatgtcccagaggggatgcagaacctggcacccaatgatttgccactgctggccatggagtactgccaaggaggagatctccgaagatacttgaaccagttcgagaactgctgtggcctgcgggaaggagctgtccttaccctgctgagtgacatcgcatcggctcttagataccttcacgaaaacagaatcatccatcgagacctgaagccagaaaacatcgttctgcagcaaggagagaaaagattaatacacaaaattattgatctaggatatgccaaggagctggatcagggcagtctgtgcacgtcatttgtggggactctgcaatacctggcgccagagcttctggagcagcagaagtacaccgtgaccgttgactactggagcttcggcaccctggccttcgagtgcatcactggcttccggcccttcctccctaactggcagcctgtgcagtggcactccaaagtccggcagaagagcgaagtggacatcgttgttagtgaagacttgaatggagcagtgaagttttcaagttcgctacccttccccaataatcttaacagtgtcttggctgaacggctggagaagtggctgcagctgatgcttatgtggcaccctcggcaaaggggcacggatccccagtatggccccaacggctgcttcagagccctggatgacatcttgaacttgaagctggttcatgtcttgaacatggtcacaggcaccgttcacacataccccgtgacggaggatgagagtctgcagagcttaaaaaccagaatccaggaagacacggggatcctggagacagaccaggagctgctgcaagaggcagggctggtgctgctccctgacaagcctgctactcagtgcatctcagacagcaagacaaacgagggcctcacgttggacatggatcttgtttttctctttgacaacagtaaaatcaactatgagactcagatcaccccccgaccccaaccggaaagtgtcagctgtatccttcaggagcccaagcggaacctctccttcttccagctgaggaaagtgtggggccaagtctggcacagcatccagacgctgaaggaagactgtaaccggctgcagcagggacagcgagcagccatgatgagtctcctccggaataacagctgcctctctaagatgaagaacgccatggcctccacggcccagcagctcaaggccaagctggacttcttcaaaaccagcatccagatcgacctggagaagtataaagagcagaccgagtttgggatcacctcagataaattgctgctggcttggcgggagatggagcaggctgtggagcagtgtgggcgggagaatgacgtgaagcatctagtagagcggatgatggcactgcagactgacattgtggacctgcagaggagcccgatgggtcggaagcaggggggcaccctggatgacctagaggaacaagcgagggagctctaccgaagactcagggagaagccaagagaccaaaggacagaaggtgacagccaggagatggtacggctgctgcttcaggcaatccaaagctttgagaagaaagttcgggtgatttatacacagctcagtaagaccgtggtttgtaagcagaaggcactggagttgctgcccaaggtagaagaggtagtgagccttatgaacgaggacgagaggaccgtggtccggcttcaggagaagcggcagaaggaactctggaacctcctgaagatcgcctgtagcaaagtccgaggtcccgtgagtggaagcccagacagcatgaatgtgtctcgactcagtcaccctggtcagctaatgtcccagccttccagtgcctgtgacagcttacctgaatcagacaagaaaagtgaagaactggtggccgaagcccacgccctctgctcccggctagaaagtgcgctgcaggacactgtgaaggagcaagacagaagcttcacggtaaccgcctgataa

SEQ ID NO: 45 - protein, 11 aa, synthetic construct
  REGION 1..11: note = CDRH1 that binds CD206
SGNIFSINAIG

SEQ ID NO: 46 - protein, 9 aa, synthetic construct
  REGION 1..9: note = CDRH2 that binds CD206
TITLSGSTN

SEQ ID NO: 47 - protein, 11 aa, synthetic construct
  REGION 1..11: note = CDRH3 that binds CD206
NTYSDSDVYGY

SEQ ID NO: 48 - protein, 11 aa, synthetic construct
  REGION 1..11: note = CDRH1 that binds CD206
PGFKLDYYAIA

SEQ ID NO: 49 - protein, 9 aa, synthetic construct
  REGION 1..9: note = CDRH2 that binds CD206
SINSSGGST

SEQ ID NO: 50 - protein, 16 aa, synthetic construct
  REGION 1..16: note = CDRH3 that binds CD206
LRRYYGLNLDPGSYDY

SEQ ID NO: 51 - protein, 10 aa, synthetic construct
  REGION 1..10: note = CDRH1 that binds CD206
GFPFNIYPMS

SEQ ID NO: 52 - protein, 9 aa, synthetic construct
  REGION 1..9: note = CDRH2 that binds CD206
YISHGGTTT

SEQ ID NO: 53 - protein, 12 aa, synthetic construct
  REGION 1..12: note = CDRH3 that binds CD206
GYARLMTDSELV

SEQ ID NO: 54 - protein, 9 aa, synthetic construct
  REGION 1..9: note = CDRL1 that binds CD163
ASQSVSHDV

SEQ ID NO: 55 - skipped

SEQ ID NO: 56 - protein, 8 aa, synthetic construct
  REGION 1..8: note = CDRL3 that binds CD163
QDYSSPRT

SEQ ID NO: 57 - protein, 8 aa, synthetic construct
  REGION 1..8: note = CDRH1 that binds CD163
GYSITSDY

SEQ ID NO: 58 - skipped

SEQ ID NO: 59 - protein, 12 aa, synthetic construct
  REGION 1..12: note = CDRH3 that binds CD163
CVSGTYYFDYWG

SEQ ID NO: 60 - protein, 9 aa, synthetic construct
  REGION 1..9: note = CDRL1 that binds CD163
ASQSVSSDV

SEQ ID NO: 61 - skipped

SEQ ID NO: 62 - protein, 8 aa, synthetic construct
  REGION 1..8: note = CDRL3 that binds CD163
QDYTSPRT

SEQ ID NO: 63 - protein, 8 aa, synthetic construct
  REGION 1..8: note = CDRH1 that binds CD163
GYSITSDY

SEQ ID NO: 64 - skipped

SEQ ID NO: 65 - protein, 12 aa, synthetic construct
  REGION 1..12: note = CDRH3 that binds CD163
CVSGTYYFDYWG

SEQ ID NO: 66 - protein, 16 aa, synthetic construct
  REGION 1..16: note = CDRL1 that bind CD23
RSSKSLLYKDGKTYLN

SEQ ID NO: 67 - protein, 7 aa, synthetic construct
  REGION 1..7: note = CDRL2 that bind CD23
LMSTRAS

SEQ ID NO: 68 - protein, 9 aa, synthetic construct
  REGION 1..9: note = CDRL3 that bind CD23
QQLVEYPFT

SEQ ID NO: 69 - protein, 5 aa, synthetic construct
  REGION 1..5: note = CDRH1 that bind CD23
GYWMS

SEQ ID NO: 70 - protein, 19 aa, synthetic construct
  REGION 1..19: note = CDRH2 that bind CD23
EIRLKSDNYATHYAESVKG

SEQ ID NO: 71 - skipped

SEQ ID NO: 72 - protein, 8 aa, synthetic construct
  REGION 1..8: note = CDRL1 that bind CD38
SSNIGDNY

SEQ ID NO: 73 - skipped

SEQ ID NO: 74 - protein, 10 aa, synthetic construct
  REGION 1..10: note = CDRL3 that bind CD38
QSYDSSLSGS

SEQ ID NO: 75 - protein, 8 aa, synthetic construct
  REGION 1..8: note = CDRH1 that bind CD38
GFTFDDYG

SEQ ID NO: 76 - protein, 8 aa, synthetic construct
  REGION 1..8: note = CDRH2 that binds CD38
ISWNGGKT

SEQ ID NO: 77 - protein, 16 aa, synthetic construct
  REGION 1..16: note = CDRH3 that binds CD38
ARGSLFHDSSGFYFGH

SEQ ID NO: 78 - protein, 8 aa, synthetic construct
  REGION 1..8: note = CDRL1 that binds CD38
NSNIGSNT

SEQ ID NO: 79 - skipped

SEQ ID NO: 80 - protein, 11 aa, synthetic construct
  REGION 1..11: note = CDRL3 that binds CD38
QSYDSSLSGSR

SEQ ID NO: 81 - protein, 8 aa, synthetic construct
  REGION 1..8: note = CDRH1 that binds CD38
GFTFNNYG

SEQ ID NO: 82 - protein, 8 aa, synthetic construct
  REGION 1..8: note = CDRH2 that binds CD38
ISYDGSDK

SEQ ID NO: 83 - protein, 15 aa, synthetic construct
  REGION 1..15: note = CDRH3 that binds CD38
ARVYYYGFSGPSMDV

SEQ ID NO: 84 - protein, 11 aa, synthetic construct
  REGION 1..11: note = CDRL1 that binds CD38
RASQSVSSYLA

SEQ ID NO: 85 - protein, 7 aa, synthetic construct
  REGION 1..7: note = CDRL2 that binds CD38
DASNRAT

SEQ ID NO: 86 - protein, 10 aa, synthetic construct
  REGION 1..10: note = CDRL3 that binds CD38
QQRSNWPPTF

SEQ ID NO: 87 - protein, 5 aa, synthetic construct
  REGION 1..5: note = CDRH1 that binds CD38
SFAMS

SEQ ID NO: 88 - protein, 17 aa, synthetic construct
  REGION 1..17: note = CDRH2 that binds CD38
AISGSGGGTYYADSVKG

SEQ ID NO: 89 - protein, 13 aa, synthetic construct
  REGION 1..13: note = CDRH3 that binds CD38
DKILWFGEPVFDY

SEQ ID NO: 90 - protein, 11 aa, synthetic construct
  REGION 1..11: note = CDRL1 that binds CD64
RASQSVSSYLA

SEQ ID NO: 91 - protein, 7 aa, synthetic construct
  REGION 1..7: note = CDRL2 that binds CD64
DASSRAT

SEQ ID NO: 92 - protein, 10 aa, synthetic construct
  REGION 1..10: note = CDRL3 that binds CD64
QLRSNWPPYT

SEQ ID NO: 93 - protein, 5 aa, synthetic construct
  REGION 1..5: note = CDRH1 that binds CD64
GYGMH

SEQ ID NO: 94 - protein, 17 aa, synthetic construct
  REGION 1..17: note = CDRH2 that binds CD64
VIWYDGSNKYYADSVKG

SEQ ID NO: 95 - protein, 9 aa, synthetic construct
  REGION 1..9: note = CDRH3 that binds CD64
DTGDRFFDY

SEQ ID NO: 96 - DNA, 20 bp, synthetic construct
  misc_feature 1..20: note = Fw primer SerpinB2
  source 1..20: mol_type = other DNA
actggggcagttatgacagg

SEQ ID NO: 97 - DNA, 19 bp, synthetic construct
  misc_feature 1..19: note = Rv primer SerpinB2
  source 1..19: mol_type = other DNA
gatgatcggccacaaactg

SEQ ID NO: 98 - DNA, 21 bp, synthetic construct
  misc_feature 1..21: note = Fw primer Retnla
  source 1..21: mol_type = other DNA
ttgttcccttctcatctgcat

SEQ ID NO: 99 - DNA, 21 bp, synthetic construct
  misc_feature 1..21: note = Rv primer Retnla
  source 1..21: mol_type = other DNA
ccttgaccttattctccacga

SEQ ID NO: 100 - DNA, 19 bp, synthetic construct
  misc_feature 1..19: note = Fw primer Ccl5
  source 1..19: mol_type = other DNA
cctactcccactcggtcct

SEQ ID NO: 101 - DNA, 21 bp, synthetic construct
  misc_feature 1..21: note = Rv primer Ccl5
  source 1..21: mol_type = other DNA
ctgatttcttgggtttgctgt

SEQ ID NO: 102 - DNA, 18 bp, synthetic construct
  misc_feature 1..18: note = Fw primer Ccl11
  source 1..18: mol_type = other DNA
agagctccacagcgcttc

SEQ ID NO: 103 - DNA, 18 bp, synthetic construct
  misc_feature 1..18: note = Rv primer Cdl11
  source 1..18: mol_type = other DNA
cagcacctgggaggtgaa

SEQ ID NO: 104 - DNA, 26 bp, synthetic construct
  misc_feature 1..26: note = Fw primer Codon optimized IRF5
  source 1..26: mol_type = other DNA
tcttaaagaccacatggtagaacagt

SEQ ID NO: 105 - DNA, 18 bp, synthetic construct
  misc_feature 1..18: note = Rv primer codon optimized IRF5
  source 1..18: mol_type = other DNA
agctgctgttgggattgc

SEQ ID NO: 106 - DNA, 20 bp, synthetic construct
  misc_feature 1..20: note = Fw primer endogenous IRF5
  source 1..20: mol_type = other DNA
gctgtgcccttaacaaaagc

SEQ ID NO: 107 - DNA, 18 bp, synthetic construct
  misc_feature 1..18: note = RV primer endogenous IRF5
  source 1..18: mol_type = other DNA
ggctgaggtggcatgtct

SEQ ID NO: 108 - DNA, 19 bp, synthetic construct
  misc_feature 1..19: note = Fw primer GAPD
  source 1..19: mol_type = other DNA
agccacatcgctcagacac

SEQ ID NO: 109 - DNA, 19 bp, synthetic construct
  misc_feature 1..19: note = Rv primer GAPD
  source 1..19: mol_type = other DNA
gcccaatacgaccaaatcc

SEQ ID NO: 110 - protein, 134 aa, Homo sapiens
MNTEMYQTPMEVAVYQLHNFSISFFSSLLGGDVVSVKLDNSASGASVVAIDNKIEQAMDLVKNHLMYAVREEVEILKEQIRELVEKNSQLERENTLLKTLASPEQLEKFQSCLSPEEPAPESPQVPEAPGGSAV

SEQ ID NO: 111 - DNA, 405 bp, Homo sapiens
  source 1..405: mol_type = unassigned DNA
atgaacaccgaaatgtatcagacccccatggaggtggcggtctaccagctgcacaatttctccatctccttcttctcttctctgcttggaggggatgtggtttccgttaagctggacaacagtgcctccggagccagcgtggtggccatagacaacaagatcgaacaggccatggatctggtgaagaatcatctgatgtatgctgtgagagaggaggtggagatcctgaaggagcagatccgagagctggtggagaagaactcccagctagagcgtgagaacaccctgttgaagaccctggcaagcccagagcagctggagaagttccagtcctgtctgagccctgaagagccagctcccgaatccccacaagtgcccgaggcccctggtggttctgcggtgtaa